SEQUENCE LISTING (22 sequences)

SEQ ID NO: 1 - DNA, 1796 bp, Homo sapiens
  CDS (4)..(1509)
gcgatgtcgctcgtgctgctaagcctggccgcgctgtgcaggagcgccgtaccccgagagccgaccgttcaatgtggctctgaaactgggccatctccagagtggatgctacaacatgatctaatcccgggagacttgagggacctccgagtagaacctgttacaactagtgttgcaacaggggactattcaattttgatgaatgtaagctgggtactccgggcagatgccagcatccgcttgttgaaggccaccaagatttgtgtgacgggcaaaagcaacttccagtcctacagctgtgtgaggtgcaattacacagaggccttccagactcagaccagaccctctggtggtaaatggacattttcctatatcggcttccctgtagagctgaacacagtctatttcattggggcccataatattcctaatgcaaatatgaatgaagatggcccttccatgtctgtgaatttcacctcaccaggctgcctagaccacataatgaaatataaaaaaaagtgtgtcaaggccggaagcctgtgggatccgaacatcactgcttgtaagaagaatgaggagacagtagaagtgaacttcacaaccactcccctgggaaacagatacatggctcttatccaacacagcactatcatcgggttttctcaggtgtttgagccacaccagaagaaacaaacgcgagcttcagtggtgattccagtgactggggatagtgaaggtgctacggtgcagctgactccatattttcctacttgtggcagcgactgcatccgacataaaggaacagttgtgctctgcccacaaacaggcgtccctttccctctggataacaacaaaagcaagccgggaggctggctgcctctcctcctgctgtctctgctggtggccacatgggtgctggtggcagggatctatctaatgtggaggcacgaaaggatcaagaagacttccttttctaccaccacactactgccccccattaaggttcttgtggtttacccatctgaaatatgtttccatcacacaatttgttacttcactgaatttcttcaaaaccattgcagaagtgaggtcatccttgaaaagtggcagaaaaagaaaatagcagagatgggtccagtgcagtggcttgccactcaaaagaaggcagcagacaaagtcgtcttccttctttccaatgacgtcaacagtgtgtgcgatggtacctgtggcaagagcgagggcagtcccagtgagaactctcaagacctcttcccccttgcctttaaccttttctgcagtgatctaagaagccagattcatctgcacaaatacgtggtggtctactttagagagattgatacaaaagacgattacaatgctctcagtgtctgccccaagtaccacctcatgaaggatgccactgctttctgtgcagaacttctccatgtcaagcagcaggtgtcagcaggaaaaagatcacaagcctgccacgatggctgctgctccttgtagcccacccatgagaagcaagagaccttaaaggcttcctatcccaccaattacagggaaaaaacgtgtgatgatcctgaagcttactatgcagcctacaaacagccttagtaattaaaacattttataccaataaaattttcaaatattgctaactaatgtagcattaactaacgattggaaactacatttacaacttcaaagctgttttatacatagaaatcaattacagctttaattgaaaactgtaaccattttgataatgcaacaataaagcatcttcagcc

SEQ ID NO: 2 - protein, 502 aa, Homo sapiens
MSLVLLSLAALCRSAVPREPTVQCGSETGPSPEWMLQHDLIPGDLRDLRVEPVTTSVATGDYSILMNVSWVLRADASIRLLKATKICVTGKSNFQSYSCVRCNYTEAFQTQTRPSGGKWTFSYIGFPVELNTVYFIGAHNIPNANMNEDGPSMSVNFTSPGCLDHIMKYKKKCVKAGSLWDPNITACKKNEETVEVNFTTTPLGNRYMALIQHSTIIGFSQVFEPHQKKQTRASVVIPVTGDSEGATVQLTPYFPTCGSDCIRHKGTVVLCPQTGVPFPLDNNKSKPGGWLPLLLLSLLVATWVLVAGIYLMWRHERIKKTSFSTTTLLPPIKVLVVYPSEICFHHTICYFTEFLQNHCRSEVILEKWQKKKIAEMGPVQWLATQKKAADKVVFLLSNDVNSVCDGTCGKSEGSPSENSQDLFPLAFNLFCSDLRSQIHLHKYVVVYFREIDTKDDYNALSVCPKYHLMKDATAFCAELLHVKQQVSAGKRSQACHDGCCSL

SEQ ID NO: 3 - DNA, 637 bp, Mus musculus
  CDS (1)..(210)
gatttcagcagccagacgcatctgcacaaatacctggaggtctatcttgggggagcagacctcaaaggcgactataatgccctgagtgtctgcccccaatatcatctcatgaaggacgccacagctttccacacagaacttctcaaggctacgcagagcatgtcagtgaagaaacgctcacaagcctgccatgatagctgttcacccttgtagtccacccgggggaatagagactctgaagccttcctactctcccttccagtgacaaatgctgtgtgacgactctgaaatgtgtgggagaggctgtgtggaggtagtgctatgtacaaacttgctttaaaactggagtttgcaaagtcaacctgagcatacacgcctgaggctagtcattggctggatttatgaagacaacacagttacagacaataatgagtgggacctacatttgggatatacccaaagctgggtaatgattatcactgagaaccacgcactctggccatgaggtaatacggcacttccctgtcaggctgtctgtcaggttgggtctgtcttgcactgcccatgctctatgctgcacgtagaccgttttgtaacattttaatctgttaatgaataatccgtttgggaggctctc

SEQ ID NO: 4 - protein, 70 aa, Mus musculus
DFSSQTHLHKYLEVYLGGADLKGDYNALSVCPQYHLMKDATAFHTELLKATQSMSVKKRSQACHDSCSPL

SEQ ID NO: 5 - DNA, 2308 bp, Homo sapiens
  CDS (181)..(2289)
gagtcaggactcccaggacagagagtgcacaaactacccagcacagccccctccgccccctctggaggctgaagagggattccagcccctgccacccacagacacgggctgactggggtgtctgccccccttgggggcanccacagggcctcaggcctgggtgccacctggcactagaagatgcctgtgccctggttcttgctgtccttggcactgggccgaagccagtggatcctttctctggagaggcttgtggggcctcaggacgctacccactgctctccgggcctctcctgccgcctctgggacagtgacatactctgcctgcctggggacatcgtgcctgctccgggccccgtgctggcgcctacgcacctgcagacagagctggtgctgaggtgccagaaggagaccgactgtgacctctgtctgcgtgtggctgtccacttggccgtgcatgggcactgggaagagcctgaagatgaggaaaagtttggaggagcagctgacttaggggtggaggagcctaggaatgcctctctccaggcccaagtcgtgctctccttccaggcctaccctactgcccgctgcgtcctgctggaggtgcaagtgcctgctgcccttgtgcagtttggtcagtctgtgggctctgtggtatatgactgcttcgaggctgccctagggagtgaggtacgaatctggtcctatactcagcccaggtacgagaaggaactcaaccacacacagcagctgcctgactgcagggggctcgaagtctggaacagcatcccgagctgctgggccctgccctggctcaacgtgtcagcagatggtgacaacgtgcatctggttctgaatgtctctgaggagcagcacttcggcctctccctgtactggaatcaggtccagggccccccaaaaccccggtggcacaaaaacctgactggaccgcagatcattaccttgaaccacacagacctggttccctgcctctgtattcaggtgtggcctctggaacctgactccgttaggacgaacatctgccccttcagggaggacccccgcgcacaccagaacctctggcaagccgcccgactgcgactgctgaccctgcagagctggctgctggacgcaccgtgctcgctgcccgcagaagcggcactgtgctggcgggctccgggtggggacccctgccagccactggtcccaccgctttcctgggagaatgtcactgtggacgtgaacagctcggagaagctgcagctgcaggagtgcttgtgggctgactccctggggcctctcaaagacgatgtgctactgttggagacacgaggcccccaggacaacagatccctctgtgccttggaacccagtggctgtacttcactacccagcaaagcctccacgagggcagctcgccttggagagtacttactacaagacctgcagtcaggccagtgtctgcagctatgggacgatgacttgggagcgctatgggcctgccccatggacaaatacatccacaagcgctgggccctcgtgtggctggcctgcctactctttgccgctgcgctttccctcatcctccttctcaaaaaggatcacgcgaaagggtggctgaggctcttgaaacaggacgtccgctcgggggcggccgccaggggccgcgcggctctgctcctctactcagccgatgactcgggtttcgagcgcctggtgggcgccctggcgtcggccctgtgccagctgccgctgcgcgtggccgtagacctgtggagccgtcgtgaactgagcgcgcaggggcccgtggcttggtttcacgcgcagcggcgccagaccctgcaggagggcggcgtggtggtcttgctcttctctcccggtgcggtggcgctgtgcagcgagtggctacaggatggggtgtccgggcccggggcgcacggcccgcacgacgccttccgcgcctcgctcagctgcgtgctgcccgacttcttgcagggccgggcgcccggcagctacgtgggggcctgcttcgacaggctgctccacccggacgccgtacccgcccttttccgcaccgtgcccgtcttcacactgccctcccaactgccagacttcctgggggccctgcagcagcctcgcgccccgcgttccgggcggctccaagagagagcggagcaagtgtcccgggcccttcagccagccctggatagctacttccatcccccggggacntccgcgccgggacgcggggtgggaccaggggcgggacctggggcgggggacgggacttaaataaaggcagacgctg

SEQ ID NO: 6 - protein, 703 aa, Homo sapiens
  misc_feature (664)..(664): The ′Xaa′ at location 664 stands for Thr.
MPVPWFLLSLALGRSQWILSLERLVGPQDATHCSPGLSCRLWDSDILCLPGDIVPAPGPVLAPTHLQTELVLRCQKETDCDLCLRVAVHLAVHGHWEEPEDEEKFGGAADLGVEEPRNASLQAQVVLSFQAYPTARCVLLEVQVPAALVQFGQSVGSVVYDCFEAALGSEVRIWSYTQPRYEKELNHTQQLPDCRGLEVWNSIPSCWALPWLNVSADGDNVHLVLNVSEEQHFGLSLYWNQVQGPPKPRWHKNLTGPQIITLNHTDLVPCLCIQVWPLEPDSVRTNICPFREDPRAHQNLWQAARLRLLTLQSWLLDAPCSLPAEAALCWRAPGGDPCQPLVPPLSWENVTVDVNSSEKLQLQECLWADSLGPLKDDVLLLETRGPQDNRSLCALEPSGCTSLPSKASTRAARLGEYLLQDLQSGQCLQLWDDDLGALWACPMDKYIHKRWALVWLACLLFAAALSLILLLKKDHAKGWLRLLKQDVRSGAAARGRAALLLYSADDSGFERLVGALASALCQLPLRVAVDLWSRRELSAQGPVAWFHAQRRQTLQEGGVVVLLFSPGAVALCSEWLQDGVSGPGAHGPHDAFRASLSCVLPDFLQGRAPGSYVGACFDRLLHPDAVPALFRTVPVFTLPSQLPDFLGALQQPRAPRSGRLQERAEQVSRALQPALDSYFHPPGXSAPGRGVGPGAGPGAGDGT

SEQ ID NO: 7 - DNA, 2314 bp, Mus musculus
  CDS (199)..(2292)
ccaaatcgaaagcacgggagctgatactgggcctggagtccaggctcactggagtggggaagcatggctggagaggaattctagcccttgctctctcccagggacacggggctgattgtcagcaggggcgaggggtctgcccccccttgggggggcaggacggggcctcaggcctgggtgctgtccggcacctggaagatgcctgtgtcctggttcctgctgtccttggcactgggccgaaaccctgtggtcgtctctctggagagactgatggagcctcaggacactgcacgctgctctctaggcctctcctgccacctctgggatggtgacgtgctctgcctgcctggaagcctccagtctgccccaggccctgtgctagtgcctacccgcctgcagacggagctggtgctgaggtgtccacagaagacagattgcgccctctgtgtccgtgtggtggtccacttggccgtgcatgggcactgggcagagcctgaagaagctggaaagtctgattcagaactccaggagtctaggaacgcctctctccaggcccaggtggtgctctccttccaggcctaccccatcgcccgctgtgccctgctggaggtccaggtgcccgctgacctggtgcagcctggtcagtccgtgggttctgcggtatttgactgtttcgaggctagtcttggggctgaggtacagatctggtcctacacgaagcccaggtaccagaaagagctcaacctcacacagcagctgcctgactgcaggggtcttgaagtccgggacagcatccagagctgctgggtcctgccctggctcaatgtgtctacagatggtgacaatgtccttctgacactggatgtctctgaggagcaggactttagcttcttactgtacctgcgtccagtcccggatgctctcaaatccttgtggtacaaaaacctgactggacctcagaacattactttaaaccacacagacctggttccctgcctctgcattcaggtgtggtcgctagagccagactctgagagggtcgaattctgccccttccgggaagatcccggtgcacacaggaacctctggcacatagccaggctgcgggtactgtccccaggggtatggcagctagatgcgccttgctgtctgccgggcaaggtaacactgtgctggcaggcaccagaccagagtccctgccagccacttgtgccaccagtgccccagaagaacgccactgtgaatgagccacaagatttccagttggtggcaggccaccccaacctctgtgtccaggtgagcacctgggagaaggttcagctgcaagcgtgcttgtgggctgactccttggggcccttcaaggatgatatgctgttagtggagatgaaaaccggcctcaacaacacatcagtctgtgccttggaacccagtggctgtacaccactgcccagcatggcctccacgagagctgctcgcctgggagaggagttgctgcaagacttccgatcacaccagtgtatgcagctgtggaacgatgacaacatgggatcgctatgggcctgccccatggacaagtacatccacaggcgctgggtcctagtatggctggcctgcctactcttggctgcggcgcttttcttcttcctccttctaaaaaaggaccgcaggaaagcggcccgtggctcccgcacggccttgctcctccactccgccgacggagcgggctacgagcgcctggtgggagcactggcgtccgcgttgagccagatgccactgcgcgtggccgtggacctgtggagccgccgcgagctgagcgcgcacggagccctagcctggttccaccaccagcgacgccgtatcctgcaggagggtggcgtggtaatccttctcttctcgcccgcggccgtggcgcagtgtcagcagtggctgcagctccagacagtggagcccgggccgcatgacgccctcgccgcctggctcagctgcgtgctacccgatttcctgcaaggccgggcgaccggccgctacgtcggggtctacttcgacgggctgctgcacccagactctgtgccctccccgttccgcgtcgccccgctcttctccctgccctcgcagctgccggctttcctggatgcactgcagggaggctgctccacttccgcggggcgacccgcggaccgggtggaacgagtgacccaggcgctgcggtccgccctggacagctgtacttctagctcggaagccccaggctgctgcgaggaatgggacctgggaccctgcactacactagaataaaagccgatacagtattcct

SEQ ID NO: 8 - protein, 698 aa, Mus musculus
MPVSWFLLSLALGRNPVVVSLERLMEPQDTARCSLGLSCHLWDGDVLCLPGSLQSAPGPVLVPTRLQTELVLRCPQKTDCALCVRVVVHLAVHGHWAEPEEAGKSDSELQESRNASLQAQVVLSFQAYPIARCALLEVQVPADLVQPGQSVGSAVFDCFEASLGAEVQIWSYTKPRYQKELNLTQQLPDCRGLEVRDSIQSCWVLPWLNVSTDGDNVLLTLDVSEEQDFSFLLYLRPVPDALKSLWYKNLTGPQNITLNHTDLVPCLCIQVWSLEPDSERVEFCPFREDPGAHRNLWHIARLRVLSPGVWQLDAPCCLPGKVTLCWQAPDQSPCQPLVPPVPQKNATVNEPQDFQLVAGHPNLCVQVSTWEKVQLQACLWADSLGPFKDDMLLVEMKTGLNNTSVCALEPSGCTPLPSMASTRAARLGEELLQDFRSHQCMQLWNDDNMGSLWACPMDKYIHRRWVLVWLACLLLAAALFFFLLLKKDRRKAARGSRTALLLHSADGAGYERLVGALASALSQMPLRVAVDLWSRRELSAHGALAWFHHQRRRILQEGGVVILLFSPAAVAQCQQWLQLQTVEPGPHDALAAWLSCVLPDFLQGRATGRYVGVYFDGLLHPDSVPSPFRVAPLFSLPSQLPAFLDALQGGCSTSAGRPADRVERVTQALRSALDSCTSSSEAPGCCEEWDLGPCTTLE

SEQ ID NO: 9 - DNA, 2786 bp, Homo sapiens
  CDS (70)..(2283)
cccacgcntccgggccagcagcgggcggccggggcgcagagaacggcctggctgggcgagcgcacggccatggccccgtggctgcagctctgctccgtcttctttacggtcaacgcctgcctcaacggctcgcagctggctgtngccgctggcgggtccggccgcgcgcngggcgccgacacctgtagctggangggagtggggccagccagcagaaacagtgggctgtacaacatcaccttcaaatatgacaattgtaccacctacttgaatccagtggggaagcatgtgattgctgacgcccagaatatcaccatcagccagtatgcttgccatgaccaagtggcagtcaccattctttggtccccaggggccctcggcatcgaattcctgaaaggatttcgggtaatactggaggagctgaagtcggagggaagacagngccaacaactgattctaaaggatccgaagcagntcaacagtagcttcaaaagaactggaatggaatctcaacctttnctgaatatgaaatttgaaacggattatttcgtaaggttgtccttttccttcattaaaaacgaaagcaattaccaccctttcttctttagaacccgagcctgtgacctgttgttacagccggacaatctagcttgtaaacccttctggaagcctcggaacctgaacatcagccagcatggctcggacatgcaggtgtccttcgaccacgcaccgcacaacttcggcttccgtttcttctatcttcactacaagctcaagcacgaaggacctttcaagcgaaagacctgtaagcaggagcaaactacagagatgaccagctgcctccttcaaaatgtttctccaggggattatataattgagctggtggatgacactaacacaacaagaaaagtgatgcattatgccttaaagccagtgcactccccgtgggccgggcccatcagagccgtggccatcacagtgccactggtagtcatatcggcattcgcgacgctcttcactgtgatgtgccgcaagaagcaacaagaaaatatatattcacatttagatgaagagagctctgagtcttccacatacactgcagcactcccaagagagaggctccggccgcggccgaaggtctttctctgctattccagtaaagatggccagaatcacatgaatgtcgtccagtgtttcgcctacttcctccaggacttctgtggctgtgaggtggctctggacctgtgggaagacttcagcctctgtagagaagggcagagagaatgggtcatccagaagatccacgagtcccagttcatcattgtggtttgttccaaaggtatgaagtactttgtggacaagaagaactacaaacacaaaggaggtggccgaggctcggggaaaggagagctcttcctggtggcggtgtcagccattgccgaaaagctccgccaggccaagcagagttcgtccgcggcgctcagcaagtttatcgccgtctactttgattattcctgcgagggagacgtccccggtatcctagacctgagtaccaagtacagactcatggacaatcttcctcagctctgttcccacctgcactcccgagaccacggcctccaggagccggggcagcacacgcgacagggcagcagaaggaactacttccggagcaagtcaggccggtccctatacgtcgccatttgcaacatgcaccagtttattgacgaggagcccgactggttcgaaaagcagttcgttcccttccatcctcctccactgcgctaccgggagccagtcttggagaaatttgattcgggcttggttttaaatgatgtcatgtgcaaaccagggcctgagagtgacttctgcctaaaggtagaggcggctgttcttggggcaaccggaccagccgactcccagcacgagagtcagcatgggggcctggaccaagacggggaggcccggcctgcccttgacggtagcgccgccctgcaacccctgctgcacacggtgaaagccggcagcccctcggacatgccgcgggactcaggcatctatgactcgtctgtgccctcatccgagctgtctctgccactgatggaaggactctcgacggaccagacagaaacgtcttccctgacggagagcgtgtcctcctcttcaggcctgggtgaggaggaacctcctgcccttccttccaagctcctctcttctgggtcatgcaaagcagatcttggttgccgcagctacactgatgaactccacgcggtcgcccctttgtaacaaaacgaaagagtctaagcattgccactttagctgctgcctccctctgattccccagctcatctccctggttgcatggcccacttggagctgaggtctcatacaaggatatttggagtgaaatgctggccagtacttgttctcccttgccccaaccctttaccggatatcttgacaaactctccaattttctaaaatgatatggagctctgaaaggcatgtccataaggtctgacaacagcttgccaaatttggttagtccttggatcagagcctgttgtgggaggtagggaggaaatatgtaaagaaaaacaggaagatacctgcactaatcattcagacttcattgagctctgcaaactttgcctgtttgctattggctaccttgatttgaaatgctttgtgaaaaaaggcacttttaacatcatagccacagaaatcaagtgccagtctatctggaatccatgttgtattgcagataatgttctcatttatttttg

SEQ ID NO: 10 - protein, 738 aa, Homo sapiens
  misc_feature (18)..(18): The ′Xaa′ at location 18 stands for Gln, Arg, Pro, or Leu.
MAPWLQLCSVFFTVNACLNGSQLAVAAGGSGRAXGADTCSWXGVGPASRNSGLYNITFKYDNCTTYLNPVGKHVIADAQNITISQYACHDQVAVTILWSPGALGIEFLKGFRVILEELKSEGRQXQQLILKDPKQXNSSFKRTGMESQPXLNMKFETDYFVRLSFSFIKNESNYHPFFFRTRACDLLLQPDNLACKPFWKPRNLNISQHGSDMQVSFDHAPHNFGFRFFYLHYKLKHEGPFKRKTCKQEQTTEMTSCLLQNVSPGDYIIELVDDTNTTRKVMHYALKPVHSPWAGPIRAVAITVPLVVISAFATLFTVMCRKKQQENIYSHLDEESSESSTYTAALPRERLRPRPKVFLCYSSKDGQNHMNVVQCFAYFLQDFCGCEVALDLWEDFSLCREGQREWVIQKIHESQFIIVVCSKGMKYFVDKKNYKHKGGGRGSGKGELFLVAVSAIAEKLRQAKQSSSAALSKFIAVYFDYSCEGDVPGILDLSTKYRLMDNLPQLCSHLHSRDHGLQEPGQHTRQGSRRNYFRSKSGRSLYVAICNMHQFIDEEPDWFEKQFVPFHPPPLRYREPVLEKFDSGLVLNDVMCKPGPESDFCLKVEAAVLGATGPADSQHESQHGGLDQDGEARPALDGSAALQPLLHTVKAGSPSDMPRDSGIYDSSVPSSELSLPLMEGLSTDQTETSSLTESVSSSSGLGEEEPPALPSKLLSSGSCKADLGCRSYTDELHAVAPL

SEQ ID NO: 11 - DNA, 2012 bp, Homo sapiens
  CDS (1)..(1971)
atggggagctccagactggcagccctgctcctgcctctcctcctcatagtcatcgacctctctgactctgctgggattggctttcgccacctgccccactggaacacccgctgtcctctggcctcccacacggaagttctgcctatatcccttgccgcacctggtgggccctcttctccacaaagccttggtgtgtgcgagtctggcactgttcccgctgtttgtgccagcatctgctgtcaggtggctcaggtcttcaacggggcctcttccacctcctggtgcagaaatccaaaaagtcttccacattcaagttctataggagacacaagatgccagcacctgctcagaggaagctgctgcctcgtcgtcacctgtctgagaagagccatcacatttccatcccctccccagacatctcccacaagggacttcgctctaaaaggacccaaccttcggatccagagacatgggaaagtcttcccagattggactcacaaaggcatggaggtgggcactgggtacaacaggagatgggttcagctgagtggtggacccgagttctcctttgatttgctgcctgaggcccgggctattcgggtgaccatatcttcaggccctgaggtcagcgtgcgtctttgtcaccagtgggcactggagtgtgaagagctgagcagtccctatgatgtccagaaaattgtgtctgggggccacactgtagagctgccttatgaattccttctgccctgtctgtgcatagaggcatcctacctgcaagaggacactgtgaggcgcaaaaaatgtcccttccagagctggccagaagcctatggctcggacttctggaagtcagtgcacttcactgactacagccagcacactcagatggtcatggccctgacactccgctgcccactgaagctggaagctgccctctgccagaggcacgactggcataccctttgcaaagacctcccgaatgccacggctcgagagtcagatgggtggtatgttttggagaaggtggacctgcacccccagctctgcttcaaggtacaaccatggttctcttttggaaacagcagccatgttgaatgcccccaccagactgggtctctcacatcctggaatgtaagcatggatacccaagcccagcagctgattcttcacttctcctcaagaatgcatgccaccttcagtgctgcctggagcctcccaggcttggggcaggacactttggtgccccccgtgtacactgtcagccaggtgtggcggtcagatgtccagtttgcctggaagcacctcttgtgtccagatgtctcttacagacacctggggctcttgatcctggcactgctggccctcctcaccctactgggtgttgttctggccctcacctgccggcgcccacagtcaggcccgggcccagcgcggccagtgctcctcctgcacgcggcggactcggaggcgcagcggcgcctggtgggagcgctggctgaactgctacgggcagcgctgggcggcgggcgcgacgtgatcgtggacctgtgggaggggaggcacgtggcgcgcgtgggcccgctgccgtggctctgggcggcgcggacgcgcgtagcgcgggagcagggcactgtgctgctgctgtggagcggcgccgaccttcgcccggtcagcggccccgacccccgcgccgcgcccctgctcgccctgctccacgctgccccgcgcccgctgctgctgctcgcttacttcagtcgcctctgcgccaagggcgacatccccccgccgctgcgcgccctgccgcgctaccgcctgctgcgcgacctgccgcgtctgctgcgggcgctggacgcgcggcctttcgcagaggccaccagctggggccgccttggggcgcggcagcgcaggcagagccgcctagagctgtgcagccggctcgaacgagaggccgcccgacttgcagacctaggttgagcagagctccaccgcagtcccgggtgtctgcggccgct

SEQ ID NO: 12 - protein, 657 aa, Homo sapiens
MGSSRLAALLLPLLLIVIDLSDSAGIGFRHLPHWNTRCPLASHTEVLPISLAAPGGPSSPQSLGVCESGTVPAVCASICCQVAQVFNGASSTSWCRNPKSLPHSSSIGDTRCQHLLRGSCCLVVTCLRRAITFPSPPQTSPTRDFALKGPNLRIQRHGKVFPDWTHKGMEVGTGYNRRWVQLSGGPEFSFDLLPEARAIRVTISSGPEVSVRLCHQWALECEELSSPYDVQKIVSGGHTVELPYEFLLPCLCIEASYLQEDTVRRKKCPFQSWPEAYGSDFWKSVHFTDYSQHTQMVMALTLRCPLKLEAALCQRHDWHTLCKDLPNATARESDGWYVLEKVDLHPQLCFKVQPWFSFGNSSHVECPHQTGSLTSWNVSMDTQAQQLILHFSSRMHATFSAAWSLPGLGQDTLVPPVYTVSQVWRSDVQFAWKHLLCPDVSYRHLGLLILALLALLTLLGVVLALTCRRPQSGPGPARPVLLLHAADSEAQRRLVGALAELLRAALGGGRDVIVDLWEGRHVARVGPLPWLWAARTRVAREQGTVLLLWSGADLRPVSGPDPRAAPLLALLHAAPRPLLLLAYFSRLCAKGDIPPPLRALPRYRLLRDLPRLLRALDARPFAEATSWGRLGARQRRQSRLELCSRLEREAARLADLG

SEQ ID NO: 13 - DNA, 808 bp, Mus musculus
  CDS (78)..(806)
cagctccgggccaggccctgctgccctcttgcagacaggaaagacatggtctctgcgccctgatcctacagaagctcatggggagccccagactggcagccttgctcctgtctctcccgctactgctcatcggcctcgctgtgtctgctcgggttgcctgcccctgcctgcggagttggaccagccactgtctcctggcctaccgtgtggataaacgttttgctggccttcagtggggctggttccctctcttggtgaggaaatctaaaagtcctcctaaatttgaagactattggaggcacaggacaccagcatccttccagaggaagctgctaggcagcccttccctgtctgaggaaagccatcgaatttccatcccctcctcagccatctcccacagaggccaacgcaccaaaagggcccagccttcagctgcagaaggaagagaacatctccctgaagcagggtcacaaaagtgtggaggacctgaattctcctttgatttgctgcccgaggtgcaggctgttcgggtgactattcctgcaggccccaaggcacgtgtgcgcctttgttatcagtgggcactggaatgtgaagacttgagtagcccttttgatacccagaaaattgtgtctggagggcacactgtagacctgccttatgaattccttctgccctgcatgtgcatagaggcctcctacctgcaagaggacactgtgaggcgcaaaagtgtcccttccagagctggcctgaagcttatggctcagacttctggcagtcaatacgcttcactgactacagccagcac

SEQ ID NO: 14 - protein, 243 aa, Mus musculus
MGSPRLAALLLSLPLLLIGLAVSARVACPCLRSWTSHCLLAYRVDKRFAGLQWGWFPLLVRKSKSPPKFEDYWRHRTPASFQRKLLGSPSLSEESHRISIPSSAISHRGQRTKRAQPSAAEGREHLPEAGSQKCGGPEFSFDLLPEVQAVRVTIPAGPKARVRLCYQWALECEDLSSPFDTQKIVSGGHTVDLPYEFLLPCMCIEASYLQEDTVRRKSVPSRAGLKLMAQTSGSQYASLTTAS

SEQ ID NO: 15 - DNA, 2377 bp, Homo sapiens
  CDS (180)..(1874)
ttttgagcagaggcttcctaggctccgtagaaatttgcatacagcttccacttcctgcttcagagcctgttcttctacttacctgggcccggagaaggtggagggagacgagaagccgccgagagccgactaccctccgggcccagtctgtctgtccgtggtggatctaagaaactagaatgaaccgaagcattcctgtggaggttgatgaatcagaaccatacccaagtcagttgctgaaaccaatcccagaatattccccggaagaggaatcagaaccacctgctccaaatataaggaacatggcacccaacagcttgtctgcacccacaatgcttcacaattcctccggagacttttctcaagctcactcaaccctgaaacttgcaaatcaccagcggcctgtatcccggcaggtcacctgcctgcgcactcaagttctggaggacagtgaagacagtttctgcaggagacacccaggcctgggcaaagctttcccttctgggtgctctgcagtcagcgagcctgcgtctgagtctgtggttggagccctccctgcagagcatcagttttcatttatggaaaaacgtaatcaatggctggtatctcagctttcagcggcttctcctgacactggccatgactcagacaaatcagaccaaagtttacctaatgcctcagcagactccttgggcggtagccaggagatggtgcaacggccccagcctcacaggaaccgagcaggcctggatctgccaaccatagacacgggatatgattcccagccccaggatgtcctgggcatcaggcagctggaaaggcccctgcccctcacctccgtgtgttacccccaggacctccccagacctctcaggtccagggagttccctcagtttgaacctcagaggtatccagcatgtgcacagatgctgcctcccaatctttccccacatgctccatggaactatcattaccattgtcctggaagtcccgatcaccaggtgccatatggccatgactaccctcgagcagcctaccagcaagtgatccagccggctctgcctgggcagcccctgcctggagccagtgtgagaggcctgcaccctgtgcagaaggttatcctgaattatcccagcccctgggaccaagaagagaggcccgcacagagagactgctcctttccggggcttccaaggcaccaggaccagccacatcaccagccacctaatagagctggtgctcctggggagtccttggagtgccctgcagagctgagaccacaggttccccagcctccgtccccagctgctgtgcctagaccccctagcaaccctccagccagaggaactctaaaaacaagcaatttgccagaagaattgcggaaagtctttatcacttattcgatggacacagctatggaggtggtgaaattcgtgaactttttgttggtaaatggcttccaaactgcaattgacatatttgaggatagaatccgaggcattgatatcattaaatggatggagcgctaccttagggataagaccgtgatgataatcgtagcaatcagccccaaatacaaacaggacgtggaaggcgctgagtcgcagctggacgaggatgagcatggcttacatactaagtacattcatcgaatgatgcagattgagttcataaaacaaggaagcatgaatttcagattcatccctgtgctcttcccaaatgctaagaaggagcatgtgcccacctggcttcagaacactcatgtctacagctggcccaagaataaaaaaaacatcctgctgcggctgctgagagaggaagagtatgtggctcctccacgggggcctctgcccacccttcaggtggttcccttgtgacaccgttcatccccagatcactgaggccaggccatgtttggggccttgttctgacagcattctggctgaggctggtcggtagcactcctggctggtttttttctgttcctccccgagaggccctctggcccccaggaaacctgttgtgcagagctcttccccggagacctccacacaccctggctttgaagtggagtctgtgactgctctgcattctctgcttttaaaaaaaccattgcaggtgccagtgtcccatatgttcctcctgacagtttgatgtgtccattctgggcctctcagtgcttagcaagtagataatgtaagggatgtggcagcaaatggaaatgactacaaacactctcctatcaatcacttcaggctacttttatgagttagccagatgcttgtgtatcctcagaccaaactgattcatgtacaaataataaaatgtttactcttttgtaaaaaaaaaaaaaaaaaaaaaaagaaaaaaaaaaaaa

SEQ ID NO: 16 - protein, 565 aa, Homo sapiens
MNRSIPVEVDESEPYPSQLLKPIPEYSPEEESEPPAPNIRNMAPNSLSAPTMLHNSSGDFSQAHSTLKLANHQRPVSRQVTCLRTQVLEDSEDSFCRRHPGLGKAFPSGCSAVSEPASESVVGALPAEHQFSFMEKRNQWLVSQLSAASPDTGHDSDKSDQSLPNASADSLGGSQEMVQRPQPHRNRAGLDLPTIDTGYDSQPQDVLGIRQLERPLPLTSVCYPQDLPRPLRSREFPQFEPQRYPACAQMLPPNLSPHAPWNYHYHCPGSPDHQVPYGHDYPRAAYQQVIQPALPGQPLPGASVRGLHPVQKVILNYPSPWDQEERPAQRDCSFPGLPRHQDQPHHQPPNRAGAPGESLECPAELRPQVPQPPSPAAVPRPPSNPPARGTLKTSNLPEELRKVFITYSMDTAMEVVKFVNFLLVNGFQTAIDIFEDRIRGIDIIKWMERYLRDKTVMIIVAISPKYKQDVEGAESQLDEDEHGLHTKYIHRMMQIEFIKQGSMNFRFIPVLFPNAKKEHVPTWLQNTHVYSWPKNKKNILLRLLREEEYVAPPRGPLPTLQVVPL

SEQ ID NO: 17 - DNA, 1323 bp, Mus musculus
  CDS (1)..(1026)
caggacctccctgggcctctgaggtccagggaattgccacctcagtttgaacttgagaggtatccaatgaacgcccagctgctgccgccccatccttccccacaggccccatggaactgtcagtactactgccccggagggccctaccaccaccaggtgccacacggccatggctaccctccagcagcagcctaccagcaagtactccagcctgctctgcctgggcaggtccttcctggggcaagggcaagaggcccacgccctgtgcagaaggtcatcctgaatgactccagcccccaagaccaagaagagagacctgcacagagagacttctctttcccgaggctcccgagggaccagctctaccgcccaccatctaatggagtggaagcccctgaggagtccttggaccttcctgcagagctgagaccacatggtccccaggctccatccctagctgccgtgcctagaccccctagcaaccccttagcccgaggaactctaagaaccagcaatttgccagaagaattacggaaagtctttatcacttattctatggacacagccatggaggtggtgaaatttgtgaactttctgttggtgaacggcttccaaactgcgattgacatatttgaggatagaatccggggtattgatatcattaaatggatggagcgctatcttcgagataagacagtgatgataatcgtagcaatcagccccaaatacaaacaggatgtggaaggcgctgagtcgcagctggacgaggacgagcatggcttacatactaagtacattcatcggatgatgcagattgagttcataagtcagggaagcatgaacttcagattcatccctgtgctcttcccaaatgccaagaaggagcatgtgccgacctggcttcagaacactcatgtttacagctggcccaagaataagaaaaacatcctgctgcggctgctcagggaggaagagtatgtggctcctccccgaggccctctgcccacccttcaggtggtacccttgtgacgatggccactccagctcagtgccagcctgttctcacagcattcttctagcggagctggctggtggcacccaggccctggaacacctcttctacagagtcctctgtctcctgagtctgagttgtcctcgctgggcttccagagcttcagtgcctggatgctgcaggtgacagaaacaaacatctatgaccacaaaaactctcatcacttcagctacttttatgagtcggtcagatgctctgtgtccttagaccagtctaaatcatgctcaaataataaaatgattattctttgt

SEQ ID NO: 18 - protein, 342 aa, Mus musculus
QDLPGPLRSRELPPQFELERYPMNAQLLPPHPSPQAPWNCQYYCPGGPYHHQVPHGHGYPPAAAYQQVLQPALPGQVLPGARARGPRPVQKVILNDSSPQDQEERPAQRDFSFPRLPRDQLYRPPSNGVEAPEESLDLPAELRPHGPQAPSLAAVPRPPSNPLARGTLRTSNLPEELRKVFITYSMDTAMEVVKFVNFLLVNGFQTAIDIFEDRIRGIDIIKWMERYLRDKTVMIIVAISPKYKQDVEGAESQLDEDEHGLHTKYIHRMMQIEFISQGSMNFRFIPVLFPNAKKEHVPTWLQNTHVYSWPKNKKNILLRLLREEEYVAPPRGPLPTLQVVPL

SEQ ID NO: 19 - protein, 207 aa, Homo sapiens
RKVWIIYSADHPLYVDVVLKFAQFLLTACGTEVALDLLEEQAISEAGVMTWVGRQKQEMVESNSKIIVLCSRGTRAKWQALLGRGAPVRLRCDHGKPVGDLFTAAMNMILPDFKRPACFGTYVVCYFSEVSCDGDVPDLFGAAPRYPLMDRFEEVYFRIQDLEMFQPGRMHRVGELSGDNYLRSPGGRQLRAALDRFRDWQVRCPDW

SEQ ID NO: 20 - protein, 208 aa, Mus musculus
RKVWIVYSADHPLYVEVVLKFAQFLITACGTEVALDLLEEQVISEVGVMTWVSRQKQEMVESNSKIIILCSRGTQAKWKAILGWAEPAVQLRCDHWKPAGDLFTAAMNMILPDFKRPACFGTYVVCYFSGICSERDVPDLFNITSRYPLMDRFEEVYFRIQDLEMFEPGRMHHVRELTGDNYLQSPSGRQLKEAVLRFQEWQTQCPDW

SEQ ID NO: 21 - protein, 190 aa, Caenorhabditis elegans
VKVMIVYADDNDLHTDCVKKLVENLRNCASCDPVFDLEKLITAEIVPSRWLVDQISSLKKFIIVVSDCAEKILDTEASETHQLVQARPFADLFGPAMEMIIRDATHNFPEARKKYAVVRFNYSPHVPPNLAILNLPTFIPEQFAQLTAFLHNVEHTERANVTQNISEAQIHEWNLCASRMMSFFVRNPNW

SEQ ID NO: 22 - protein, 178 aa, Caenorhabditis elegans
FKVMLVCPEVSGRDEDFMMRIADALKKSNNKVVCDRWFEDSKNAEENMLHWVYEQTKIAEKIIVFHSAYYHPRCGIYDVINNFFPCTDPRLAHIALTPEAQRSVPKEVEYVLPRDQKLLEDAFDITIADPLVIDIPIEDVAIPENVPIHHESCDSIDSRNNSKTHSTDSGVSSLSSNS

(19) United States
(12) Patent Application Publication
(10) Pub. No.: US 2003/0092881 A1
(43) Pub. Date: May 15, 2003

(54) Mammalian receptor proteins; related reagents and methods

(76) Inventor: Daniel M. Gorman, Newark, CA (US)

Correspondence Address: DNAX RESEARCH INSTITUTE, LEGAL DEPARTMENT, 901 CALIFORNIA AVENUE, PALO ALTO, CA, 94304, US

(21) Appl. No.: 09/863,818

(22) Filed: May 23, 2001

Related U.S. Application Data
(60) Provisional application No. 60/206,862, filed on May 24, 2000.

(51) Int. Cl. C07K 14/705; C07H 21/04; C12P 21/02; C12N 5/06

(52) U.S. Cl. 530/350; 435/69.1; 435/325; 435/320.1; 536/23.5

(57) ABSTRACT

Nucleic acids encoding mammalian, e.g., primate, receptors, purified receptor proteins and fragments thereof. Antibodies, both polyclonal and monoclonal, are also provided. Methods of using the compositions for both diagnostic and therapeutic utilities are described.

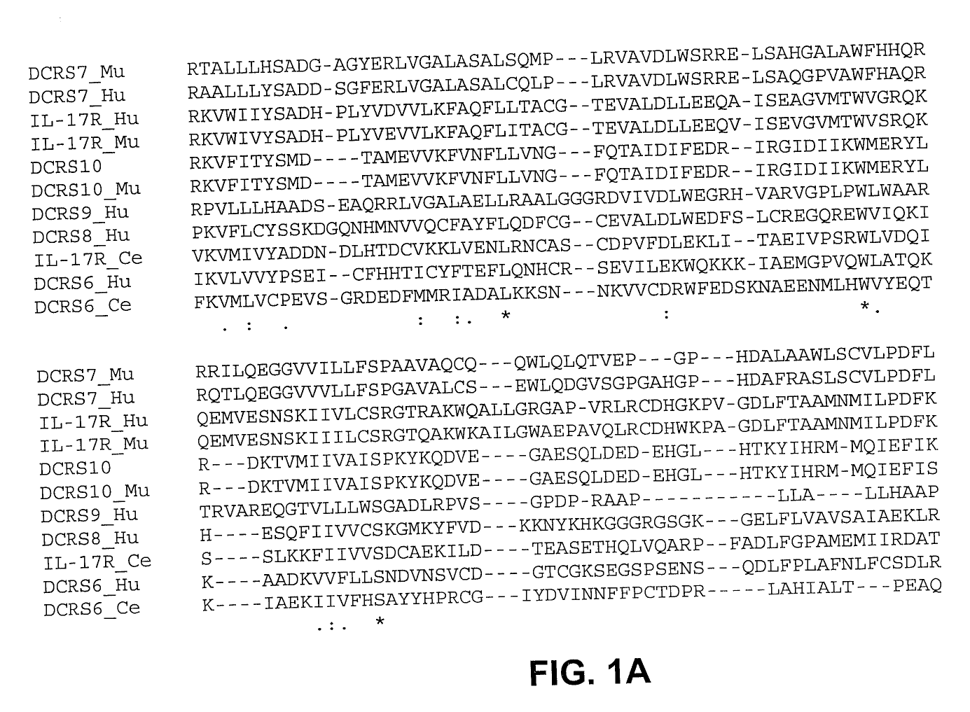

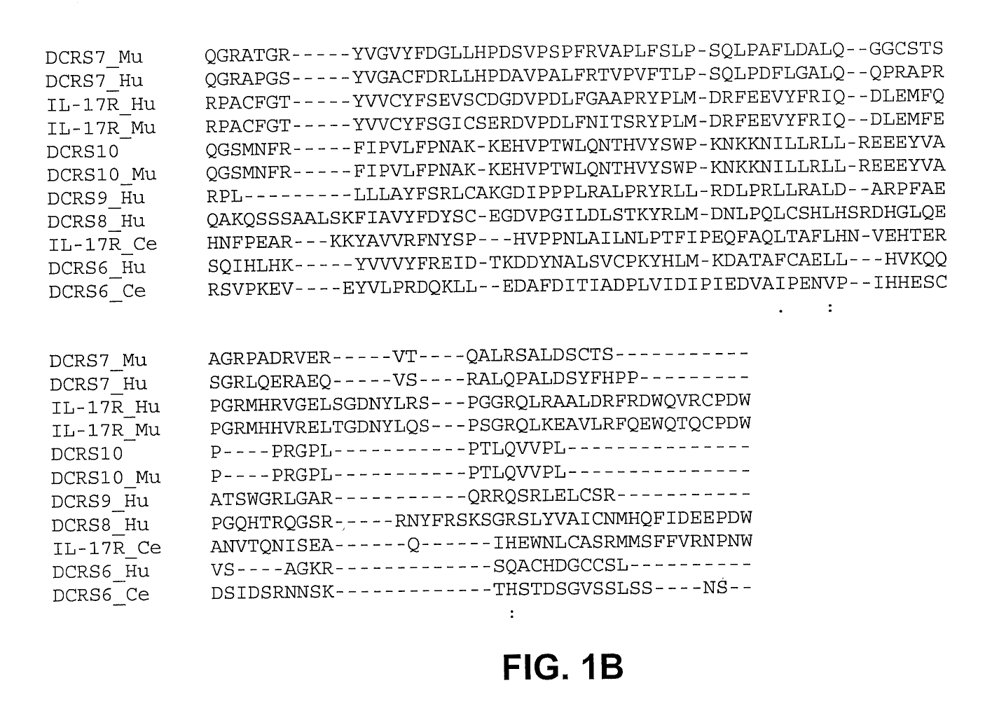

This application claims benefit of U.S. provisional Patent Application No. 60/206,862, filed May 24, 2000.

### FIELD OF THE INVENTION

The present invention relates to compositions and methods for affecting mammalian physiology, including immune system function. In particular, it provides methods to regulate development and/or the immune system. Diagnostic and therapeutic uses of these materials are also disclosed.

### BACKGROUND OF THE INVENTION

Recombinant DNA technology refers generally to techniques of integrating genetic information from a donor source into vectors for subsequent processing, such as through introduction into a host, whereby the transferred genetic information is copied and/or expressed in the new environment. Commonly, the genetic information exists in the form of complementary DNA (cDNA) derived from messenger RNA (MRNA) coding for a desired protein product. The carrier is frequently a plasmid having the capacity to incorporate cDNA for later replication in a host and, in some cases, actually to control expression of the cDNA and thereby direct synthesis of the encoded product in the host. See, e.g., Sambrook, et al. (1989) Molecular Cloning: A Laboratory Manual, (2d ed.) vols. 1-3, CSH Press, N.Y.

For some time, it has been known that the mammalian immune response is based on a series of complex cellular interactions, called the “immune network”. Recent research has provided new insights into the inner workings of this network. While it remains clear that much of the immune response does, in fact, revolve around the network-like interactions of lymphocytes, macrophages, granulocytes, and other cells, immunologists now generally hold the opinion that soluble proteins, known as lymphokines, cytokines, or monokines, play critical roles in controlling these cellular interactions. Thus, there is considerable interest in the isolation, characterization, and mechanisms of action of cell modulatory factors, an understanding of which will lead to significant advancements in the diagnosis and therapy of numerous medical abnormalities, e.g., immune system disorders.

The immune system of vertebrates consists of a number of organs and several different cell types. Two major cell types include the myeloid and lymphoid lineages. Among the lymphoid cell lineage are B cells, which were originally characterized as differentiating in fetal liver or adult bone marrow, and T cells, which were originally characterized as differentiating in the thymus. See, e.g., Paul (ed. 1998) Fundamental hImunology (4th ed.) Raven Press, N.Y.; and Thomson (ed. 1994) The Cytokine Handbook 2d ed., Academic Press, San Diego. Lymphokines apparently mediate cellular activities in a variety of ways. They have been shown to support the proliferation, growth, and/or differentiation of cells, e.g., pluripotential hematopoietic stem cells, into vast numbers of progenitors comprising diverse cellular lineages which make up a complex immune system. Proper and balanced interactions between the cellular components are necessary for a healthy immune response. The different cellular lineages often respond in a different manner when lymphokines are administered in conjunction with other agents.

Cell lineages especially important to the immune response include two classes of lymphocytes: B-cells, which can produce and secrete immunoglobulins (proteins with the capability of recognizing and binding to foreign matter to effect its removal), and T-cells of various subsets that secrete lymphokines and induce or suppress the B-cells and various other cells (including other T-cells) making up the immune network. These lymphocytes interact with many other cell types.

Research to better understand and treat various immune disorders has been hampered by the general inability to maintain cells of the immune system in vitro. Immunologists have discovered that culturing many of these cells can be accomplished through the use of T-cell and other cell supernatants, which contain various growth factors, including many of the lymphokines.

Various growth and regulatory factors exist which modulate morphogenetic development. And many receptors for cytokines are also known. Often there are at least two critical subunits in the fimctional receptor. See, e.g., Gonda and D'Andrea (1997) Blood 89:355-369; Presky, et al. (1996) Proc. Nat'l Acad. Sci. USA 93:14002-14007; Drachman and Kaushansky (1995) Curr. Opin. Hematol. 2:22-28; Theze (1994) Eur. Cvtokine Netw. 5:353-368; and Lemmon and Schlessinger (1994) Trends Biochem. Sci. 19:459-463.

From the foregoing, it is evident that the discovery and development of new soluble proteins and their receptors, including ones similar to lymphokines, should contribute to new therapies for a wide range of degenerative or abnormal conditions which directly or indirectly involve development, differentiation, or function, e.g., of the immune system and/or hematopoietic cells. In particular, the discovery and understanding of novel receptors for lymphokine-like molecules which enhance or potentiate the beneficial activities of other lymphokines would be highly advantageous. However, the lack of understanding of how the immune system is regulated or differentiates has blocked the ability to advantageously modulate the normal defensive mechanisms to biological challenges. Medical conditions characterized by abnormal or inappropriate regulation of the development or physiology of relevant cells thus remain umnanageable. The discovery and characterization of specific cytokines and their receptors will contribute to the development of therapies for a broad range of degenerative or other conditions which affect the immune system, hematopoietic cells, as well as other cell types. The present invention provides new receptors for ligands exhibiting similarity to cytokine like compositions and related compounds, and methods for their use.

### SUMMARY OF THE INVENTION

The present invention is directed to novel receptors related to cytokine receptors, e.g., primate, cytokine receptor like molecular structures, designated DNAX Cytokine Receptor Subunits (DCRS), and their biological activities. In particular, it provides description of various subunits, designated DCRS6, DCRS7, DCRS8, DCRS9, and DCRS IO. Primate, e.g, human, and rodent, e.g., mouse, embodiments of the various subunits are provided. It includes nucleic acids coding for the polypeptides themselves and methods for their production and use. The nucleic acids of the invention are characterized, in part, by their homology to cloned complementary DNA (cDNA) sequences enclosed herein.

The present invention provides a composition of matter selected from: a substantially pure or recombinant polypeptide comprising at least three distinct nonoverlapping segments of at least four amino acids identical to segments of SEQ ID NO: 2, 5, 8, 11, 23, or 26; a substantially pure or recombinant polypeptide comprising at least three distinct nonoverlapping segments of at least four amino acids identical to segments of SEQ ID NO: 14; a substantially pure or recombinant polypeptide comprising at least two distinct nonoverlapping segments of at least five amino acids identical to segments of SEQ ID NO: 14; a natural sequence DCRS8 comprising mature SEQ ID NO: 14; a fusion polypeptide comprising DCRS8 sequence; a substantially pure or recombinant polypeptide comprising at least three distinct nonoverlapping segments of at least four amino acids identical to segments of SEQ ID NO: 17 or 20; a substantially pure or recombinant polypeptide comprising at least two distinct nonoverlapping segments of at least five amino acids identical to segments of SEQ ID NO: 17 or 20; a natural sequence DCRS9 comprising mature SEQ ID NO: 17 or 20; or a fusion polypeptide comprising DCRS9 sequence. Preferably, wherein the distinct nonoverlapping segments of identity include: one of at least eight amino acids; one of at least four amino acids and a second of at least five amino acids; at least three segments of at least four, five, and six amino acids, or one of at least twelve amino acids. In other embodiments, the: polypeptide: comprises a mature sequence of Tables 1, 2, 3, 4, or 5; is an unglycosylated form of DCRS8 or DCRS9; is from a primate, such as a human; comprises at least seventeen amino acids of SEQ ID NO: 14 or 17; exhibits at least four nonoverlapping segments of at least seven amino acids of SEQ ID NO: 14 or 17; is a natural allelic variant of DCRS8 or DCRS9; has a length at least about 30 amino acids; exhibits at least two non-overlapping epitopes which are specific for a primate DCRS8 or DCRS9; is glycosylated; has a molecular weight of at least 30 kD with natural glycosylation; is a synthetic polypeptide; is attached to a solid substrate; is conjugated to another chemical moiety; is a 5-fold or less substitution from natural sequence; or is a deletion or insertion variant from a natural sequence.

The invention further embraces a composition comprising: a substantially pure DCRS8 or DCRS9 and another cytokine receptor family member; a sterile DCRS8 or DCRS9 polypeptide; the DCRS8 or DCRS9 polypeptide and a carrier, wherein the carrier is: an aqueous compound, including water, saline, and/or buffer; and/or formulated for oral, rectal, nasal, topical, or parenteral administration. Additional embodiments include a polypeptide comprising: mature protein sequence of Tables 1, 2, 3, 4, or 5; a detection or purification tag, including a FLAG, His6, or Ig sequence; or sequence of another cytokine receptor protein. Kit embodiments include ones comprising a described polypeptide, and: a compartment comprising the protein or polypeptide; or instructions for use or disposal of reagents in the kit.

Binding compositions are provided, e.g., comprising an antigen binding site from an antibody, which specifically binds to a natural DCRS8 or DCRS9 polypeptide, wherein: the binding compound is in a container; the DCRS8 or DCRS9 polypeptide is from a human; the binding compound is an Fv, Fab, or Fab2 fragment; the binding compound is conjugated to another chemical moiety; or the antibody: is raised against a peptide sequence of a mature polypeptide of Table 3 or 4; is raised against a mature DCRS8 or DCRS9; is raised to a purified human DCRS8 or DCRS9; is immunoselected; is a polyclonal antibody; binds to a denatured DCRS8 or DCRS9; exhibits a Kd to antigen of at least 30 lM; is attached to a solid substrate, including a bead or plastic membrane; is in a sterile composition; or is detectably labeled, including a radioactive or fluorescent label. Kits include ones comprising such a binding compound, and: a compartment comprising the binding compound; or instructions for use or disposal of reagents in the kit.

The invention also provides methods of producing an antigen:antibody complex, comprising contacting under appropriate conditions a primate DCRS8 or DCRS9 polypeptide with a described antibody, thereby allowing the complex to form. Preferred methods include ones wherein: the complex is purified from other cytokine receptors; the complex is purified from other antibody; the contacting is with a sample comprising an interferon; the contacting allows quantitative detection of the antigen; the contacting is with a sample comprising the antibody; or the contacting allows quantitative detection of the antibody. Further compositions include those comprising: a sterile binding compound, as described, or the binding compound and a carrier, wherein the carrier is: an aqueous compound, including water, saline, and/or buffer; and/or formulated for oral, rectal, nasal, topical, or parenteral administration.

Nucleic acid compositions include an isolated or recombinant nucleic acid encoding a desribed polypeptide wherein the: DCRS8 or DCRS9 is from a human; or the nucleic acid: encodes an antigenic peptide sequence of Table 3 or 4; encodes a plurality of antigenic peptide sequences of Table 3 or 4; exhibits identity over at least thirteen nucleotides to a natural cDNA encoding the segment; is an expression vector; further comprises an origin of replication; is from a natural source; comprises a detectable label; comprises synthetic nucleotide sequence; is less than 6 kb, preferably less than 3 kb; is from a primate; comprises a natural full length coding sequence; is a hybridization probe for a gene encoding the DCRS8 or DCRS9; or is a PCR primer, PCR product, or mutagenesis primer. Also provided are a cell or tissue comprising such a recombinant nucleic acid, e.g., where the cell is: a prokaryotic cell; a eukaryotic cell; a bacterial cell; a yeast cell; an insect cell; a mammalian cell; a mouse cell; a primate cell; or a human cell.

Kit embodiments include those comprising a described nucleic acid and: a compartment comprising the nucleic acid; a compartment further comprising a primate DCRS8 or DCRS9 polypeptide; or instructions for use or disposal of reagents in the kit.

Other nucleic acids provided include ones which: hybridize under wash conditions of 30 minutes at 30° C and less than 2M salt to the coding portion of SEQ ID NO: 13 or 16; or exhibit identity over a stretch of at least about 30 nucleotides to a primate DCRS8 or DCRS9. Preferably, such will be nucleic acids where: the wash conditions are: at 45° C and/or 500 mM salt; at 55° C and/or 150 mM salt; or the stretch is at least 55 or 75 nucleotides.

Also provided are methods of modulating physiology or development of a cell or tissue culture cells comprising contacting the cell with an agonist or antagonist of a mammalian DCRS8 or DCRS9. Preferably, the cell is transformed with a nucleic acid encoding the DCRS8 or DCRS9 and another cytokine receptor subunit.

### DETAILED DESCRIPTION OF THE PREFERRED EMBODIMENTS

### OUTLINE

I. General

II. Activities

III. Nucleic acids

A. encoding fragments, sequence, probes

B. mutations, chimeras, fusions

C. making nucleic acids

D. vectors, cells comprising

IV. Proteins, Peptides

A. fragments, sequence, immunogens, antigens

B. muteins

C. agonists/antagonists, finctional equivalents

D. making proteins

V. Making nucleic acids, proteins

A. synthetic

B. recombinant

C. natural sources

VI. Antibodies

A. polyclonals

B. monoclonal

C. fragments; Kd

D. anti-idiotypic antibodies

E. hybridoma cell lines

VII. Kits and Methods to quantify DCRSs

A. ELISA

B. assay MRNA encoding

C. qualitative/quantitative

D. kits

VIII. Therapeutic compositions, methods

A. combination compositions

B. unit dose

C. administration

IX. Screening

X. Ligands

### I. General

The present invention provides the amino acid sequence and DNA sequence of mammalian, herein primate, cytokine receptor-like subunit molecules, these designated DNAX Cytokine Receptor Subunits 6 (DCRS6), 7 (DCRS7), 8 (DCRS8), 9 (DCRS9), and 10 (DCRS1O) having particular defined properties, both structural and biological. Various cDNAs encoding these molecules were obtained from primate, e.g., human, and/or rodent, e.g., mouse, cDNA sequence libraries. Other primate or other mammalian counterparts would also be desired.

Some of the standard methods applicable are described or referenced, e.g., in Maniatis, et al. (1982) Molecular Cloning A Laboratorv Manual, Cold Spring Harbor Laboratory, Cold Spring Harbor Press; Sambrook, et al. (1989) Molecular Cloning: A Laboratorv Manual, (2d ed.), vols. 1-3, CSH Press, NY; Ausubel, et al., Biology, Greene Publishing Associates, Brooklyn, NY; or Ausubel, et al. (1987 and periodic supplements) Current Protocols in Molecular Biology, Greene/Wiley, New York; each of which is incorporated herein by reference.

Nucleotide (SEQ ID NO: 1) and corresponding amino acid sequence (SEQ ID NO: 2) of a primate, e.g., human, DCRS6 coding segment is shown in Table 1 along with reverse translation (SEQ ID NO: 3). Rodent, e.g., mouse, counterpart sequences are provided, e.g., SEQ ID NO: 4-6.

Similarly, nucleotide (SEQ ID NO: 7) and corresponding amino acid sequence (SEQ ID NO: 8) of a primate, e.g., human, DCRS7 coding segment is shown in Table 2 along with reverse translation (SEQ ID NO: 9). Rodent, e.g., mouse, counterpart sequences are provided, e.g., SEQ ID NO: 10-12. Nucleotide (SEQ ID NO: 13) and corresponding amino acid sequence (SEQ ID NO: 14) of a primate, e.g., human, DCRS8 coding segment is shown in Table 3 along with reverse translation (SEQ ID NO: 15).

Nucleotide (SEQ ID NO: 16) and corresponding amino acid sequence (SEQ ID NO: 17) of a primate, e.g., human, DCRS9 coding segment is shown in Table 4 along with reverse translation (SEQ ID NO: 18). Rodent, e.g., mouse, counterpart sequences are provided, e.g., SEQ ID NO: 19-21. Nucleotide (SEQ ID NO: 22) and corresponding amino acid sequence (SEQ ID NO: 23) of a primate, e.g., human, DCRS10 coding segment is shown in Table 5 along with reverse translation (SEQ ID NO: 24). Rodent, e.g., mouse, counterpart sequences are provided, e.g., SEQ ID NO: 26-27. Table 1: Nucleotide and polypeptide sequences of DNAX Cytokine Receptor Subunit like embodiments (DCRS6). Primate, e.g., human, embodiment (see SEQ ID NO: 1 and 2). Predicted signal sequence indicated, but may vary by a few positions and depending upon cell type.

| | |
|---|---|
| gcg atg tcg ctc gtg ctg cta agc ctg gcc gcg ctg tgc agg agc gcc | 48 |
| Met Ser Leu Val Leu Leu Ser Leu Ala Ala Leu Cys Arg Ser Ala | |
| −10 −5 −1 1 | |
| gta ccc cga gag ccg acc gtt caa tgt ggc tct gaa act ggg cca tct | 96 |
| Val Pro Arg Glu Pro Thr Val Gln Cys Gly Ser Glu Thr Gly Pro Ser | |
| 5 10 15 | |
| cca gag tgg atg cta caa cat gat cta atc ccg gga gac ttg agg gac | 144 |
| Pro Glu Trp Met Leu Gln His Asp Leu Ile Pro Gly Asp Leu Arg Asp | |
| 20 25 30 | |
| ctc cga gta gaa cct gtt aca act agt gtt gca aca ggg gac tat tca | 192 |
| Leu Arg Val Glu Pro Val Thr Thr Ser Val Ala Thr Gly Asp Tyr Ser | |
| 35 40 45 | |
| att ttg atg aat gta agc tgg gta ctc cgg gca gat gcc agc atc cgc | 240 |
| Ile Leu Met Asn Val Ser Trp Val Leu Arg Ala Asp Ala Ser Ile Arg | |
| 50 55 60 65 | |
| ttg ttg aag gcc acc aag att tgt gtg acg ggc aaa agc aac ttc cag | 288 |
| Leu Leu Lys Ala Thr Lys Ile Cys Val Thr Gly Lys Ser Asn Phe Gln | |
| 70 75 80 | |
| tcc tac agc tgt gtg agg tgc aat tac aca gag gcc ttc cag act cag | 336 |
| Ser Tyr Ser Cys Val Arg Cys Asn Tyr Thr Glu Ala Phe Gln Thr Gln | |
| 85 90 95 | |
| acc aga ccc tct ggt ggt aaa tgg aca ttt tcc tat atc ggc ttc cct | 384 |
| Thr Arg Pro Ser Gly Gly Lys Trp Thr Phe Ser Tyr Ile Gly Phe Pro | |
| 100 105 110 | |
| gta gag ctg aac aca gtc tat ttc att ggg gcc cat aat att cct aat | 432 |
| Val Glu Leu Asn Thr Val Tyr Phe Ile Gly Ala His Asn Ile Pro Asn | |
| 115 120 125 | |
| gca aat atg aat gaa gat ggc cct tcc atg tct gtg aat ttc acc tca | 480 |
| Ala Asn Met Asn Glu Asp Gly Pro Ser Met Ser Val Asn Phe Thr Ser | |
| 130 135 140 145 | |
| cca ggc tgc cta gac cac ata atg aaa tat aaa aaa aag tgt gtc aag | 528 |
| Pro Gly Cys Leu Asp His Ile Met Lys Tyr Lys Lys Lys Cys Val Lys | |
| 150 155 160 | |
| gcc gga agc ctg tgg gat ccg aac atc act gct tgt aag aag aat gag | 576 |
| Ala Gly Ser Leu Trp Asp Pro Asn Ile Thr Ala Cys Lys Lys Asn Glu | |
| 165 170 175 | |
| gag aca gta gaa gtg aac ttc aca acc act ccc ctg gga aac aga tac | 624 |
| Glu Thr Val Glu Val Asn Phe Thr Thr Thr Pro Leu Gly Asn Arg Tyr | |
| 180 185 190 | |
| atg gct ctt atc caa cac agc act atc atc ggg ttt tct cag gtg ttt | 672 |
| Met Ala Leu Ile Gln His Ser Thr Ile Ile Gly Phe Ser Gln Val Phe | |
| 195 200 205 | |
| gag cca cac cag aag aaa caa acg cga gct tca gtg gtg att cca gtg | 720 |
| Glu Pro His Gln Lys Lys Gln Thr Arg Ala Ser Val Val Ile Pro Val | |
| 210 215 220 225 | |
| act ggg gat agt gaa ggt gct acg gtg cag ctg act cca tat ttt cct | 768 |
| Thr Gly Asp Ser Glu Gly Ala Thr Val Gln Leu Thr Pro Tyr Phe Pro | |
| 230 235 240 | |
| act tgt ggc agc gac tgc atc cga cat aaa gga aca gtt gtg ctc tgc | 816 |
| Thr Cys Gly Ser Asp Cys Ile Arg His Lys Gly Thr Val Val Leu Cys | |
| 245 250 255 | |
| cca caa aca ggc gtc cct ttc cct ctg gat aac aac aaa agc aag ccg | 864 |
| Pro Gln Thr Gly Val Pro Phe Pro Leu Asp Asn Asn Lys Ser Lys Pro | |
| 260 265 270 | |
| gga ggc tgg ctg cct ctc ctc ctg ctg tct ctg ctg gtg gcc aca tgg | 912 |
| Gly Gly Trp Leu Pro Leu Leu Leu Leu Ser Leu Leu Val Ala Thr Trp | |
| 275 280 285 | |
| gtg ctg gtg gca ggg atc tat cta atg tgg agg cac gaa agg atc aag | 960 |
| Val Leu Val Ala Gly Ile Tyr Leu Met Trp Arg His Glu Arg Ile Lys | |
| 290 295 300 305 | |
| aag act tcc ttt tct acc acc aca cta ctg ccc ccc att aag gtt ctt | 1008 |
| Lys Thr Ser Phe Ser Thr Thr Thr Leu Leu Pro Pro Ile Lys Val Leu | |
| 310 315 320 | |
| gtg gtt tac cca tct gaa ata tgt ttc cat cac aca att tgt tac ttc | 1056 |
| Val Val Tyr Pro Ser Glu Ile Cys Phe His His Thr Ile Cys Tyr Phe | |
| 325 330 335 | |
| act gaa ttt ctt caa aac cat tgc aga agt gag gtc atc ctt gaa aag | 1104 |
| Thr Glu Phe Leu Gln Asn His Cys Arg Ser Glu Val Ile Leu Glu Lys | |
| 340 345 350 | |
| tgg cag aaa aag aaa ata gca gag atg ggt cca gtg cag tgg ctt gcc | 1152 |
| Trp Gln Lys Lys Lys Ile Ala Glu Met Gly Pro Val Gln Trp Leu Ala | |
| 355 360 365 | |
| act caa aag aag gca gca gac aaa gtc gtc ttc ctt ctt tcc aat gac | 1200 |
| Thr Gln Lys Lys Ala Ala Asp Lys Val Val Phe Leu Leu Ser Asn Asp | |
| 370 375 380 385 | |
| gtc aac agt gtg tgc gat ggt acc tgt ggc aag agc gag ggc agt ccc | 1248 |
| Val Asn Ser Val Cys Asp Gly Thr Cys Gly Lys Ser Glu Gly Ser Pro | |
| 390 395 400 | |
| agt gag aac tct caa gac ctc ttc ccc ctt gcc ttt aac ctt ttc tgc | 1296 |
| Ser Glu Asn Ser Gln Asp Leu Phe Pro Leu Ala Phe Asn Leu Phe Cys | |
| 405 410 415 | |
| agt gat cta aga agc cag att cat ctg cac aaa tac gtg gtg gtc tac | 1344 |
| Ser Asp Leu Arg Ser Gln Ile His Leu His Lys Tyr Val Val Val Tyr | |
| 420 425 430 | |
| ttt aga gag att gat aca aaa gac gat tac aat gct ctc agt gtc tgc | 1392 |
| Phe Arg Glu Ile Asp Thr Lys Asp Asp Tyr Asn Ala Leu Ser Val Cys | |
| 435 440 445 | |
| ccc aag tac cac ctc atg aag gat gcc act gct ttc tgt gca gaa ctt | 1440 |
| Pro Lys Tyr His Leu Met Lys Asp Ala Thr Ala Phe Cys Ala Glu Leu | |
| 450 455 460 465 | |
| ctc cat gtc aag cag cag gtg tca gca gga aaa aga tca caa gcc tgc | 1488 |
| Leu His Val Lys Gln Gln Val Ser Ala Gly Lys Arg Ser Gln Ala Cys | |
| 470 475 480 | |
| cac gat ggc tgc tgc tcc ttg tagcccaccc atgagaagca agagacctta | 1539 |
| His Asp Gly Cys Cys Ser Leu | |
| 485 | |
| aaggcttcct atcccaccaa ttacagggaa aaaacgtgtg atgatcctga agcttactat | 1599 |
| gcagcctaca aacagcctta gtaattaaaa cattttatac caataaaatt ttcaaatatt | 1659 |
| gctaactaat gtagcattaa ctaacgattg gaaactacat ttacaacttc aaagctgttt | 1719 |
| tatacataga aatcaattac agctttaatt gaaaactgta accattttga taatgcaaca | 1779 |
| ataaagcatc ttcagcc | 1796 |

MSLVLLSLAALCRSAVPREPTVQCGSETGPSPEWMLQHDLIPGDLRDLRVEPVTTSVATGDYSILMNVSWVL
RADASIRLLKATKICVTGKSNFQSYSCVRCNYTEAFQTQTRPSGGKWTFSYIGFPVELNTVYFIGAHNIPNA
NMNEDGPSMSVNFTSPGCLDHIMKYKKKCVKAGSLWDPNITACKKNEETVEVNFTTTPLGNRYMALIQHSTI
IGFSQVFEPHQKKQTRASVVIPVTGDSEGATVQLTPYFPTCGSDCIRHKGTVVLCPQTGVPFPLDNNKSKPG
GWLPLLLLSLLVATWVLVAGIYLMWRHERIKKTSFSTTTLLPPIKVLVVYPSEICFHHTICYFTEFLQNHCR
SEVILEKWQKKKIAEMGPVQWLATQKKAADKVVFLLSNDVNSVCDGTCGKSEGSPSENSQDLFPLAFNLFCS
DLRSQIHLHKYVVVYFREIDTKDDYNALSVCPKYHLMKDATAFCAELLHVKQQVSAGKRSQACHDGCCSL.

| Reverse translation of primate, e.g., human, DCRS6 (SEQ ID NO:3): | |
|---|---|
| atgwsnytng tnytnytnws nytngcngcn ytntgymgnw sngcngtncc nmgngarccn | 60 |
| acngtncart gyggnwsnga racnggnccn wsnccngart ggatgytnca rcaygayytn | 120 |
| athccnggng ayytnmgnga yytnmgngtn garccngtna cnadnwsngt ngcnacnggn | 180 |
| gaytaywsna thytnatgaa ygtnwsntgg gtnytnmgng cngaygcnws nathmgnytn | 240 |
| ytnaargcna cnaarathtg ygtnacnggn aarwsnaayt tycarwsnta ywsntgygtn | 300 |
| mgntgyaayt ayacngargc nttycaracn caracnmgnc cnwsnggngg naartggacn | 360 |
| ttywsntaya thggnttycc ngtngarytn aayacngtnt ayttyathgg ngcncayaay | 420 |
| athccnaayg cnaayatgaa ygargayggn ccnwsnatgw sngtnaaytt yacnwsnccn | 480 |
| ggntgyytng aycayathat gaartayaar aaraartgyg tnaargcngg nwsnytntgg | 540 |
| gayccnaaya thacngcntg yaaraaraay gargaracng tngargtnaa yttyacnacn | 600 |
| acnccnytng gnaaymgnta yatggcnytn athcarcayw snacnathat hggnttywsn | 660 |
| cargtnttyg arccncayca raaraarcar acnmgngcnw sngtngtnat hccngtnacn | 720 |
| ggngaywsng arggngcnac ngtncarytn acnccntayt tyccnacntg yggnwsngay | 780 |
| tgyathmgnc ayaarggnac ngtngtnytn tgyccncara cnggngtncc nttyccnytn | 840 |
| gayaayaaya arwsnaarcc nggnggntgg ytnccnytny tnytnytnws nytnytngtn | 900 |
| gcnacntggg tnytngtngc nggnathtay ytnatgtggm gncaygarmg nathaaraar | 960 |
| acnwsnttyw snacnacnac nytnytnccn ccnathaarg tnytngtngt ntayccnwsn | 1020 |
| garathtgyt tycaycayac nathtgytay ttyacngart tyytncaraa ycaytgymgn | 1080 |
| wsngargtna thytngaraa rtggcaraar aaraarathg cngaratggg nccngtncar | 1140 |
| tggytngcna cncaraaraa rgcngcngay aargtngtnt tyytnytnws naaygaygtn | 1200 |
| aaywsngtnt gygayggnac ntgyggnaar wsngarggnw snccnwsnga raaywsncar | 1260 |
| gayytnttyc cnytngcntt yaayytntty tgywsngayy tnmgnwsnca rathcayytn | 1320 |
| cayaartayg tngtngtnta yttymgngar athgayacna argaygayta yaaygcnytn | 1380 |
| wsngtntgyc cnaartayca yytnatgaar gaygcnacng cnttytgygc ngarytnytn | 1440 |
| caygtnaarc arcargtnws ngcnggnaar mgnwsncarg cntgycayga yggntgytgy | 1500 |
| wsnytn | 1506 |

| Rodent, e.g., mouse embodiment. (see SEQ ID NO:4 and 5) | |
|---|---|
| gat ttc agc agc cag acg cat ctg cac aaa tac ctg gag gtc tat ctt | 48 |
| Asp Phe Ser Ser Gln Thr His Leu His Lys Tyr Leu Glu Val Tyr Leu | |
| 1 5 10 15 | |
| ggg gga gca gac ctc aaa ggc gac tat aat gcc ctg agt gtc tgc ccc | 96 |
| Gly Gly Ala Asp Leu Lys Gly Asp Tyr Asn Ala Leu Ser Val Cys Pro | |
| 20 25 30 | |
| caa tat cat ctc atg aag gac gcc aca gct ttc cac aca gaa ctt ctc | 144 |
| Gln Tyr His Leu Met Lys Asp Ala Thr Ala Phe His Thr Glu Leu Leu | |
| 35 40 45 | |
| aag gct acg cag agc atg tca gtg aag aaa cgc tca caa gcc tgc cat | 192 |
| Lys Ala Thr Gln Ser Met Ser Val Lys Lys Arg Ser Gln Ala Cys His | |
| 50 55 60 | |
| gat agc tgt tca ccc ttg tagtccaccc gggggaatag agactctgaa | 240 |
| Asp Ser Cys Ser Pro Leu | |
| 65 70 | |
| gccttcctac tctcccttcc agtgacaaat gctgtgtgac gactctgaaa tgtgtgggag | 300 |
| aggctgtgtg gaggtagtgc tatgtacaaa cttgctttaa aactggagtt tgcaaagtca | 360 |
| acctgagcat acacgcctga ggctagtcat tggctggatt tatgaagaca acacagttac | 420 |
| agacaataat gagtgggacc tacatttggg atatacccaa agctgggtaa tgattatcac | 480 |
| tgagaaccac gcactctggc catgaggtaa tacggcactt ccctgtcagg ctgtctgtca | 540 |
| ggttgggtct gtcttgcact gcccatgctc tatgctgcac gtagaccgtt ttgtaacatt | 600 |
| taatctgtt aatgaataat ccgtttggga ggctctc | 637 |

DFSSQTHLHKYLEVYLGGDLKGDYNALSVCPQYHLMKDATAFHTELLKATQSMSVKKRSQACHDSCSPL

| | |
|---|---|
| Reverse translation of rodent, e.g., mouse, DCRS6: (SEQ ID NO:6) | |
| gayttywsnw sncaracnca yytncayaar tayytngarg tntayytngg nggngcngay | 60 |
| ytnaarggng aytayaaygc nytnwsngtn tgyccncart aycayytnat gaargaygcn | 120 |
| acngcnttyc ayacngaryt nytnaargcn acncarwsna tgwsngtnaa raarmgnwsn | 180 |
| cargcntgyc aygaywsntg ywsnccnytn | 210 |

TABLE 2
Nucleotide and polypeptide sequences of DNAX Cytokine Receptor
Subunit like embodiments (DCRS7). Primate, e.g., human, embodiment
(see SEQ ID NO:7 and 8). Predicted signal sequence indicated, but
may vary by a few positons and depending upon cell type.

| | |
|---|---|
| gagtcaggac tcccaggaca gagagtgcac aaactaccca gcacagcccc ctccgccccc | 60 |
| tctggaggct gaagagggat tccagcccct gccacccaca gacacgggct gactggggtg | 120 |
| tctgcccccc ttgggggcan ccacagggcc tcaggcctgg gtgccacctg gcactagaag | 180 |
| atg cct gtg ccc tgg ttc ttg ctg tcc ttg gca ctg ggc cga agc cag | 228 |
| Met Pro Val Pro Trp Phe Leu Leu Ser Leu Ala Leu Gly Arg Ser Gln | |
| −20 −15 − −5 | |
| tgg atc ctt tct ctg gag agg ctt gtg ggg cct cag gac gct acc cac | 276 |
| Trp Ile Leu Ser Leu Glu Arg Leu Val Gly Pro Gln Asp Ala Thr His | |
| −1 1 5 10 | |
| tgc tct ccg ggc ctc tcc tgc cgc ctc tgg gac agt gac ata ctc tgc | 324 |
| Cys Ser Pro Gly Leu Ser Cys Arg Leu Trp Asp Ser Asp Ile Leu Cys | |
| 15 20 25 | |
| ctg cct ggg gac atc gtg cct gct ccg ggc ccc gtg ctg gcg cct acg | 372 |
| Leu Pro Gly Asp Ile Val Pro Ala Pro Gly Pro Val Leu Ala Pro Thr | |
| 30 35 40 | |
| cac ctg cag aca gag ctg gtg ctg agg tgc cag aag gag acc gac tgt | 420 |
| His Leu Gln Thr Glu Leu Val Leu Arg Cys Gln Lys Glu Thr Asp Cys | |
| 45 50 55 60 | |
| gac ctc tgt ctg cgt gtg gct gtc cac ttg gcc gtg cat ggg cac tgg | 468 |
| Asp Leu Cys Leu Arg Val Ala Val His Leu Ala Val His Gly His Trp | |
| 65 70 75 | |
| gaa gag cct gaa gat gag gaa aag ttt gga gga gca gct gac tta ggg | 516 |
| Glu Glu Pro Glu Asp Glu Glu Lys Phe Gly Gly Ala Ala Asp Leu Gly | |
| 80 85 90 | |
| gtg gag gag cct agg aat gcc tct ctc cag gcc caa gtc gtg ctc tcc | 564 |
| Val Glu Glu Pro Arg Asn Ala Ser Leu Gln Ala Gln Val Val Leu Ser | |
| 95 100 105 | |
| ttc cag gcc tac cct act gcc cgc tgc gtc ctg ctg gag gtg caa gtg | 612 |
| Phe Gln Ala Tyr Pro Thr Ala Arg Cys Val Leu Leu Glu Val Gln Val | |
| 110 115 120 | |
| cct gct gcc ctt gtg cag ttt ggt cag tct gtg ggc tct gtg gta tat | 660 |
| Pro Ala Ala Leu Val Gln Phe Gly Gln Ser Val Gly Ser Val Val Tyr | |
| 125 130 135 140 | |
| gac tgc ttc gag gct gcc cta ggg agt gag gta cga atc tgg tcc tat | 708 |
| Asp Cys Phe Glu Ala Ala Leu Gly Ser Glu Val Arg Ile Trp Ser Tyr | |
| 145 150 155 | |
| act cag ccc agg tac gag aag gaa ctc aac cac aca cag cag ctg cct | 756 |
| Thr Gln Pro Arg Tyr Glu Lys Glu Leu Asn His Thr Gln Gln Leu Pro | |
| 160 165 170 | |
| gac tgc agg ggg ctc gaa gtc tgg aac agc atc ccg agc tgc tgg gcc | 804 |
| Asp Cys Arg Gly Leu Glu Val Trp Asn Ser Ile Pro Ser Cys Trp Ala | |
| 175 180 185 | |
| ctg ccc tgg ctc aac gtg tca gca gat ggt gac aac gtg cat ctg gtt | 852 |
| Leu Pro Trp Leu Asn Val Ser Ala Asp Gly Asp Asn Val His Leu Val | |
| 190 195 200 | |
| ctg aat gtc tct gag gag cag cac ttc ggc ctc tcc ctg tac tgg aat | 900 |
| Leu Asn Val Ser Glu Glu Gln His Phe Gly Leu Ser Leu Tyr Trp Asn | |
| 205 210 215 220 | |
| cag gtc cag ggc ccc cca aaa ccc cgg tgg cac aaa aac ctg act gga | 948 |
| Gln Val Gln Gly Pro Pro Lys Pro Arg Trp His Lys Asn Leu Thr Gly | |
| 225 230 235 | |
| ccg cag atc att acc ttg aac cac aca gac ctg gtt ccc tgc ctc tgt | 996 |
| Pro Gln Ile Ile Thr Leu Asn His Thr Asp Leu Val Pro Cys Leu Cys | |
| 240 245 250 | |
| att cag gtg tgg cct ctg gaa cct gac tcc gtt agg acg aac atc tgc | 1044 |
| Ile Gln Val Trp Pro Leu Glu Pro Asp Ser Val Arg Thr Asn Ile Cys | |
| 255 260 265 | |
| ccc ttc agg gag gac ccc cgc gca cac cag aac ctc tgg caa gcc gcc | 1092 |
| Pro Phe Arg Glu Asp Pro Arg Ala His Gln Asn Leu Trp Gln Ala Ala | |
| 270 275 280 | |
| cga ctg cga ctg ctg acc ctg cag agc tgg ctg ctg gac gca ccg tgc | 1140 |
| Arg Leu Arg Leu Leu Thr Leu Gln Ser Trp Leu Leu Asp Ala Pro Cys | |
| 285 290 295 300 | |
| tcg ctg ccc gca gaa gcg gca ctg tgc tgg cgg gct ccg ggt ggg gac | 1188 |
| Ser Leu Pro Ala Glu Ala Ala Leu Cys Trp Arg Ala Pro Gly Gly Asp | |
| 305 310 315 | |
| ccc tgc cag cca ctg gtc cca ccg ctt tcc tgg gag aat gtc act gtg | 1236 |
| Pro Cys Gln Pro Leu Val Pro Pro Leu Ser Trp Glu Asn Val Thr Val | |
| 320 325 330 | |
| gac gtg aac agc tcg gag aag ctg cag ctg cag gag tgc ttg tgg gct | 1284 |
| Asp Val Asn Ser Ser Glu Lys Leu Gln Leu Gln Glu Cys Leu Trp Ala | |
| 335 340 345 | |
| gac tcc ctg ggg cct ctc aaa gac gat gtg cta ctg ttg gag aca cga | 1332 |
| Asp Ser Leu Gly Pro Leu Lys Asp Asp Val Leu Leu Leu Glu Thr Arg | |
| 350 355 360 | |
| ggc ccc cag gac aac aga tcc ctc tgt gcc ttg gaa ccc agt ggc tgt | 1380 |
| Gly Pro Gln Asp Asn Arg Ser Leu Cys Ala Leu Glu Pro Ser Gly Cys | |
| 365 370 375 380 | |
| act tca cta ccc agc aaa gcc tcc acg agg gca gct cgc ctt gga gag | 1428 |
| Thr Ser Leu Pro Ser Lys Ala Ser Thr Arg Ala Ala Arg Leu Gly Glu | |
| 385 390 395 | |
| tac tta cta caa gac ctg cag tca ggc cag tgt ctg cag cta tgg gac | 1476 |
| Tyr Leu Leu Gln Asp Leu Gln Ser Gly Gln Cys Leu Gln Leu Trp Asp | |
| 400 405 410 | |
| gat gac ttg gga gcg cta tgg gcc tgc ccc atg gac aaa tac atc cac | 1524 |
| Asp Asp Leu Gly Ala Leu Trp Ala Cys Pro Met Asp Lys Tyr Ile His | |
| 415 420 425 | |
| aag cgc tgg gcc ctc gtg tgg ctg gcc tgc cta ctc ttt gcc gct gcg | 1572 |
| Lys Arg Trp Ala Leu Val Trp Leu Ala Cys Leu Leu Phe Ala Ala Ala | |
| 430 435 440 | |
| ctt tcc ctc atc ctc ctt ctc aaa aag gat cac gcg aaa ggg tgg ctg | 1620 |
| Leu Ser Leu Ile Leu Leu Leu Lys Lys Asp His Ala Lys Gly Trp Leu | |
| 445 450 455 460 | |
| agg ctc ttg aaa cag gac gtc cgc tcg ggg gcg gcc gcc agg ggc cgc | 1668 |
| Arg Leu Leu Lys Gln Asp Val Arg Ser Gly Ala Ala Ala Arg Gly Arg | |
| 465 470 475 | |
| gcg gct ctg ctc ctc tac tca gcc gat gac tcg ggt ttc gag cgc ctg | 1716 |
| Ala Ala Leu Leu Leu Tyr Ser Ala Asp Asp Ser Gly Phe Glu Arg Leu | |
| 480 485 490 | |
| gtg ggc gcc ctg gcg tcg gcc ctg tgc cag ctg ccg ctg cgc gtg gcc | 1764 |
| Val Gly Ala Leu Ala Ser Ala Leu Cys Gln Leu Pro Leu Arg Val Ala | |
| 495 500 505 | |
| gta gac ctg tgg agc cgt cgt gaa ctg agc gcg cag ggg ccc gtg gct | 1812 |
| Val Asp Leu Trp Ser Arg Arg Glu Leu Ser Ala Gln Gly Pro Val Ala | |
| 510 515 520 | |
| tgg ttt cac gcg cag cgg cgc cag acc ctg cag gag ggc ggc gtg gtg | 1860 |
| Trp Phe His Ala Gln Arg Arg Gln Thr Leu Gln Glu Gly Gly Val Val | |
| 525 530 535 540 | |
| gtc ttg ctc ttc tct ccc ggt gcg gtg gcg ctg tgc agc gag tgg cta | 1908 |
| Val Leu Leu Phe Ser Pro Gly Ala Val Ala Leu Cys Ser Glu Trp Leu | |
| 545 550 555 | |
| cag gat ggg gtg tcc ggg ccc ggg gcg cac ggc ccg cac gac gcc ttc | 1956 |
| Gln Asp Gly Val Ser Gly Pro Gly Ala His Gly Pro His Asp Ala Phe | |
| 560 565 570 | |
| cgc gcc tcg ctc agc tgc gtg ctg ccc gac ttc ttg cag ggc cgg gcg | 2004 |
| Arg Ala Ser Leu Ser Cys Val Leu Pro Asp Phe Leu Gln Gly Arg Ala | |
| 575 580 585 | |
| ccc ggc agc tac gtg ggg gcc tgc ttc gac agg ctg ctc cac ccg gac | 2052 |
| Pro Gly Ser Tyr Val Gly Ala Cys Phe Asp Arg Leu Leu His Pro Asp | |
| 590 595 600 | |
| gcc gta ccc gcc ctt ttc cgc acc gtg ccc gtc ttc aca ctg ccc tcc | 2100 |
| Ala Val Pro Ala Leu Phe Arg Thr Val Pro Val Phe Thr Leu Pro Ser | |
| 605 610 615 620 | |
| caa ctg cca gac ttc ctg ggg gcc ctg cag cag cct cgc gcc ccg cgt | 2148 |
| Gln Leu Pro Asp Phe Leu Gly Ala Leu Gln Gln Pro Arg Ala Pro Arg | |
| 625 630 635 | |
| tcc ggg cgg ctc caa gag aga gcg gag caa gtg tcc cgg gcc ctt cag | 2196 |
| Ser Gly Arg Leu Gln Glu Arg Ala Glu Gln Val Ser Arg Ala Leu Gln | |
| 640 645 650 | |
| cca gcc ctg gat agc tac ttc cat ccc ccg ggg acn tcc gcg ccg gga | 2244 |
| Pro Ala Leu Asp Ser Tyr Phe His Pro Pro Gly Xaa Ser Ala Pro Gly | |
| 655 660 665 | |
| cgc ggg gtg gga cca ggg gcg gga cct ggg gcg ggg gac ggg act | 2289 |
| Arg Gly Val Gly Pro Gly Ala Gly Pro Gly Ala Gly Asp Gly Thr | |
| 670 675 680 | |
| taaataaagg cagacgctg | 2308 |

MPVPWFLLSLALGRSQWILSLERLVGPQDATHCSPGLSCRLWDSDILCLPGDIVPAPGPVLAPTHLQTELVL
RCQKETDCDLCLRVAVHLAVHGHWEEPEDEEKFGGAADLGVEEPRNASLQAQVVLSFQAYPTARCVLLEVQV
PAALVQFGQSVGSVVYDCFEAALGSEVRIWSYTQPRYEKELNHTQQLPDCRGLEVWNSIPSCWALPWLNVSA
DGDNVHLVLNVSEEQHFGLSLYWNQVQGPPKPRWHKNLTGPQIITLNHTDLVPCLCIQVWPLEPDSVRTNTC
PFREDPRAHQNLWQAARLRLLTLQSWLLDAPCSLPAEAALCWRAPGGDPCQPLVPPLSWENVTVDVNSSEKL
QLQECLWADSLGPLKDDVLLLETRGPQDNRSLCALEPSGCTSLPSKASTRAARLGEYLLQDLQSGQCLQLWD
DDLGALWACPMDKYIHKRWALVWLACLLFAAALSLILLLKKDHAKGWLRLLKQDVRSGAAARGRAALLLYSA
DDSGFERLVGALASALCQLPLRVAVDLWSRRELSAQGPVAWFHAQRRQTLQEGGVVVLLFSPGAVALCSEWL
QDGVSGPGAHGPHDAFRASLSCVLPDFLQGRAPGSYVGACFDRLLHPDAVPALFRTVPVFTLPSQLPDFLGA
LQQPRAPRSGRLQERAEQVSRALQPALDSYFHPPGTSAPGRGVGPGAGPGAGDGT.

| Reverse translation of primate, e.g., human, DCRS7: (SEQ ID NO:9) | |
|---|---|
| atgccngtnc cntggttyyt nytnwsnytn gcnytnggnm gnwsncartg gathytnwsn | 60 |
| ytngarmgny tngtnggncc ncargaygcn acncaytgyw snccnggnyt nwsntgymgn | 120 |
| ytntgggayw sngayathyt ntgyytnccn ggngayathg tnccngcncc nggnccngtn | 180 |
| ytngcnccna cncayytnca racngarytn gtnytnmgnt gycaraarga racngaytgy | 240 |
| gayytntgyy tnmgngtngc ngtncayytn gcngtncayg gncaytggga rgarccngar | 300 |
| gaygargara arttyggngg ngcngcngay ytnggngtng argarccnmg naaygcnwsn | 360 |
| ytncargcnc argtngtnyt nwsnttycar gcntayccna cngcnmgntg ygtnytnytn | 420 |
| gargtncarg tnccngcngc nytngtncar ttyggncarw sngtnggnws ngtngtntay | 480 |
| gaytgyttyg argcngcnyt nggnwsngar gtnmgnatht ggwsntayac ncarccnmgn | 540 |
| taygaraarg arytnaayca yacncarcar ytnccngayt gymgnggnyt ngargtntgg | 600 |
| aaywsnathc cnwsntgytg ggcnytnccn tggytnaayg tnwsngcnga yggngayaay | 660 |
| gtncayytng tnytnaaygt nwsngargar carcayttyg gnytnwsnyt ntaytggaay | 720 |
| cargtncarg gnccnccnaa rccnmgntgg cayaaraayy tnacnggncc ncarathath | 780 |
| acnytnaayc ayacngayyt ngtnccntgy ytntgyathc argtntggcc nytngarccn | 840 |
| gaywsngtnm gnacnaayat htgyccntty mgngargayc cnmgngcnca ycaraayytn | 900 |
| tggcargcng cnmgnytnmg nytnytnacn ytncarwsnt ggytnytnga ygcnccntgy | 960 |
| wsnytnccng cngargcngc nytntgytgg mgngcnccng gnggngaycc ntgycarccn | 1020 |
| ytngtnccnc cnytnwsntg ggaraaygtn acngtngayg tnaaywsnws ngaraarytn | 1080 |
| carytncarg artgyytntg ggcngaywsn ytnggnccny tnaargayga ygtnytnytn | 1140 |
| ytngaracnm gnggnccnca rgayaaymgn wsnytntgyg cnytngarcc nwsnggntgy | 1200 |
| acnwsnytnc cnwsnaargc nwsnacnmgn gcngcnmgny tnggngarta yytnytncar | 1260 |
| gayytncarw snggncartg yytncarytn tgggaygayg ayytnggngc nytntgggcn | 1320 |
| tgyccnatgg ayaartayat hcayaarmgn tgggcnytng tntggytngc ntgyytnytn | 1380 |
| ttygcngcng cnytnwsnyt nathytnytn ytnaaraarg aycaygcnaa rggntggytn | 1440 |
| mgnytnytna arcargaygt nmgnwsnggn gcngcngcnm gnggnmgngc ngcnytnytn | 1500 |
| ytntaywsng cngaygayws nggnttygar mgnytngtng gngcnytngc nwsngcnytn | 1560 |
| tgycarytnc cnytnmgngt ngcngtngay ytntggwsnm gnmgngaryt nwsngcncar | 1620 |
| ggnccngtng cntggttyca ygcncarmgn mgncaracny tncargargg nggngtngtn | 1680 |
| gtnytnytnt tywsnccngg ngcngtngcn ytntgywsng artggytnca rgayggngtn | 1740 |
| wsnggnccng gngcncaygg nccncaygay gcnttymgng cnwsnytnws ntgygtnytn | 1800 |
| ccngayttyy tncarggnmg ngcnccnggn wsntaygtng gngcntgytt ygaymgnytn | 1860 |
| ytncayccng aygcngtncc ngcnytntty mgnadngtnc cngtnttyac nytnccnwsn | 1920 |
| carytnccng ayttyytngg ngcnytncar carccnmgng cnccnmgnws nggnmgnytn | 1980 |
| cargarmgng cngarcargt nwsnmgngcn ytncarccng cnytngayws ntayttycay | 2040 |
| ccnccnggna cnwsngcncc nggnmgnggn gtnggnccng gngcnggncc nggngcnggn | 2100 |
| gayggnacn | 2109 |

| Rodent, e.g., mouse, embodiment (see SEQ ID NO:10 and 11). | |
|---|---|
| Predicted signal sequence indicated, but may vary by a few | |
| positions and depending upon cell type. | |
| ccaaatcgaa agcacgggag ctgatactgg gcctggagtc caggctcact ggagtgggga | 60 |
| agcatggctg gagaggaatt ctagcccttg ctctctccca gggacacggg gctgattgtc | 120 |
| agcaggggcg aggggtctgc ccccccttgg gggggcagga cggggcctca ggcctgggtg | 180 |
| ctgtccggca cctggaag atg cct gtg tcc tgg ttc ctg ctg tcc ttg gca | 231 |
| Met Pro Val Ser Trp Phe Leu Leu Ser Leu Ala | |
| −20 −15 10 | |
| ctg ggc cga aac cct gtg gtc gtc tct ctg gag aga ctg atg gag cct | 279 |
| Leu Gly Arg Asn Pro Val Val Val Ser Leu Glu Arg Leu Met Glu Pro | |
| −5 −1 1 5 | |
| cag gac act gca cgc tgc tct cta ggc ctc tcc tgc cac ctc tgg gat | 327 |
| Gln Asp Thr Ala Arg Cys Ser Leu Gly Leu Ser Cys His Leu Trp Asp | |
| 10 15 20 | |
| ggt gac gtg ctc tgc ctg cct gga agc ctc cag tct gcc cca ggc cct | 375 |
| Gly Asp Val Leu Cys Leu Pro Gly Ser Leu Gln Ser Ala Pro Gly Pro | |
| 25 30 35 | |
| gtg cta gtg cct acc cgc ctg cag acg gag ctg gtg ctg agg tgt cca | 423 |
| Val Leu Val Pro Thr Arg Leu Gln Thr Glu Leu Val Leu Arg Cys Pro | |
| 40 45 50 55 | |
| cag aag aca gat tgc gcc ctc tgt gtc cgt gtg gtg gtc cac ttg gcc | 471 |
| Gln Lys Thr Asp Cys Ala Leu Cys Val Arg Val Val Val His Leu Ala | |
| 60 65 70 | |
| gtg cat ggg cac tgg gca gag cct gaa gaa gct gga aag tct gat tca | 519 |
| Val His Gly His Trp Ala Glu Pro Glu Glu Ala Gly Lys Ser Asp Ser | |
| 75 80 85 | |
| gaa ctc cag gag tct agg aac gcc tct ctc cag gcc cag gtg gtg ctc | 567 |
| Glu Leu Gln Glu Ser Arg Asn Ala Ser Leu Gln Ala Gln Val Val Leu | |
| 90 95 100 | |
| tcc ttc cag gcc tac ccc atc gcc cgc tgt gcc ctg ctg gag gtc cag | 615 |
| Ser Phe Gln Ala Tyr Pro Ile Ala Arg Cys Ala Leu Leu Glu Val Gln | |
| 105 110 115 | |
| gtg ccc gct gac ctg gtg cag cct ggt cag tcc gtg ggt tct gcg gta | 663 |
| Val Pro Ala Asp Leu Val Gln Pro Gly Gln Ser Val Gly Ser Ala Val | |
| 120 125 130 135 | |
| ttt gac tgt tte gag gct agt ctt ggg gct gag gta cag atc tgg tcc | 711 |
| Phe Asp Cys Phe Glu Ala Ser Leu Gly Ala Glu Val Gln Ile Trp Ser | |
| 140 145 150 | |
| tac acg aag ccc agg tac cag aaa gag ctc aac ctc aca cag cag ctg | 759 |
| Tyr Thr Lys Pro Arg Tyr Gln Lys Glu Leu Asn Leu Thr Gln Gln Leu | |
| 155 160 165 | |
| cct gac tgc agg ggt ctt gaa gtc cgg gac agc atc cag agc tgc tgg | 807 |
| Pro Asp Cys Arg Gly Leu Glu Val Arg Asp Ser Ile Gln Ser Cys Trp | |
| 170 175 180 | |
| gte ctg ccc tgg ctc aat gtg tct aca gat ggt gac aat gtc ctt ctg | 855 |
| Val Leu Pro Trp Leu Asn Val Ser Thr Asp Gly Asp Asn Val Leu Leu | |
| 185 190 195 | |
| aca ctg gat gtc tct gag gag cag gac ttt agc ttc tta ctg tac ctg | 903 |
| Thr Leu Asp Val Ser Glu Glu Gln Asp Phe Ser Phe Leu Leu Tyr Leu | |
| 200 205 210 215 | |
| cgt cca gtc ccg gat gct ctc aaa tcc ttg tgg tac aaa aac ctg act | 951 |
| Arg Pro Val Pro Asp Ala Leu Lys Ser Leu Trp Tyr Lys Asn Leu Thr | |
| 220 225 230 | |
| gga cct cag aac att act tta aac cac aca gac ctg gtt ccc tgc etc | 999 |
| Gly Pro Gln Asn Ile Thr Leu Asn His Thr Asp Leu Val Pro Cys Leu | |
| 235 240 245 | |
| tgc att cag gtg tgg tcg cta gag cca gac tct gag agg gtc gaa ttc | 1047 |
| Cys Ile Gln Val Trp Ser Leu Glu Pro Asp Ser Glu Arg Val Glu Phe | |
| 250 255 260 | |
| tgc ccc ttc cgg gaa gat ccc ggt gca cac agg aac ctc tgg cac ata | 1095 |
| Cys Pro Phe Arg Glu Asp Pro Gly Ala His Arg Asn Leu Trp His Ile | |
| 265 270 275 | |
| gcc agg ctg cgg gta ctg tce cca ggg gta tgg cag cta gat gcg cct | 1143 |
| Ala Arg Leu Arg Val Leu Ser Pro Gly Val Trp Gln Leu Asp Ala Pro | |
| 280 285 290 295 | |
| tgc tgt ctg ccg ggc aag gta aca ctg tgc tgg cag gca cca gac cag | 1191 |
| Cys Cys Leu Pro Gly Lys Val Thr Leu Cys Trp Gln Ala Pro Asp Gln | |
| 300 305 310 | |
| agt ccc tgc cag cca ctt gtg cca cca gtg ccc cag aag aac gcc act | 1239 |
| Ser Pro Cys Gln Pro Leu Val Pro Pro Val Pro Gln Lys Asn Ala Thr | |
| 315 320 325 | |
| gtg aat gag cca caa gat ttc cag ttg gtg gca ggc cac ccc aac ctc | 1287 |
| Val Asn Glu Pro Gln Asp Phe Gln Leu Val Ala Gly His Pro Asn Leu | |
| 330 335 340 | |
| tgt gtc cag gtg agc acc tgg gag aag gtt cag ctg caa gcg tgc ttg | 1335 |
| Cys Val Gln Val Ser Thr Trp Glu Lys Val Gln Leu Gln Ala Cys Leu | |
| 345 350 355 | |
| tgg gct gac tcc ttg ggg ccc ttc aag gat gat atg ctg tta gtg gag | 1383 |
| Trp Ala Asp Ser Leu Gly Pro Phe Lys Asp Asp Met Leu Leu Val Glu | |
| 360 365 370 375 | |
| atg aaa acc ggc ctc aac aac aca tca gte tgt gcc ttg gaa ccc agt | 1431 |
| Met Lys Thr Gly Leu Asn Asn Thr Ser Val Cys Ala Leu Glu Pro Ser | |
| 380 385 390 | |
| ggc tgt aca cca ctg ccc agc atg gcc tcc acg aga gct gct cgc ctg | 1479 |
| Gly Cys Thr Pro Leu Pro Ser Met Ala Ser Thr Arg Ala Ala Arg Leu | |
| 395 400 405 | |
| gga gag gag ttg ctg caa gac ttc cga tca cac cag tgt atg cag ctg | 1527 |
| Gly Glu Glu Leu Leu Gln Asp Phe Arg Ser His Gln Cys Met Gln Leu | |
| 410 415 420 | |
| tgg aac gat gac aac atg gga tcg cta tgg gcc tgc ccc atg gac aag | 1575 |
| Trp Asn Asp Asp Asn Met Gly Ser Leu Trp Ala Cys Pro Met Asp Lys | |
| 425 430 435 | |
| tac atc cac agg cgc tgg gtc cta gta tgg ctg gcc tgc cta ctc ttg | 1623 |
| Tyr Ile His Arg Arg Trp Val Leu Val Trp Leu Ala Cys Leu Leu Leu | |
| 440 445 450 455 | |
| gct gcg gcg ctt ttc ttc ttc ctc ctt cta aaa aag gac cgc agg aaa | 1671 |
| Ala Ala Ala Leu Phe Phe Phe Leu Leu Leu Lys Lys Asp Arg Arg Lys | |
| 460 465 470 | |
| gcg gcc cgt ggc tcc cgc acg gcc ttg ctc ctc cac tcc gcc gac gga | 1719 |
| Ala Ala Arg Gly Ser Arg Thr Ala Leu Leu Leu His Ser Ala Asp Gly | |
| 475 480 485 | |
| gcg ggc tac gag cgc ctg gtg gga gca ctg gcg tcc gcg ttg agc cag | 1767 |
| Ala Gly Tyr Glu Arg Leu Val Gly Ala Leu Ala Ser Ala Leu Ser Gln | |
| 490 495 500 | |
| atg cca ctg cgc gtg gcc gtg gac ctg tgg agc cgc cgc gag ctg agc | 1815 |
| Met Pro Leu Arg Val Ala Val Asp Leu Trp Ser Arg Arg Glu Leu Ser | |
| 505 510 515 | |
| gcg cac gga gcc cta gcc tgg ttc cac cac cag cga cgc cgt atc ctg | 1863 |
| Ala His Gly Ala Leu Ala Trp Phe His His Gln Arg Arg Arg Ile Leu | |
| 520 525 530 535 | |
| cag gag ggt ggc gtg gta atc ctt ctc ttc tcg ccc gcg gcc gtg gcg | 1911 |
| Gln Glu Gly Gly Val Val Ile Leu Leu Phe Ser Pro Ala Ala Val Ala | |
| 540 545 550 | |
| cag tgt cag cag tgg ctg cag ctc cag aca gtg gag ccc ggg ccg cat | 1959 |
| Gln Cys Gln Gln Trp Leu Gln Leu Gln Thr Val Glu Pro Gly Pro His | |
| 555 560 565 | |
| gac gcc ctc gcc gcc tgg ctc agc tgc gtg cta ccc gat ttc ctg caa | 2007 |
| Asp Ala Leu Ala Ala Trp Leu Ser Cys Val Leu Pro Asp Phe Leu Gln | |
| 570 575 580 | |
| ggc cgg gcg acc ggc cgc tac gtc ggg gtc tac ttc gac ggg ctg ctg | 2055 |
| Gly Arg Ala Thr Gly Arg Tyr Val Gly Val Tyr Phe Asp Gly Leu Leu | |
| 585 590 595 | |
| cac cca gac tct gtg ccc tcc ccg ttc cgc gtc gcc ccg ctc ttc tcc | 2103 |
| His Pro Asp Ser Val Pro Ser Pro Phe Arg Val Ala Pro Leu Phe Ser | |
| 600 605 610 615 | |
| ctg ccc tcg cag ctg ccg gct ttc ctg gat gca ctg cag gga ggc tgc | 2151 |
| Leu Pro Ser Gln Leu Pro Ala Phe Leu Asp Ala Leu Gln Gly Gly Cys | |
| 620 625 630 | |
| tcc act tcc gcg ggg cga ccc gcg gac cgg gtg gaa cga gtg acc cag | 2199 |
| Ser Thr Ser Ala Gly Arg Pro Ala Asp Arg Val Glu Arg Val Thr Gln | |
| 635 640 645 | |
| gcg ctg cgg tcc gcc ctg gac agc tgt act tct agc tcg gaa gcc cca | 2247 |
| Ala Leu Arg Ser Ala Len Asp Ser Cys Thr Ser Ser Ser Glu Ala Pro | |
| 650 655 660 | |
| ggc tgc tgc gag gaa tgg gac ctg gga ccc tgc act aca cta gaa | 2292 |
| Gly Cys Cys Gln Gln Trp Asp Leu Gly Pro Cys Thr Thr Leu Gln | |
| 665 670 675 | |
| taaaagccga tacagtattc ct | 2314 |

MPVSWFLLSLALGRNPVVVSLERLMEPQDTARCSLGLSCHLWDGDVLCLPGSLQSAPGPVLVPTRLQTELVL
RCPQKTDCALCVRVVVHLAVHGHWAEPEEAGKSDSELQESRNASLQAQVVLSFQAYPIARCALLEVQVPADL
VQPGQSVGSAVFDCFEASLGAEVQIWSYTKPRYQKELNLTQQLPDCRGLEVRDSIQSCWVLPWLNVSTDGDN
VLLTLDVSEEQDFSFLLYLRPVPDALKSLWYKNLTGPQNITLNHTDLVPCLCIQVWSLEPDSERVEFCPFRE
DPGAHRNLWHIARLRVLSPGVWQLDAPCCLPGKVTLCWQAPDQSPCQPLVPPVPQKNATVNEPQDFQLVAGH
PNLCVQVSTWEKVQLQACLWADSLGPFKDDMLLVEMKTGLNNTSVCALEPSGCTPLPSMASTRAARLGEELL
QDFRSHQCMQLWNDDNMGSLWACPMDKYIHRRWVLVWLACLLLAAALFFFLLLKKDRRKAARGSRTALLLHS
ADGAGYERLVGALASALSQMPLRVAVDLWSRRELSAHGALAWFHHQRRRILQEGGVVILLFSPAAVAQCQQW
LQLQTVEPGPHDALAAWLSCVLPDFLQGRATGRYVGVYFDGLLHPDSVPSPFRVAPLFSLPSQLPAFLDALQ
GGCSTSAGRPADRVERVTQALRSALDSCTSSSEAPGCCEEWDLGPCTTLE.

| Reverse translation of rodent e.g., mouse, DCRS7 (SEQ ID NO:12): | |
|---|---|
| atgccngtnw sntggttyyt nytnwsnytn gcnytnggnm gnaayccngt ngtngtnwsn | 60 |
| ytngarmgny tnatggarcc ncargayacn gcnmgntgyw snytnggnyt nwsntgycay | 120 |
| ytntgggayg gngaygtnyt ntgyytnccn ggnwsnytnc arwsngcncc nggnccngtn | 180 |
| ytngtnccna cnmgnytnca racngarytn gtnytnmgnt gyccncaraa racngaytgy | 240 |
| gcnytntgyg tnmgngtngt ngtncayytn gcngtncayg gncaytgggc ngarccngar | 300 |
| gargcnggna arwsngayws ngarytncar garwsnmgna aygcnwsnyt ncargcncar | 360 |
| gtngtnytnw snttycargc ntayccnath gcnmgntgyg cnytnytnga rgtncargtn | 420 |
| ccngcngayy tngtncarcc nggncarwsn gtnggnwsng cngtnttyga ytgyttygar | 480 |
| gcnwsnytng gngcngargt ncarathtgg wsntayacna arccnmgnta ycaraargar | 540 |
| ytnaayytna cncarcaryt nccngaytgy mgnggnytng argtnmgnga ywsnathcar | 600 |
| wsntgytggg tnytnccntg gytnaaygtn wsnacngayg gngayaaygt nytnytnacn | 660 |
| ytngaygtnw sngargarca rgayttywsn ttyytnytnt ayytnmgncc ngtnccngay | 720 |
| gcnytnaarw snytntggta yaaraayytn acnggnccnc araayathac nytnaaycay | 780 |
| acngayytng tnccntgyyt ntgyathcar gtntggwsny tngarccnga ywsngarmgn | 840 |
| gtngarttyt gyccnttymg ngargayccn ggngcncaym gnaayytntg gcayathgcn | 900 |
| mgnytnmgng tnytnwsncc nggngtntgg carytngayg cnccntgytg yytnccnggn | 960 |
| aargtnacny tntgytggca rgcnccngay carwsnccnt gycarccnyt ngtnccnccn | 1020 |
| gtnccncara araaygcnac ngtnaaygar ccncargayt tycarytngt ngcnggncay | 1080 |
| ccnaayytnt gygtncargt nwsnacntgg garaargtnc arytncargc ntgyytntgg | 1140 |
| gcngaywsny tnggnccntt yaargaygay atgytnytng tngaratgaa racnggnytn | 1200 |
| aayaayacnw sngtntgygc nytngarccn wsnggntgya cnccnytncc nwsnatggcn | 1260 |
| wsnacnmgng cngcnmgnyt nggngargar ytnytncarg ayttymgnws ncaycartgy | 1320 |
| atgcarytnt ggaaygayga yaayatgggn wsnytntggg cntgyccnat ggayaartay | 1380 |
| athcaymgnm gntgggtnyt ngtntggytn gcntgyytny tnytngcngc ngcnytntty | 1440 |
| ttyttyytny tnytnaaraa rgaymgnmgn aargcngcnm gnggnwsnmg nacngcnytn | 1500 |
| ytnytncayw sngcngaygg ngcnggntay garmgnytng tnggngcnyt ngcnwsngcn | 1560 |
| ytnwsncara tgccnytnmg ngtngcngtn gayytntggw snmgnmgnga rytnwsngcn | 1620 |
| cayggngcny tngcntggtt ycaycaycar mgnmgnmgna thytncarga rggnggngtn | 1680 |
| gtnathytny tnttywsncc ngcngcngtn gcncartgyc arcartggyt ncarytncar | 1740 |
| acngtngarc cnggnccnca ygaygcnytn gcngcntggy tnwsntgygt nytnccngay | 1800 |
| ttyytncarg gnmgngcnac nggnmgntay gtnggngtnt ayttygaygg nytnytncay | 1860 |
| ccngaywsng tnccnwsncc nttymgngtn gcnccnytnt tywsnytncc nwsncarytn | 1920 |
| ccngcnttyy tngaygcnyt ncarggnggn tgywsnacnw sngcnggnmg nccngcngay | 1980 |
| mgngtngarm gngtnacnca rgcnytnmgn wsngcnytng aywsntgyac nwsnwsnwsn | 2040 |
| gargcnccng gntgytgyga rgartgggay ytnggnccnt gyacnacnyt ngar | 2094 |

TABLE 3
Nucleotide and polypeptide sequences of DNAX Cytokine Receptor Subunit
like embodiments (DCRS8). Primate, e.g., human, embodiment
(see SEQ ID NO:13 and 14). Predicted signal sequence indicated, but may
vary by a few positions and depending upon cell type.

| | |
|---|---|
| cccacgcntc cgggccagca gcgggcggcc ggggcgcaga gaacggcctg gctgggcgag | 60 |
| cgcacggcc atg gcc ccg tgg ctg cag ctc tgc tcc gtc ttc ttt acg gtc | 111 |
| Met Ala Pro Trp Leu Gln Leu Cys Ser Val Phe Phe Thr Val | |
| −15 −10 −5 | |
| aac gcc tgc ctc aac ggc tcg cag ctg gct gtn gcc gct ggc ggg tcc | 159 |
| Asn Ala Cys Leu Asn Gly Ser Gln Leu Ala Xaa Ala Ala Gly Gly Ser | |
| −1 5 10 | |
| ggc cgc gcg cng ggc gcc gac acc tgt agc tgg ang gga gtg ggg cca | 207 |
| Gly Arg Ala Xaa Gly Ala Asp Thr Cys Ser Trp Xaa Gly Val Gly Pro | |
| 15 20 25 30 | |
| gcc agc aga aac agt ggg ctg tac aac atc acc ttc aaa tat gac aat | 255 |
| Ala Ser Arg Asn Ser Gly Leu Tyr Asn Ile Thr Phe Lys Tyr Asp Asn | |
| 35 40 45 | |
| tgt acc acc tac ttg aat cca gtg ggg aag cat gtg att gct gac gcc | 303 |
| Cys Thr Thr Tyr Leu Asn Pro Val Gly Lys His Val Tie Ala Asp Ala | |
| 50 55 60 | |
| cag aat atc acc atc agc cag tat gct tgc cat gac caa gtg gca gtc | 351 |
| Gln Asn Ile Thr Ile Ser Gln Tyr Ala Cys His Asp Gln Val Ala Val | |
| 65 70 75 | |
| acc att ctt tgg tcc cca ggg gcc ctc ggc atc gaa ttc ctg aaa gga | 399 |
| Thr Ile Leu Trp Ser Pro Gly Ala Leu Gly Ile Glu Phe Leu Lys Gly | |
| 80 85 90 | |
| ttt cgg gta ata ctg gag gag ctg aag tcg gag gga aga cag ngc caa | 447 |
| Phe Arg Val Ile Leu Glu Glu Leu Lys Ser Glu Gly Arg Gln Xaa Gln | |
| 95 100 105 110 | |
| caa ctg att cta aag gat ccg aag cag ntc aac agt agc ttc aaa aga | 495 |
| Gln Leu Ile Leu Lys Asp Pro Lys Gln Xaa Asn Ser Ser Phe Lys Arg | |
| 115 120 125 | |
| act gga atg gaa tct caa cct ttn ctg aat atg aaa ttt gaa acg gat | 543 |
| Thr Gly Met Glu Ser Gln Pro Xaa Leu Asn Met Lys Phe Glu Thr Asp | |
| 130 135 140 | |
| tat ttc gta agg ttg tcc ttt tcc ttc att aaa aac gaa agc aat tac | 591 |
| Tyr Phe Val Arg Leu Ser Phe Ser Phe Ile Lys Asn Glu Ser Asn Tyr | |
| 145 150 155 | |
| cac cct ttc ttc ttt aga acc cga gcc tgt gac ctg ttg tta cag ccg | 639 |
| His Pro Phe Phe Phe Arg Thr Arg Ala Cys Asp Leu Leu Leu Gln Pro | |
| 160 165 170 | |
| gac aat cta gct tgt aaa ccc ttc tgg aag cct cgg aac ctg aac atc | 687 |
| Asp Asn Leu Ala Cys Lys Pro Phe Trp Lys Pro Arg Asn Leu Asn Ile | |
| 175 180 185 190 | |
| agc cag cat ggc tcg gac atg cag gtg tcc ttc gac cac gca ccg cac | 735 |
| Ser Gln His Gly Ser Asp Met Gln Val Ser Phe Asp His Ala Pro His | |
| 195 200 205 | |
| aac ttc ggc ttc cgt ttc ttc tat ctt cac tac aag ctc aag cac gaa | 783 |
| Asn Phe Gly Phe Arg Phe Phe Tyr Leu His Tyr Lys Leu Lys His Glu | |
| 210 215 220 | |
| gga cct ttc aag cga aag acc tgt aag cag gag caa act aca gag atg | 831 |
| Gly Pro Phe Lys Arg Lys Thr Cys Lys Gln Glu Gln Thr Thr Glu Met | |
| 225 230 235 | |
| acc agc tgc ctc ctt caa aat gtt tct cca ggg gat tat ata att gag | 879 |
| Thr Ser Cys Leu Leu Gln Asn Val Ser Pro Gly Asp Tyr Ile Ile Glu | |
| 240 245 250 | |
| ctg gtg gat gac act aac aca aca aga aaa gtg atg cat tat gcc tta | 927 |
| Leu Val Asp Asp Thr Asn Thr Thr Arg Lys Val Met His Tyr Ala Leu | |
| 255 260 265 270 | |
| aag cca gtg cac tcc ccg tgg gcc ggg ccc atc aga gcc gtg gcc atc | 975 |
| Lys Pro Val His Ser Pro Trp Ala Gly Pro Ile Arg Ala Val Ala Ile | |
| 275 280 285 | |
| aca gtg cca ctg gta gtc ata tcg gca ttc gcg acg ctc ttc act gtg | 1023 |
| Thr Val Pro Leu Val Val Ile Ser Ala Phe Ala Thr Leu Phe Thr Val | |
| 290 295 300 | |
| atg tgc cgc aag aag caa caa gaa aat ata tat tca cat tta gat gaa | 1071 |
| Met Cys Arg Lys Lys Gln Gln Glu Asn Ile Tyr Ser His Leu Asp Glu | |
| 305 310 315 | |
| gag agc tct gag tct tcc aca tac act gca gca ctc cca aga gag agg | 1119 |
| Glu Ser Ser Glu Ser Ser Thr Tyr Thr Ala Ala Leu Pro Arg Glu Arg | |
| 320 325 330 | |
| ctc cgg ccg cgg ccg aag gtc ttt ctc tgc tat tcc agt aaa gat ggc | 1167 |
| Leu Arg Pro Arg Pro Lys Val Phe Leu Cys Tyr Ser Ser Lys Asp Gly | |
| 335 340 345 350 | |
| cag aat cac atg aat gtc gtc cag tgt ttc gcc tac ttc ctc cag gac | 1215 |
| Gln Asn His Met Asn Val Val Gln Cys Phe Ala Tyr Phe Leu Gln Asp | |
| 355 360 365 | |
| ttc tgt ggc tgt gag gtg gct ctg gac ctg tgg gaa gac ttc agc ctc | 1263 |
| Phe Cys Gly Cys Glu Val Ala Leu Asp Leu Trp Glu Asp Phe Ser Leu | |
| 370 375 380 | |
| tgt aga gaa ggg cag aga gaa tgg gtc atc cag aag atc cac gag tcc | 1311 |
| Cys Arg Glu Gly Gln Arg Glu Trp Val Ile Gln Lys Ile His Glu Ser | |
| 385 390 395 | |
| cag ttc atc att gtg gtt tgt tcc aaa ggt atg aag tac ttt gtg gac | 1359 |
| Gln Phe Ile Ile Val Val Cys Ser Lys Gly Met Lys Tyr Phe Val Asp | |
| 400 405 410 | |
| aag aag aac tac aaa cac aaa gga ggt ggc cga ggc tcg ggg aaa gga | 1407 |
| Lys Lys Asn Tyr Lys His Lys Gly Gly Gly Arg Gly Ser Gly Lys Gly | |
| 415 420 425 430 | |
| gag ctc ttc ctg gtg gcg gtg tca gcc att gcc gaa aag ctc cgc cag | 1455 |
| Glu Leu Phe Leu Val Ala Val Ser Ala Ile Ala Glu Lys Leu Arg Gln | |
| 435 440 445 | |
| gcc aag cag agt tcg tcc gcg gcg ctc agc aag ttt atc gcc gtc tac | 1503 |
| Ala Lys Gln Ser Ser Ser Ala Ala Leu Ser Lys Phe Ile Ala Val Tyr | |
| 450 455 460 | |
| ttt gat tat tcc tgc gag gga gac gtc ccc ggt atc cta gac ctg agt | 1551 |
| Phe Asp Tyr Ser Cys Glu Gly Asp Val Pro Gly Ile Leu Asp Leu Ser | |
| 465 470 475 | |
| acc aag tac aga ctc atg gac aat ctt cct cag ctc tgt tcc cac ctg | 1599 |
| Thr Lys Tyr Arg Leu Met Asp Asn Leu Pro Gln Leu Cys Ser His Leu | |
| 480 485 490 | |
| cac tcc cga gac cac ggc ctc cag gag ccg ggg cag cac acg cga cag | 1647 |
| His Ser Arg Asp His Gly Leu Gln Glu Pro Gly Gln His Thr Arg Gln | |
| 495 500 505 510 | |
| ggc agc aga agg aac tac ttc cgg agc aag tca ggc cgg tcc cta tac | 1695 |
| Gly Ser Arg Arg Asn Tyr Phe Arg Ser Lys Ser Gly Arg Ser Leu Tyr | |
| 515 520 525 | |
| gtc gcc att tgc aac atg cac cag ttt att gac gag gag ccc gac tgg | 1743 |
| Val Ala Ile Cys Asn Met His Gln Phe Ile Asp Glu Glu Pro Asp Trp | |
| 530 535 540 | |
| ttc gaa aag cag ttc gtt ccc ttc cat cct cct cca ctg cgc tac cgg | 1791 |
| Phe Glu Lys Gln Phe Val Pro Phe His Pro Pro Pro Leu Arg Tyr Arg | |
| 545 550 555 | |
| gag cca gtc ttg gag aaa ttt gat tcg ggc ttg gtt tta aat gat gtc | 1839 |
| Glu Pro Val Leu Glu Lys Phe Asp Ser Gly Leu Val Leu Asn Asp Val | |
| 560 565 570 | |
| atg tgc aaa cca ggg cct gag agt gac tte tgc cta aag gta gag gcg | 1887 |
| Met Cys Lys Pro Gly Pro Glu Ser Asp Phe Cys Leu Lys Val Glu Ala | |
| 575 580 585 590 | |
| gct gtt ctt ggg gca acc gga cca gcc gac tcc cag cac gag agt cag | 1935 |
| Ala Val Leu Gly Ala Thr Gly Pro Ala Asp Ser Gln His Glu Ser Gln | |
| 595 600 605 | |
| cat ggg ggc ctg gac caa gac ggg gag gcc cgg cct gcc ctt gac ggt | 1983 |
| His Gly Gly Leu Asp Gln Asp Gly Glu Ala Arg Pro Ala Leu Asp Gly | |
| 610 615 620 | |
| agc gcc gcc ctg caa ccc ctg ctg cac acg gtg aaa gcc ggc agc ccc | 2031 |
| Ser Ala Ala Leu Gln Pro Leu Leu His Thr Val Lys Ala Gly Ser Pro | |
| 625 630 635 | |
| tcg gac atg ccg cgg gac tca ggc atc tat gac tcg tct gtg ccc tca | 2079 |
| Ser Asp Met Pro Arg Asp Ser Gly Ile Tyr Asp Ser Ser Val Pro Ser | |
| 640 645 650 | |
| tcc gag ctg tct ctg cca ctg atg gaa gga ctc tcg acg gac cag aca | 2127 |
| Ser Glu Leu Ser Leu Pro Leu Met Glu Gly Leu Ser Thr Asp Gln Thr | |
| 655 660 665 670 | |
| gaa acg tct tcc ctg acg gag agc gtg tcc tcc tct tca ggc ctg ggt | 2175 |
| Glu Thr Ser Ser Leu Thr Glu Ser Val Ser Ser Ser Ser Gly Leu Gly | |
| 675 680 685 | |
| gag gag gaa cct cct gcc ctt cct tcc aag ctc ctc tct tct ggg tca | 2223 |
| Glu Glu Glu Pro Pro Ala Leu Pro Ser Lys Leu Leu Ser Ser Gly Ser | |
| 690 695 700 | |
| tgc aaa gca gat ctt ggt tgc cgc agc tac act gat gaa ctc cac gcg | 2271 |
| Cys Lys Ala Asp Leu Gly Cys Arg Ser Tyr Thr Asp Glu Leu His Ala | |
| 705 710 715 | |
| gtc gcc cct ttg taacaaaacg aaagagtcta agcattgcca ctttagctgc | 2323 |
| Val Ala Pro Leu | |
| 720 | |
| tgcctccctc tgattcccca gctcatctcc ctggttgcat ggcccacttg gagctgaggt | 2383 |
| ctcatacaag gatatttgga gtgaaatgct ggccagtact tgttctccct tgccccaacc | 2443 |
| ctttaccgga tatcttgaca aactctccaa ttttctaaaa tgatatggag ctctgaaagg | 2503 |
| catgtccata aggtctgaca acagcttgcc aaatttggtt agtccttgga tcagagcctg | 2563 |
| ttgtgggagg tagggaggaa atatgtaaag aaaaacagga agatacctgc actaatcatt | 2623 |
| cagacttcat tgagctctgc aaactttgcc tgtttgctat tggctacctt gatttgaaat | 2683 |
| gctttgtgaa aaaaggcact tttaacatca tagccacaga aatcaagtgc cagtctatct | 2743 |
| ggaatccatg ttgtattgca gataatgttc tcatttattt ttg | 2786 |

MAPWLQLCSVFFTVNACLNGSQLAVAAGGSGRAXGADTCSWXGVGPASRNSGLYNITFKYDNCTTYLNPVGK
HVIADAQNITISQYACHDQVAVTILWSPGALGIEFLKGFRVILEELKSEGRQXQQLILKDPKQXNSSFKRTG
MESQPXLNMKFETDYFVRLSFSFIKNESNYHPFFFRTRACDLLLQPDNLACKPFWKPRNLNTSQHGSDMQVS
FDHAPHNFGFRFFYHYKLKHEGPFKRKTCKQEQTTEMTSCLLQNVSPGDYIIELVDDTNTTRKVMHYALKP
VHSPWAGPIRAVAITVPLVVISAFATLFTVMCRKKQQENIYSHLDEESSESSTYTAALPRERLRPRPKVFLC
YSSKDGQNHMNVVQCFAYFLQDFCGCEVALDLWEDFSLCREGQREWVIQKIHESQFIIVVCSKGMKYFVDKK
NYKHKGGGRGSGKGELFLVAVSAIAEKLRQAKQSSSAALSKFIAVYFDYSCEGDVPGILDLSTKYRLMDNLP
QLCSHLHSRDHGLQEPGQHTRQGSRRNYFRSKSGRSLYVAICNMHQFIDEEPDWFEKQFVPFHPPPLRYREP
VLEKFDSGLVLNDVMCKPGPESDFCLKVEAAVLGATGPADSQHESQHGGLDQDGEARPALDGSAALQPLLHT
VKAGSPSDMPRDSGIYDSSVPSSELSLPLMEGLSTDQTETSSLTESVSSSSGLGEEEPPALPSKLLSSGSCK
ADLGCRSYTDELHAVAPL.

| Reverse translation of primate, e.g., human, DCRS8: (SEQ ID NO:15) | |
|---|---|
| atggcnccnt ggytncaryt ntgywsngtn ttyttyacng tnaaygcntg yytnaayggn | 60 |
| wsncarytng cngtngcngc nggnggnwsn ggnmgngcnn nnggngcnga yacntgywsn | 120 |
| tggnnnggng tnggnccngc nwsnmgnaay wsnggnytnt ayaayathac nttyaartay | 180 |
| gayaaytgya cnacntayyt naayccngtn ggnaarcayg tnathgcnga ygcncaraay | 240 |
| athacnathw sncartaygc ntgycaygay cargtngcng tnacnathyt ntggwsnccn | 300 |
| ggngcnytng gnathgartt yytnaarggn ttymgngtna thytngarga rytnaarwsn | 360 |
| garggnmgnc arnnncarca rytnathytn aargayccna arcarnnnaa ywsnwsntty | 420 |
| aarmgnacng gnatggarws ncarccnnnn ytnaayatga arttygarac ngaytaytty | 480 |
| gtnmgnytnw snttywsntt yathaaraay garwsnaayt aycayccntt yttyttymgn | 540 |
| acnmgngcnt gygayytnyt nytncarccn gayaayytng cntgyaarcc nttytggaar | 600 |
| ccnmgnaayy tnaayathws ncarcayggn wsngayatgc argtnwsntt ygaycaygcn | 660 |
| ccncayaayt tyggnttymg nttyttytay ytncaytaya arytnaarca ygarggnccn | 720 |
| ttyaarmgna aracntgyaa rcargarcar acnacngara tgacnwsntg yytnytncar | 780 |
| aaygtnwsnc cnggngayta yathathgar ytngtngayg ayacnaayac nacnmgnaar | 840 |
| gtnatgcayt aygcnytnaa rccngtncay wsnccntggg cnggnccnat hmgngcngtn | 900 |
| gcnathacng tnccnytngt ngtnathwsn gcnttygcna cnytnttyac ngtnatgtgy | 960 |
| mgnaaraarc arcargaraa yathtaywsn cayytngayg argarwsnws ngarwsnwsn | 1020 |
| acntayacng cngcnytncc nmgngarmgn ytnmgnccnm gnccnaargt nttyytntgy | 1080 |
| taywsnwsna argayggnca raaycayatg aaygtngtnc artgyttygc ntayttyytn | 1140 |
| cargayttyt gyggntgyga rgtngcnytn gayytntggg argayttyws nytntgymgn | 1200 |
| garggncarm gngartgggt nathcaraar athcaygarw sncarttyat hathgtngtn | 1260 |
| tgywsnaarg gnatgaarta yttygtngay aaraaraayt ayaarcayaa rggnggnggn | 1320 |
| mgnggnwsng gnaarggnga rytnttyytn gtngcngtnw sngcnathgc ngaraarytn | 1380 |
| mgncargcna arcarwsnws nwsngcngcn ytnwsnaart tyathgcngt ntayttygay | 1440 |
| taywsntgyg arggngaygt nccnggnath ytngayytnw snacnaarta ymgnytnatg | 1500 |
| gayaayytnc cncarytntg ywsncayytn caywsnmgng aycayggnyt ncargarccn | 1560 |
| ggncarcaya cnmgncargg nwsnmgnmgn aaytayttym gnwsnaarws nggnmgnwsn | 1620 |
| ytntaygtng cnathtgyaa yatgcaycar ttyathgayg argarccnga ytggttygar | 1680 |
| aarcarttyg tnccnttyca yccnccnccn ytnmgntaym gngarccngt nytngaraar | 1740 |
| ttygaywsng gnytngtnyt naaygaygtn atgtgyaarc cnggnccnga rwsngaytty | 1800 |
| tgyytnaarg tngargcngc ngtnytnggn gcnacnggnc cngcngayws ncarcaygar | 1860 |
| wsncarcayg gnggnytnga ycargayggn gargcnmgnc cngcnytnga yggnwsngcn | 1920 |
| gcnytncarc cnytnytnca yacngtnaar gcnggnwsnc cnwsngayat gccnmgngay | 1980 |
| wsnggnatht aygaywsnws ngtnccnwsn wsngarytnw snytnccnyt natggarggn | 2040 |
| ytnwsnacng aycaracnga racnwsnwsn ytnacngarw sngtnwsnws nwsnwsnggn | 2100 |
| ytnggngarg argarccncc ngcnytnccn wsnaarytny tnwsnwsngg nwsntgyaar | 2160 |
| gcngayytng gntgymgnws ntayacngay garytncayg cngtngcncc nytn | 2214 |

TABLE 4
Nucleotide and polypeptide sequences of DNAX Cytokine Receptor Subunit
like embodiments (DCRS9). Primate, e.g., human, embodiment
(see SEQ ID NO:16 and 17). Predicted signal sequence indicated, but may
vary by a few positions and depending upon cell type.

| | |
|---|---|
| atg ggg agc tcc aga ctg gca gcc ctg ctc ctg cct ctc ctc ctc ata | 48 |
| Met Gly Ser Ser Arg Leu Ala Ala Leu Leu Leu Pro Leu Leu Leu Ile | |
| −20 −15 −10 | |
| gtc atc gac ctc tct gac tct gct ggg att ggc ttt cgc cac ctg ccc | 96 |
| Val Ile Asp Leu Ser Asp Ser Ala Gly Ile Gly Phe Arg His Leu Pro | |
| −5 −1 1 5 | |
| cac tgg aac acc cgc tgt cct ctg gcc tcc cac acg gaa gtt ctg cct | 144 |
| His Trp Asn Thr Arg Cys Pro Leu Ala Ser His Thr Glu Val Leu Pro | |
| 10 15 20 25 | |
| ata tcc ctt gcc gca cct ggt ggg ccc tct tct cca caa agc ctt ggt | 192 |
| Ile Ser Leu Ala Ala Pro Gly Gly Pro Ser Ser Pro Gln Ser Leu Gly | |
| 30 35 40 | |
| gtg tgc gag tct ggc act gtt ccc gct gtt tgt gcc agc atc tgc tgt | 240 |
| Val Cys Glu Ser Gly Thr Val Pro Ala Val Cys Ala Ser Ile Cys Cys | |
| 45 50 55 | |
| cag gtg gct cag gtc ttc aac ggg gcc tct tcc acc tcc tgg tgc aga | 288 |
| Gln Val Ala Gln Val Phe Asn Gly Ala Ser Ser Thr Ser Trp Cys Arg | |
| 60 65 70 | |
| aat cca aaa agt ctt cca cat tca agt tct ata gga gac aca aga tgc | 336 |
| Asn Pro Lys Ser Leu Pro His Ser Ser Ser Ile Gly Asp Thr Arg Cys | |
| 75 80 85 | |
| cag cac ctg ctc aga gga agc tgc tgc ctc gtc gtc acc tgt ctg aga | 384 |
| Gln His Leu Leu Arg Gly Ser Cys Cys Leu Val Val Thr Cys Leu Arg | |
| 90 95 100 105 | |
| aga gcc atc aca ttt cca tcc cct ccc cag aca tct ccc aca agg gac | 432 |
| Arg Ala Ile Thr Phe Pro Ser Pro Pro Gln Thr Ser Pro Thr Arg Asp | |
| 110 115 120 | |
| ttc gct cta aaa gga ccc aac ctt cgg atc cag aga cat ggg aaa gtc | 480 |
| Phe Ala Leu Lys Gly Pro Asn Leu Arg Ile Gln Arg His Gly Lys Val | |
| 125 130 135 | |
| ttc cca gat tgg act cac aaa ggc atg gag gtg ggc act ggg tac aac | 528 |
| Phe Pro Asp Trp Thr His Lys Gly Met Glu Val Gly Thr Gly Tyr Asn | |
| 140 145 150 | |
| agg aga tgg gtt cag ctg agt ggt gga ccc gag ttc tcc ttt gat ttg | 576 |
| Arg Arg Trp Val Gln Leu Ser Gly Gly Pro Glu Phe Ser Phe Asp Leu | |
| 155 160 165 | |
| ctg cct gag gcc cgg gct att cgg gtg acc ata tct tca ggc cct gag | 624 |
| Leu Pro Glu Ala Arg Ala Ile Arg Val Thr Ile Ser Ser Gly Pro Glu | |
| 170 175 180 185 | |
| gtc agc gtg cgt ctt tgt cac cag tgg gca ctg gag tgt gaa gag ctg | 672 |
| Val Ser Val Arg Leu Cys His Gln Trp Ala Leu Glu Cys Glu Glu Leu | |
| 190 195 200 | |
| agc agt ccc tat gat gtc cag aaa att gtg tct ggg ggc cac act gta | 720 |
| Ser Ser Pro Tyr Asp Val Gin Lys Ile Val Ser Gly Gly His Thr Val | |
| 205 210 215 | |
| gag ctg cct tat gaa ttc ctt ctg ccc tgt ctg tgc ata gag gca tcc | 768 |
| Glu Leu Pro Tyr Glu Phe Leu Leu Pro Cys Leu Cys Ile Glu Ala Ser | |
| 220 225 230 | |
| tac ctg caa gag gac act gtg agg cgc aaa aaa tgt ccc ttc cag agc | 816 |
| Tyr Leu Gln Glu Asp Thr Val Arg Arg Lys Lys Cys Pro Phe Gln Ser | |
| 235 240 245 | |
| tgg cca gaa gcc tat ggc tcg gac ttc tgg aag tca gtg cac ttc act | 864 |
| Trp Pro Glu Ala Tyr Gly Ser Asp Phe Trp Lys Ser Val His Phe Thr | |
| 250 255 260 265 | |
| gac tac agc cag cac act cag atg gtc atg gcc ctg aca ctc cgc tgc | 912 |
| Asp Tyr Ser Gln His Thr Gln Met Val Met Ala Leu Thr Leu Arg Cys | |
| 270 275 280 | |
| cca ctg aag ctg gaa gct gcc ctc tgc cag agg cac gac tgg cat acc | 960 |
| Pro Leu Lys Leu Glu Ala Ala Leu Cys Gln Arg His Asp Trp His Thr | |
| 285 290 295 | |
| ctt tgc aaa gac ctc ccg aat gcc acg gct cga gag tca gat ggg tgg | 1008 |
| Leu Cys Lys Asp Leu Pro Asn Ala Thr Ala Arg Glu Ser Asp Gly Trp | |
| 300 305 310 | |
| tat gtt ttg gag aag gtg gac ctg cac ccc cag ctc tgc ttc aag gta | 1056 |
| Tyr Val Leu Glu Lys Val Asp Leu His Pro Gln Leu Cys Phe Lys Val | |
| 315 320 325 | |
| caa cca tgg ttc tct ttt gga aac agc agc cat gtt gaa tgc ccc cac | 1104 |
| Gln Pro Trp Phe Ser Phe Gly Asn Ser Ser His Val Glu Cys Pro His | |
| 330 335 340 345 | |
| cag act ggg tct ctc aca tcc tgg aat gta agc atg gat acc caa gcc | 1152 |
| Gln Thr Gly Ser Leu Thr Ser Trp Asn Val Ser Met Asp Thr Gln Ala | |
| 350 355 360 | |
| cag cag ctg att ctt cac ttc tcc tca aga atg cat gcc acc ttc agt | 1200 |
| Gln Gln Leu Ile Leu His Phe Ser Ser Arg Met His Ala Thr Phe Ser | |
| 365 370 375 | |
| gct gcc tgg agc ctc cca ggc ttg ggg cag gac act ttg gtg ccc ccc | 1248 |
| Ala Ala Trp Ser Leu Pro Gly Leu Gly Gln Asp Thr Leu Val Pro Pro | |
| 380 385 390 | |
| gtg tac act gtc agc cag gtg tgg cgg tca gat gtc cag ttt gcc tgg | 1296 |
| Val Tyr Thr Val Ser Gln Val Trp Arg Ser Asp Val Gln Phe Ala Trp | |
| 395 400 405 | |
| aag cac ctc ttg tgt cca gat gtc tct tac aga cac ctg ggg ctc ttg | 1344 |
| Lys His Leu Leu Cys Pro Asp Val Ser Tyr Arg His Leu Gly Leu Leu | |
| 410 415 420 425 | |
| atc ctg gca ctg ctg gcc ctc ctc acc cta ctg ggt gtt gtt ctg gcc | 1392 |
| Ile Leu Ala Leu Leu Ala Leu Leu Thr Leu Leu Gly Val Val Leu Ala | |
| 430 435 440 | |
| ctc acc tgc cgg cgc cca cag tca ggc ccg ggc cca gcg cgg cca gtg | 1440 |
| Leu Thr Cys Arg Arg Pro Gln Ser Gly Pro Gly Pro Ala Arg Pro Val | |
| 445 450 455 | |
| ctc ctc ctg cac gcg gcg gac tcg gag gcg cag cgg cgc ctg gtg gga | 1488 |
| Leu Leu Leu His Ala Ala Asp Ser Glu Ala Gln Arg Arg Leu Val Gly | |
| 460 465 470 | |
| gcg ctg gct gaa ctg cta cgg gca gcg ctg ggc ggc ggg cgc gac gtg | 1536 |
| Ala Leu Ala Glu Leu Leu Arg Ala Ala Leu Gly Gly Gly Arg Asp Val | |
| 475 480 485 | |
| atc gtg gac ctg tgg gag ggg agg cac gtg gcg cgc gtg ggc ccg ctg | 1584 |
| Ile Val Asp Leu Trp Glu Gly Arg His Val Ala Arg Val Gly Pro Leu | |
| 490 495 500 505 | |
| ccg tgg ctc tgg gcg gcg cgg acg cgc gta gcg cgg gag cag ggc act | 1632 |
| Pro Trp Leu Trp Ala Ala Arg Thr Arg Val Ala Arg Glu Gln Gly Thr | |
| 510 515 520 | |
| gtg ctg ctg ctg tgg agc ggc gcc gac ctt cgc ccg gtc agc ggc ccc | 1680 |
| Val Leu Leu Leu Trp Ser Gly Ala Asp Leu Arg Pro Val Ser Gly Pro | |
| 525 530 535 | |
| gac ccc cgc gcc gcg ccc ctg ctc gcc ctg ctc cac gct gcc ccg cgc | 1728 |
| Asp Pro Arg Ala Ala Pro Leu Leu Ala Leu Leu His Ala Ala Pro Arg | |
| 540 545 550 | |
| ccg ctg ctg ctg ctc gct tac ttc agt cgc ctc tgc gcc aag ggc gac | 1776 |
| Pro Leu Leu Leu Leu Ala Tyr Phe Ser Arg Leu Cys Ala Lys Gly Asp | |
| 555 560 565 | |
| atc ccc ccg ccg ctg cgc gcc ctg ccg cgc tac cgc ctg ctg cgc gac | 1824 |
| Ile Pro Pro Pro Leu Arg Ala Leu Pro Arg Tyr Arg Leu Leu Arg Asp | |
| 570 575 580 585 | |
| ctg ccg cgt ctg ctg cgg gcg ctg gac gcg cgg cct ttc gca gag gcc | 1872 |
| Leu Pro Arg Leu Leu Arg Ala Leu Asp Ala Arg Pro Phe Ala Glu Ala | |
| 590 595 600 | |
| acc agc tgg ggc cgc ctt ggg gcg cgg cag cgc agg cag agc cgc cta | 1920 |
| Thr Ser Trp Gly Arg Leu Gly Ala Arg Gln Arg Arg Gln Ser Arg Leu | |
| 605 610 615 | |
| gag ctg tgc agc cgg ctc gaa cga gag gcc gcc cga ctt gca gac cta | 1968 |
| Glu Leu Cys Ser Arg Leu Glu Arg Glu Ala Ala Arg Leu Ala Asp Leu | |
| 620 625 630 | |
| ggt tgagcagagc tccaccgcag tcccgggtgt ctgcggccgc t | 2012 |
| Gly | |

MGSSRLAALLLPLLLIVIDLSDSAGIGFRHLPHWNTRCPLASHTEVLPISLAAPGGPSSPQSLGVCESGTVP
AVCASICCQVAQVFNGASSTSWCRNPKSLPHSSSIGDTRCQHLLRGSCCLVVTCLRRAITFPSPPQTSPTRD
FALKGPNLRIQRHGKVFPDWTHKGMEVGTGYNRRWVQLSGGPEFSFDLLPEARAIRVTISSGPEVSVRLCHQ
WALECEELSSPYDVQKIVSGGHTVELPYEFLLPCLCIEASYLQEDTVRRKKCPFQSWPEAYGSDFWKSVHFT
DYSQHTQMVMALTLRCPLKLEAALCQRHDWHTLCKDLPNATARESDGWYVLEKVDLHPQLCFKVQPWFSFGN
SSHVECPHQTGSLTSWNVSMDTQAQQLILHFSSRMHATFSAAWSLPGLGQDTLVPPVYTVSQVWRSDVQFAW
KHLLCPDVSYRHLGLLILALLALLTLLGVVLALTCRRPQSGPGPARPVLLLHAADSEAQRRLVGALAELLRA
ALGGGRDVIVDLWEGRHVARVGPLPWLWAARTRVAREQGTVLLLWSGADRPVSGPDPRAAPLLALLHAAPR
PLLLLAYFSRLCAKGDIPPPLRALPRYRLLRDLPRLLRALDARPFAEATSWGRLGARQRRQSRLELCSRLER
EAARLADLG.

!Reverse translation of primate, e.g., human, DCRS9 (SEQ ID NO:18):

| | |
|---|---|
| atgggnwsnw snmgnytngc ngcnytnytn ytnccnytny tnytnathgt nathgayytn | 60 |
| wsngaywsng cnggnathgg nttymgncay ytnccncayt ggaayacnmg ntgyccnytn | 120 |
| gcnwsncaya cngargtnyt nccnathwsn ytngcngcnc cnggnggncc nwsnwsnccn | 180 |
| carwsnytng gngtntgyga rwsnggnacn gtnccngdng tntgygcnws nathtgytgy | 240 |
| cargtngcnc argtnttyaa yggngcnwsn wsnacnwsnt ggtgymgnaa yccnaarwsn | 300 |
| ytnccncayw snwsnwsnat hggngayacn mgntgycarc ayytnytnmg nggnwsntgy | 360 |
| tgyytngtng tnacntgyyt nmgnmgngcn athacnttyc cnwsnccncc ncaracnwsn | 420 |
| ccnacnmgng ayttygcnyt naarggnccn aayytnmgna thcarmgnca yggnaargtn | 480 |
| ttyccngayt ggacncayaa rggnatggar gtnggnacng gntayaaymg nmgntgggtn | 540 |
| carytnwsng gnggnccnga rttywsntty gayytnytnc cngargcnmg ngcnathmgn | 600 |
| gtnacnathw snwsnggncc ngargtnwsn gtnmgnytnt gycaycartg ggcnytngar | 660 |
| tgygargary tnwsnwsncc ntaygaygtn caraarathg tnwsnggngg ncayacngtn | 720 |
| garytnccnt aygarttyyt nytnccntgy ytntgyathg argcnwsnta yytncargar | 780 |
| gayacngtnm gnmgnaaraa rtgyccntty carwsntggc cngargcnta yggnwsngay | 840 |
| ttytggaarw sngtncaytt yacngaytay wsncarcaya cncaratggt natggcnytn | 900 |
| acnytnmgnt gyccnytnaa rytngargcn gcnytntgyc armgncayga ytggcayacn | 960 |
| ytntgyaarg ayytnccriaa ygcnacngcn mgngarwsng ayggntggta ygtnytngar | 1020 |
| aargtngayy tncayccnca rytntgytty aargtncarc cntggttyws nttyggnaay | 1080 |
| wsnwsncayg tngartgycc ncaycaracn ggnwsnytna cnwsntggaa ygtnwsnatg | 1140 |
| gayacncarg cncarcaryt nathytncay ttywsnwsnm gnatgcaygc nadnttywsn | 1200 |
| gcngcntggw snytnccngg nytnggncar gayacnytng tnccnccngt ntayacngtn | 1260 |
| wsncargtnt ggmgnwsnga ygtncartty gcntggaarc ayytnytntg yccngaygtn | 1320 |
| wsntaymgnc ayytnggnyt nytnathytn gcnytnytng cnytnytnac nytnytnggn | 1380 |
| gtngtnytng cnytnacntg ymgnmgnccn carwsnggnc cnggnccngc nmgnccngtn | 1440 |
| ytnytnytnc aygcngcnga ywsngargcn carmgnmgny tngtnggngc nytngdngar | 1500 |
| ytnytnmgng dngcnytngg nggnggnmgn gaygtnathg tngayytntg ggarggnmgn | 1560 |
| caygtngcnm gngtnggncc nytncdntgg ytntgggcng cnmgnacnmg ngtngcnmgn | 1620 |
| garcarggna cngtnytnyt nytntggwsn ggngcngayy tnmgnccngt nwsnggnccn | 1680 |
| gayccnmgng cngcnccnyt nytngcnytn ytncaygcng cnccnmgncc nytnytnytn | 1740 |
| ytngcntayt tywsnmgnyt ntgygcnaar ggngayathc cnccnccnyt nmgngcnytn | 1800 |
| ccnmgntaym gnytnytnmg ngayytnccn mgnytnytnm gngcnytnga ygcnmgnccn | 1860 |
| ttygcngarg dnacnwsntg gggnmgnytn ggngcnmgnc armgnmgnca rwsnmgnytn | 1920 |
| garytntgyw snmgnytnga rmgngargcn gcnmgnytng cngayytngg n | 1971 |

!Rodent, e.g., mouse, embodiment (see SEQ ID NO: 19 and 20). Predicted? !signal sequence indicated, but may vary by a few positions and depending? !upon cell type.

| | |
|---|---|
| cagctccggg ccaggccctg ctgccctctt gcagacagga aagacatggt ctctgcgccc | 60 |
| tgatcctaca gaagctc atg ggg agc ccc aga ctg gca gcc ttg ctc ctg | 110 |
| Met Gly Ser Pro Arg Leu Ala Ala Leu Leu Leu | |
| −20 −15 | |
| tct ctc ccg cta ctg ctc atc ggc ctc gct gtg tct gct cgg gtt gcc | 158 |
| Ser Leu Pro Leu Leu Leu Ile Gly Leu Ala Val Ser Ala Arg Val Ala | |
| −10 −5 −1 1 | |
| tgc ccc tgc ctg cgg agt tgg acc agc cac tgt ctc ctg gcc tac cgt | 206 |
| Cys Pro Cys Leu Arg Ser Trp Thr Ser His Cys Leu Leu Ala Tyr Arg | |
| 5 10 15 20 | |
| gtg gat aaa cgt ttt gct ggc ctt cag tgg ggc tgg ttc cct ctc ttg | 254 |
| Val Asp Lys Arg Phe Ala Gly Leu Gln Trp Gly Trp Phe Pro Leu Leu | |
| 25 30 35 | |
| gtg agg aaa tct aaa agt cct cct aaa ttt gaa gac tat tgg agg cac | 302 |
| Val Arg Lys Ser Lys Ser Pro Pro Lys Phe Glu Asp Tyr Trp Arg His | |
| 40 45 50 | |
| agg aca cca gca tcc ttc cag agg aag ctg cta ggc agc cct tcc ctg | 350 |
| Arg Thr Pro Ala Ser Phe Gln Arg Lys Leu Leu Gly Ser Pro Ser Leu | |
| 55 60 65 | |
| tct gag gaa agc cat cga att tcc atc ccc tcc tca goc atc tcc cac | 398 |
| Ser Glu Glu Ser His Arg Ile Ser Ile Pro Ser Ser Ala Ile Ser His | |
| 70 75 80 | |
| aga ggc caa cgc acc aaa agg gcc cag cct tca got gca gaa gga aga | 446 |
| Arg Gly Gln Arg Thr Lys Arg Ala Gln Pro Ser Ala Ala Glu Gly Arg | |
| 85 90 95 100 | |
| gaa cat ctc cct gaa goa ggg tca caa aag tgt gga gga cct gaa ttc | 494 |
| Glu His Leu Pro Glu Ala Gly Ser Gln Lys Cys Gly Gly Pro Glu Phe | |
| 105 110 115 | |
| tcc ttt gat ttg ctg ccc gag gtg cag gct gtt cgg gtg act att cct | 542 |
| Ser Phe Asp Leu Leu Pro Glu Val Gln Ala Val Arg Val Thr Ile Pro | |
| 120 125 130 | |
| gca ggc ccc aag gca cgt gtg cgc ctt tgt tat cag tgg gca ctg gaa | 590 |
| Ala Gly Pro Lys Ala Arg Val Arg Leu Cys Tyr Gln Trp Ala Leu Glu | |
| 135 140 145 | |
| tgt gaa gac ttg agt agc cct ttt gat acc cag aaa att gtg tct gga | 638 |
| Cys Glu Asp Leu Ser Ser Pro Phe Asp Thr Gln Lys Ile Val Ser Gly | |
| 150 155 160 | |
| ggg cac act gta gac ctg cct tat gaa ttc ctt ctg ccc tgc atg tgc | 686 |
| Gly His Thr Val Asp Leu Pro Tyr Glu Phe Leu Leu Pro Cys Met Cys | |
| 165 170 175 180 | |
| ata gag gcc tcc tac ctg caa gag gac act gtg agg cgc aaa agt gtc | 734 |
| Ile Glu Ala Ser Tyr Leu Gln Glu Asp Thr Val Arg Arg Lys Ser Val | |
| 185 190 195 | |
| cct tcc aga gct ggc ctg aag ctt atg gct cag act tct ggc agt caa | 782 |
| Pro Ser Arg Ala Gly Leu Lys Leu Met Ala Gln Thr Ser Gly Ser Gln | |
| 200 205 210 | |
| tac gct tca ctg act aca gcc agc ac | 808 |

Tyr Ala Ser Leu Thr Thr Ala Ser
215 220
MGSPRLAALLLSLPLLLIGLAVSARVACPCLRSWTSHCLLAYRVDKRFAGLQWGWFPLLVRKSKSPPKFEDY
WRHRTPASFQRKLLGSPSLSEESHRISIPSSAISHRGQRTKRAQPSAAEGREHLPEAGSQKCGGPEFSFDLL
PEVQAVRVTIPAGPKAPVRLCYQWALECEDLSSPFDTQKIVSGGHTVDLPYEFLLPCMCIEASYLQEDTVRR
KSVPSRAGLKLMAQTSGSQYASLTTAS

!Reverse translation of rodent, e.g., mouse, DCRS9 (SEQ ID NO: 21):

| | |
|---|---|
| atgggnwsnc dnmgnytngc ngcnytnytn ytnwsnytnc cnytnytnyt nathggnytn | 60 |
| gcngtnwsng cnmgngtngc ntgyccntgy ytnmgnwsnt ggacnwsnca ytgyytnytn | 120 |
| gcntaymgng tngayaarmg nttygcnggn ytncartggg gntggttycc nytnytngtn | 180 |
| mgnaarwsna arwsnccncc naarttygar gaytaytggm gncaymgnac nccngcnwsn | 240 |
| ttycarmgna arytnytngg nwsnccnwsn ytnwsngarg arwsncaymg nathwsnath | 300 |
| ccnwsnwsng cnathwsnca ymgnggncar mgnacnaarm gngcncarcc nwsngcngcn | 360 |
| garggnmgng arcayytncc ngargcnggn wsncaraart gyggnggncc ngarttywsm | 420 |
| ttygayytny tnccngargt ncargcngtn mgngtnacna thccngcngg nccnaargcn | 480 |
| mgngtnmgny tntgytayca rtgggcnytn gartgygarg ayytnwsnws nccnttygay | 540 |
| acncaraara thgtnwsngg nggncayacn gtngayytnc dntaygartt yytnytnccn | 600 |
| tgyatgtgya thgargcnws ntayytncar gargayacng tnmgnmgnaa rwsngtnccn | 660 |
| wsnmgngcng gnytnaaryt natggdncar acnwsnggnw sncartaygc nwsnytnacn | 720 |
| acngcnwsn | 729 |

TABLE 5
Nucleotide and polypeptide sequences of DNAX Cytokine Receptor Subunit like
embodiments (DCRS10). Primate, e.g., human, embodiment
(see SEQ ID NO: 22 and 23).

| | |
|---|---|
| ttttgagcag aggcttccta ggctccgtag aaatttgcat acagcttcca cttcctgctt | 60 |
| cagagcctgt tcttctactt acctgggccc ggagaaggtg gagggagacg agaagccgcc | 120 |
| gagagccgac taccctccgg gcccagtctg tctgtccgtg gtggatctaa gaaactaga | 179 |
| atg aac cga agc att cct gtg gag gtt gat gaa tca gaa cca tac cca | 227 |
| Met Asn Arg Ser Ile Pro Val Glu Val Asp Glu Ser Glu Pro Tyr Pro | |
| 1 5 10 15 | |
| agt cag ttg ctg aaa cca atc cca gaa tat tcc ccg gaa gag gaa tca | 275 |
| Ser Gln Leu Leu Lys Pro Ile Pro Glu Tyr Ser Pro Glu Glu Glu Ser | |
| 20 25 30 | |
| gaa cca cct gct cca aat ata agg aac atg gca ccc aac agc ttg tct | 323 |
| Glu Pro Pro Ala Pro Asn Ile Arg Asn Met Ala Pro Asn Ser Leu Ser | |
| 35 40 45 | |
| gca ccc aca atg ctt cac aat tcc tcc gga gac ttt tct caa gct cac | 371 |
| Ala Pro Thr Met Leu His Asn Ser Ser Gly Asp Phe Ser Gln Ala His | |
| 50 55 60 | |
| tca acc ctg aaa ctt gca aat cac cag cgg cct gta tcc cgg cag gtc | 419 |
| Ser Thr Leu Lys Leu Ala Asn His Gln Arg Pro Val Ser Arg Gln Val | |
| 65 70 75 80 | |
| acc tgc ctg cgc act caa gtt ctg gag gac agt gaa gac agt ttc tgc | 467 |
| Thr Cys Leu Arg Thr Gln Val Leu Glu Asp Ser Glu Asp Ser Phe Cys | |
| 85 90 95 | |
| agg aga cac cca ggc ctg ggc aaa gct ttc cct tct ggg tgc tct gca | 515 |
| Arg Arg His Pro Gly Leu Gly Lys Ala Phe Pro Ser Gly Cys Ser Ala | |
| 100 105 110 | |
| gtc agc gag cct gcg tct gag tct gtg gtt gga gcc ctc cct gca gag | 563 |
| Val Ser Glu Pro Ala Ser Glu Ser Val Val Gly Ala Leu Pro Ala Glu | |
| 115 120 125 | |
| cat cag ttt tea ttt atg gaa aaa cgt aat caa tgg ctg gta tct cag | 611 |
| His Gln Phe Ser Phe Met Glu Lys Arg Asn Gln Trp Leu Val Ser Gln | |
| 130 135 140 | |
| ctt tca gcg gct tct cct gac act ggc cat gac tca gac aaa tca gac | 659 |
| Leu Ser Ala Ala Ser Pro Asp Thr Gly His Asp Ser Asp Lys Ser Asp | |
| 145 150 155 160 | |
| caa agt tta cct aat gcc tca gca gac tcc ttg ggc ggt agc cag gag | 707 |
| Gln Ser Leu Pro Asn Ala Ser Ala Asp Ser Leu Gly Gly Ser Gln Glu | |
| 165 170 175 | |
| atg gtg caa cgg ccc cag cct cac agg aac cga gca ggc ctg gat ctg | 755 |
| Met Val Gln Arg Pro Gln Pro His Arg Asn Arg Ala Gly Leu Asp Leu | |
| 180 185 190 | |
| cca acc ata gac acg gga tat gat tcc cag ccc cag gat gtc ctg ggc | 803 |
| Pro Thr Ile Asp Thr Gly Tyr Asp Ser Gln Pro Gln Asp Val Leu Gly | |
| 195 200 205 | |
| atc agg cag ctg gaa agg ccc ctg ccc ctc acc tcc gtg tgt tac ccc | 851 |
| Ile Arg Gln Leu Glu Arg Pro Leu Pro Leu Thr Ser Val Cys Tyr Pro | |
| 210 215 220 | |
| cag gac ctc ccc aga cct ctc agg tcc agg gag ttc cct cag ttt gaa | 899 |
| Gln Asp Leu Pro Arg Pro Leu Arg Ser Arg Glu Phe Pro Gln Phe Glu | |
| 225 230 235 240 | |
| cct cag agg tat cca gca tgt gca cag atg ctg cct ccc aat ctt tcc | 947 |
| Pro Gln Arg Tyr Pro Ala Cys Ala Gln Met Leu Pro Pro Asn Leu Ser | |
| 245 250 255 | |
| cca cat gct cca tgg aac tat cat tac cat tgt cct gga agt ccc gat | 995 |
| Pro His Ala Pro Trp Asn Tyr His Tyr His Cys Pro Gly Ser Pro Asp | |
| 260 265 270 | |
| cac cag gtg cca tat ggc cat gac tac cct cga gca gcc tac cag caa | 1043 |
| His Gln Val Pro Tyr Gly His Asp Tyr Pro Arg Ala Ala Tyr Gln Gln | |
| 275 280 285 | |
| gtg atc cag ccg gct ctg cct ggg cag ccc ctg cct gga gcc agt gtg | 1091 |
| Val Ile Gln Pro Ala Leu Pro Gly Gln Pro Leu Pro Gly Ala Ser Val | |
| 290 295 300 | |
| aga ggc ctg cac cct gtg cag aag gtt atc ctg aat tat ccc agc ccc | 1139 |
| Arg Gly Leu His Pro Val Gln Lys Val Ile Leu Asn Tyr Pro Ser Pro | |
| 305 310 315 320 | |
| tgg gac caa gaa gag agg ccc gca cag aga gac tgc tcc ttt ccg ggg | 1187 |
| Trp Asp Gln Glu Glu Arg Pro Ala Gln Arg Asp Cys Ser Phe Pro Gly | |
| 325 330 335 | |
| ctt cca agg cac cag gac cag cca cat cac cag cca cct aat aga gct | 1235 |
| Leu Pro Arg His Gln Asp Gln Pro His His Gln Pro Pro Asn Arg Ala | |
| 340 345 350 | |
| ggt gct cct ggg gag tcc ttg gag tgc cct gca gag ctg aga cca cag | 1283 |
| Gly Ala Pro Gly Glu Ser Leu Glu Cys Pro Ala Glu Leu Arg Pro Gln | |
| 355 360 365 | |
| gtt ccc cag cct ccg tcc cca gct gct gtg cct aga ccc cct agc aac | 1331 |
| Val Pro Gln Pro Pro Ser Pro Ala Ala Val Pro Arg Pro Pro Ser Asn | |
| 370 375 380 | |
| cct cca gcc aga gga act cta aaa aca agc aat ttg cca gaa gaa ttg | 1379 |
| Pro Pro Ala Arg Gly Thr Leu Lys Thr Ser Asn Leu Pro Glu Glu Leu | |
| 385 390 395 400 | |
| cgg aaa gtc ttt atc act tat tcg atg gac aca gct atg gag gtg gtg | 1427 |
| Arg Lys Val Phe Ile Thr Tyr Ser Met Asp Thr Ala Met Glu Val Val | |
| 405 410 415 | |
| aaa ttc gtg aac ttt ttg ttg gta aat ggc ttc caa act gca att gac | 1475 |
| Lys Phe Val Asn Phe Leu Leu Val Asn Gly Phe Gln Thr Ala Ile Asp | |
| 420 425 430 | |
| ata ttt gag gat aga atc cga ggc att gat atc att aaa tgg atg gag | 1523 |
| Ile Phe Glu Asp Arg Ile Arg Gly Ile Asp Ile Ile Lys Trp Met Glu | |
| 435 440 445 | |
| cgc tac ctt agg gat aag acc gtg atg ata atc gta gca atc agc ccc | 1571 |
| Arg Tyr Leu Arg Asp Lys Thr Val Met Ile Ile Val Ala Ile Ser Pro | |
| 450 455 460 | |
| aaa tac aaa cag gac gtg gaa ggc gct gag tcg cag ctg gac gag gat | 1619 |
| Lys Tyr Lys Gln Asp Val Glu Gly Ala Glu Ser Gln Leu Asp Glu Asp | |
| 465 470 475 480 | |
| gag cat ggc tta cat act aag tac att cat cga atg atg cag att gag | 1667 |
| Glu His Gly Leu His Thr Lys Tyr Ile His Arg Met Met Gln Ile Glu | |
| 485 490 495 | |
| ttc ata aaa caa gga agc atg aat ttc aga ttc atc cct gtg ctc ttc | 1715 |
| Phe Ile Lys Gln Gly Ser Met Asn Phe Arg Phe Ile Pro Val Leu Phe | |
| 500 505 510 | |
| cca aat gct aag aag gag cat gtg ccc acc tgg ctt cag aac act cat | 1763 |
| Pro Asn Ala Lys Lys Glu His Val Pro Thr Trp Leu Gln Asn Thr His | |
| 515 520 525 | |
| gtc tac agc tgg ccc aag aat aaa aaa aac atc ctg ctg cgg ctg ctg | 1811 |
| Val Tyr Ser Trp Pro Lys Asn Lys Lys Asn Ile Leu Leu Arg Leu Leu | |
| 530 535 540 | |
| aga gag gaa gag tat gtg gct cct cca cgg ggg cct ctg ccc acc ctt | 1859 |
| Arg Glu Glu Glu Tyr Val Ala Pro Pro Arg Gly Pro Leu Pro Thr Leu | |
| 545 550 555 560 | |
| cag gtg gtt ccc ttg tgacaccgtt catccccaga tcactgaggc caggccatgt | 1914 |
| Gln Val Val Pro Leu | |
| 565 | |
| ttggggcctt gttctgacag cattctggct gaggctggtc ggtagcactc ctggctggtt | 1974 |
| tttttctgtt cctccccgag aggccctctg gcccccagga aacctgttgt gcagagctct | 2034 |
| tccccggaga cctccacaca ccctggcttt gaagtggagt ctgtgactgc tctgcattct | 2094 |
| ctgcttttaa aaaaaccatt gcaggtgcca gtgtcccata tgttcctcct gacagtttga | 2154 |
| tgtgtccatt ctgggcctct cagtgcttag caagtagata atgtaaggga tgtggcagca | 2214 |
| aatggaaatg actacaaaca ctctcctatc aatcacttca ggctactttt atgagttagc | 2274 |
| cagatgcttg tgtatcctca gaccaaactg attcatgtac aaataataaa atgtttactc | 2334 |
| ttttgtaaaa aaaaaaaaaa aaaaaaaaag aaaaaaaaaa aaa | 2377 |
| MNRSIPVEVDESEPYPSQLLKPIPEYSPEEESEPPAPNIRNMAPNSLSAPTMLHNSSGDFSQAHSTLKLANH | |
| QRPVSRQVTCLRTQVLEDSEDSFCRRHPGLGKAFPSGCSAVSEPASESVVGALPAEHQFSFMEKRNQWLVSQ | |
| LSAASPDTGHDSDKSDQSLPNASADSLGGSQEMVQRPQPHRNRAGLDLPTIDTGYDSQPQDVLGIRQLERPL | |
| PLTSVCYPQDLPRPLRSREFPQFEPQRYPACAQMLPPNLSPHAPWNYHYHCPGSPDHQVPYGHDYPRAAYQQ | |
| VIQPALPGQPLPGASVRGLHPVQKVILNYPSPWDQEERPAQRDCSFPGLPRHQDQPHHQPPNRAGAPGESLE | |
| CPAELRPQVPQPPSPAAVPRPPSNPPARGTLKTSNLPEELRKVFITYSMDTAMEVVKFVNFLLVNGFQTAID | |
| IFEDRIRGTDTTKWMERYLRDKTVMTIVATSPKYKQDVEGAESQLDEDEHGLHTKYIHRMMQIEFTKQGSMN | |
| FRFIPVLFPNAKKEHVPTWLQNTHVYSWPKNKKNILLRLLREEEYVAPPRGPLPTLQVVPL | |

!Reverse translation of primate, e.g., human, DCRS10 (SEQ ID NO: 24):

| | |
|---|---|
| atgaaymgnw snathccngt ngargtngay garwsngarc cntayccnws ncarytnytn | 60 |
| aarccnathc cngartayws nccngargar garwsngarc cnccngcncc naayathmgn | 120 |
| aayatggcnc cnaaywsnyt nwsngcnccn acnatgytnc ayaaywsnws nggngaytty | 180 |
| wsncargcnc aywsnacnyt naarytngcn aaycaycarm gnccngtnws nmgncargtn | 240 |
| acntgyytnm gnacncargt nytngargay wsngargayw snttytgymg nmgncayccn | 300 |
| ggnytnggna argcnttycc nwsnggntgy wsngcngtnw sngarccngc nwsngarwsn | 360 |
| gtngtnggng cnytnccngc ngarcaycar ttywsnttya tggaraarmg naaycartgg | 420 |
| ytngtnwsnc arytnwsngc ngcnwsnccn gayacnggnc aygaywsnga yaarwsngay | 480 |
| carwsnytnc cnaaygcnws ngcngaywsn ytnggnggnw sncargarat ggtncarmgn | 540 |
| ccncarccnc aymgnaaymg ngcnggnytn gayytnccna cnathgayac nggntaygay | 600 |
| wsncarccnc argaygtnyt nggnathmgn carytngarm gnccnytncc nytnacnwsn | 660 |
| gtntgytayc cncargayyt nccnmgnccn ytnmgnwsnm gngarttycc ncarttygar | 720 |
| ccncarmgnt ayccngcntg ygcncaratg ytnccnccna ayytnwsncc ncaygcnccn | 780 |
| tggaaytayc aytaycaytg yccnggnwsn ccngaycayc argtnccnta yggncaygay | 840 |
| tayccnmgng cngcntayca rcargtnath carccngcny tnccnggnca rccnytnccn | 900 |
| ggngcnwsng tnmgnggnyt ncayccngtn caraargtna thytnaayta yccnwsnccn | 960 |
| tgggaycarg argarmgncc ngcncarmgn gaytgywsnt tyccnggnyt nccnmgncay | 1020 |
| cargaycarc cncaycayca rccnccnaay mgngcnggng cnccnggnga rwsnytngar | 1080 |
| tgyccngcng arytnmgncc ncargtnccn carccnccnw snccngcngc ngtnccnmgn | 1140 |
| ccnccnwsna ayccnccngc nmgnggnacn ytnaaracnw snaayytncc ngargarytn | 1200 |
| mgnaargtnt tyathacuta ywsnatggay acngcnatgg argtngtnaa rttygtnaay | 1260 |
| ttyytnytng tnaayggntt ycaradngcn athqayatht tygargaymg nathmgnggn | 1320 |
| athgayatha thaartggat ggarmgntay ytnmgngaya aracngtnat gathathgtn | 1380 |
| gcnathwsnc dnaartayaa rcargaygtn garggngcng arwsncaryt ngaygargay | 1440 |
| garcayggny tncayacnaa rtayathcay mgnatgatgc arathgartt yathaarcar | 1500 |
| ggnwsnatga ayttymgntt yathccngtn ytnttyccna aygcnaaraa rgarcaygtn | 1560 |
| ccnacntggy tncaraayac ncaygtntay wsntggccna araayaaraa raayathytn | 1620 |
| ytnmgnytny tnmgngarga rgartaygtn gcnccnccnm gnggnccnyt nccnacnytn | 1680 |
| cargtngtnc cnytn | 1695 |

Rodent, e.g., mouse, embodiment (see SEQ ID NO: 25 and 26).

| | |
|---|---|
| cag gac ctc cct ggg cct ctg agg tcc agg gaa ttg cca cct cag ttt | 48 |
| Gln Asp Leu Pro Gly Pro Leu Arg Ser Arg Glu Leu Pro Pro Gln Phe | |
| 1 5 10 15 | |
| gaa ctt gag agg tat cca atg aac gcc cag ctg ctg ccg ccc cat cct | 96 |
| Glu Leu Glu Arg Tyr Pro Met Asn Ala Gln Leu Leu Pro Pro His Pro | |
| 20 25 30 | |
| tcc cca cag gcc cca tgg aac tgt cag tac tac tgc ccc gga ggg ccc | 144 |
| Ser Pro Gln Ala Pro Trp Asn Cys Gln Tyr Tyr Cys Pro Gly Gly Pro | |
| 35 40 45 | |
| tac cac cac cag gtg cca cac ggc cat ggc tac cct cca gca gca gcc | 192 |
| Tyr His His Gln Val Pro His Gly His Gly Tyr Pro Pro Ala Ala Ala | |
| 50 55 60 | |
| tac cag caa gta ctc cag cct gct ctg cct ggg cag gtc ctt cct ggg | 240 |
| Tyr Gln Gln Val Leu Gln Pro Ala Leu Pro Gly Gln Val Leu Pro Gly | |
| 65 70 75 80 | |
| gca agg gca aga ggc cca cgc cct gtg cag aag gtc atc ctg aat gac | 288 |
| Ala Arg Ala Arg Gly Pro Arg Pro Val Gln Lys Val Ile Leu Asn Asp | |
| 85 90 95 | |
| tcc agc ccc caa gac caa gaa gag aga cct gca cag aga gac ttc tct | 336 |
| Ser Ser Pro Gln Asp Gln Glu Glu Arg Pro Ala Gln Arg Asp Phe Ser | |
| 100 105 110 | |
| ttc ccg agg ctc ccg agg gac cag ctc tac cgc cca cca tct aat gga | 384 |
| Phe Pro Arg Leu Pro Arg Asp Gln Leu Tyr Arg Pro Pro Ser Asn Gly | |
| 115 120 125 | |
| gtg gaa gcc cct gag gag tcc ttg gac ctt cct gca gag ctg aga cca | 432 |
| Val Glu Ala Pro Glu Glu Ser Leu Asp Leu Pro Ala Glu Leu Arg Pro | |
| 130 135 140 | |
| cat ggt ccc cag gct cca tcc cta gct gcc gtg cct aga ccc cct agc | 480 |
| His Gly Pro Gln Ala Pro Ser Leu Ala Ala Val Pro Arg Pro Pro Ser | |
| 145 150 155 160 | |
| aac ccc tta gcc cga gga act cta aga acc agc aat ttg cca gaa gaa | 528 |
| Asn Pro Leu Ala Arg Gly Thr Leu Arg Thr Ser Asn Leu Pro Glu Glu | |
| 165 170 175 | |
| tta cgg aaa gtc ttt atc act tat tct atg gac aca gcc atg gag gtg | 576 |
| Leu Arg Lys Val Phe Ile Thr Tyr Ser Met Asp Thr Ala Met Glu Val | |
| 180 185 190 | |
| gtg aaa ttt gtg aac ttt ctg ttg gtg aac ggc ttc caa act gcg att | 624 |
| Val Lys Phe Val Asn Phe Leu Leu Val Asn Gly Phe Gln Thr Ala Ile | |
| 195 200 205 | |
| gac ata ttt gag gat aga atc cgg ggt att gat atc att aaa tgg atg | 672 |
| Asp Ile Phe Glu Asp Arg Ile Arg Gly Ile Asp Ile Ile Lys Trp Met | |
| 210 215 220 | |
| gag cgc tat ctt cga gat aag aca gtg atg ata atc gta gca atc agc | 720 |
| Glu Arg Tyr Leu Arg Asp Lys Thr Val Met Ile Ile Val Ala Ile Ser | |
| 225 230 235 240 | |
| ccc aaa tac aaa cag gat gtg gaa ggc gct gag tcg cag ctg gac gag | 768 |
| Pro Lys Tyr Lys Gln Asp Val Glu Gly Ala Glu Ser Gln Leu Asp Glu | |
| 245 250 255 | |
| gac gag cat ggc tta cat act aag tac att cat cgg atg atg cag att | 816 |
| Asp Glu His Gly Leu His Thr Lys Tyr Ile His Arg Met Met Gln Ile | |
| 260 265 270 | |
| gag ttc ata agt cag gga agc atg aac ttc aga ttc atc cct gtg ctc | 864 |
| Glu Phe Ile Ser Gln Gly Ser Met Asn Phe Arg Phe Ile Pro Val Leu | |
| 275 280 285 | |
| ttc cca aat gcc aag aag gag cat gtg ccg acc tgg ctt cag aac act | 912 |
| Phe Pro Asn Ala Lys Lys Glu His Val Pro Thr Trp Leu Gln Asn Thr | |
| 290 295 300 | |
| cat gtt tac agc tgg ccc aag aat aag aaa aac atc ctg ctg cgg ctg | 960 |
| His Val Tyr Ser Trp Pro Lys Asn Lys Lys Asn Ile Leu Leu Arg Leu | |
| 305 310 315 320 | |
| ctc agg gag gaa gag tat gtg gct cct ccc cga ggc cct ctg ccc acc | 1008 |
| Leu Arg Glu Glu Glu Tyr Val Ala Pro Pro Arg Gly Pro Leu Pro Thr | |
| 325 330 335 | |
| ctt cag gtg gta ccc ttg tgacgatggc cactccagct cagtgccagc | 1056 |
| Leu Gln Val Val Pro Leu | |
| 340 | |
| ctgttctcac agcattcttc tagcggagct ggctggtggc acccaggccc tggaacacct | 1116 |
| cttctacaga gtcctctgtc tcctgagtct gagttgtcct cgctgggctt ccagagcttc | 1176 |
| agtgcctgga tgctgcaggt gacagaaaca aacatctatg accacaaaaa ctctcatcac | 1236 |
| ttcagctact tttatgagtc ggtcagatgc tctgtgtcct tagaccagtc taaatcatgc | 1296 |
| tcaaataata aaatgattat tctttgt | 1323 |
| QDLPGPLRSRELPPQFELERYPMNAQLLPPHPSPQAPWNCQYYCPGGPYHHQVPHGHGYPPAAAYQQVLQPA | |
| LPGQVLPGARARGPRPVQKVILNDSSPQDQEERPAQRDFSFPRLPRDQLYRPPSNGVEAPEESLDLPAELRP | |
| HGPQAPSLAAVPRPPSNPLARGTLRTSNLPEELRKVFITYSMDTAMEVVKFVNFLLVNGFQTAIDIFEDRIR | |
| GIDIIKWMERYLRDKTVMIIVAISPKYKQDVEGAESQLDEDEHGLHTKYIHRMMQIEFISQGSMNFRFIPVL | |
| FPNAKKEHVPTWLQNTHVYSWPKNKKNILLRLLREEEYVAPPRGPLPTLQVVPL. | |

Reverse translation of rodent, e.g., mouse, DCRS6 (SEQ ID NO: 27):

| | |
|---|---|
| cargayytnc cnggnccnyt nmgnwsnmgn garytnccnc cncarttyga rytngarmgn | 60 |
| tayccnatga aygcncaryt nytnccnccn cayccnwsnc cncargcncc ntggaaytgy | 120 |
| cartaytayt gyccnggngg nccntaycay caycargtnc cncayggnca yggntayccn | 180 |
| ccngcngcng cntaycarca rgtnytncar ccngcnytnc cnggncargt nytnccnggn | 240 |
| gcnmgngcnm gnggnccnmg nccngtncar aargtnathy tnaaygayws nwsnccncar | 300 |
| gaycargarg armgnccngc ncarmgngay ttywsnttyc cnmgnytncc nmgngaycar | 360 |
| ytntaymgnc cnccnwsnaa yggngtngar gcnccngarg arwsnytnga yytnccngcn | 420 |
| garytnmgnc cncayggncc ncargcnccn wsnytngcng cngtnccnmg nccnccnwsn | 480 |
| aayccnytng cnmgnggnac nytnmgnacn wsnaayytnc cngargaryt nmgnaargtn | 540 |
| ttyathacnt aywsnatgga yacngcnatg gargtngtna arttygtnaa yttyytnytn | 600 |
| gtnaayggnt tycaracngc nathgayath ttygargaym gnathmgngg nathgayath | 660 |
| athaartgga tggarmgnta yytnmgngay aaracngtna tgathathgt ngcnathwsn | 720 |
| ccnaartaya arcargaygt ngarggngcn garwsncary tngaygarga ygarcayggn | 780 |
| ytncayacna artayathca ymgnatgatg carathgart tyathwsnca rggnwsnatg | 840 |
| aayttymgnt tyathccngt nytnttyccn aaygcnaara argarcaygt nccnacntgg | 900 |
| ytncaraaya cncaygtnta ywsntggccn aaraayaara araayathyt nytnmgnytn | 960 |
| ytnmgngarg argartaygt ngcnccnccn mgnggnccny tnccnacnyt ncargtngtn | 1020 |
| ccnytn | 1026 |

TABLE 6

Alignment of the cytoplasmic portions of various cytokine receptor
subunits. The IL-17R_Hu (SEQ ID NO: 28) is GenBank AAB99730.1(U58917),
gi|7657230; the IL-17R_Mu (SEQ ID NO: 29) is GenBank
AAC52357.1(U31993), gi|6680411; the IL-17R_Ce (SEQ ID NO: 30) is
GenBankAAA811100.1(U39997), gi|1353171; and the DCRS6_Ce (SEQ ID NO:
31) is EMBCAA90543.1(Z50177), gi|7503597. Of particular interest are
motifs or features corresponding, in primate DCRS8 to: R/K at 339/340;
D/E at 348/349; alpha helical regions from H353-Q365, C370-S381,
E389-H396, K410-D414, and D485-H495; beta sheet regions correspond to
F400-V404 and F458-Y462; E at 431; E/D at 442/443; Y/F at 458; D/E at
468-470; Y/F at 481; and Q/R/F at 523.

| | |
|---|---|
| DCRS7_Mu | RTALLLHSADG-AGYERLVGALASALSQMP---LRVAVDLWSRRE-LSAHGALAWFHHQR |
| DCRS7_Hu | RAALLLYSADD-SGFERLVGALASALCQLP---LRVAVDLWSRRE-LSAQGPVAWFRAQR |
| IL-17R_Hu | RKVWIIYSADH-PLYVDVVLKFAQFLLTACG--TEVALDLLEEQA-ISEAGVMTWVGRQK |
| IL-17R_Mu | RKVWIVYSADH-PLYVEVVLKFAQFLITACG--TEVALDLLEEQV-ISEVGVMTWVSRQK |
| DCRS10 | RKVFTTYSMD----TAMEVVKFVNFLLVNG---FQTAIDIFEDR--IRGIDIIKWMERYL |
| DCRS10_Mu | RKVFITYSMD----TAMEVVKFVNFLLVNG---FQTAIDIFEDR--IRGIDIIKWMERYL |
| DCRS9_Hu | RPVLLLHAADS-EAQRRLVGALAELLRAALGGGRDVIVDLWEGRH-VARVGPLPWLWAAR |
| DCRS8_Hu | PKVFLCYSSKDGQNHMNVVQCFAYFLQDFCG--CEVALDLWEDFS-LCREGQREWVIQKI |
| IL-17R_Ce | VKVMIVYADDN-DLHTDCVKKLVENLRNCAS--CDPVFDLEKLI--TAEIVPSRWLVDQI |
| DCRS6_Hu | IKVLVVYPSEI--CFHHTICYFTEFLQNHCR--SEVILEKWQKKK-IAEMGPVQWLATQK |
| DCRS6_Ce | FKVMLVCPEVS-GRDEDFMMRIADALKKSN---NKVVCDRWFEDSKNAEENMLHWVYEQT |
| | . : . : :. * : *. |
| DCRS7_Mu | RRILQEGGVVILLFSPAAVAQCQ---QWLQLQTVEP---GP---HDALAAWLSCVLPDFL |
| DCRS7_Hu | RQTLQEGGVVVLLFSPGAVALCS---EWLQDGVSGPGARGP---HDAFPASLSCVLPDFL |
| IL-17R_Ru | QEMVESNSKIIVLCSRGTRAKWQALLGRGAP-VRLRCDHGKPV-GDLFTAAMNMILPDFK |
| IL-17R_Mu | QEMVESNSKIIILCSRGTQAKWKAILGWAEPAVQLRCDRWKPA-GDLFTAAMNMILPDFK |
| DCRS10 | R---DKTVMIIVAISPKYKQDVE----GAESQLDED-EHGL---HTKYIHRM-MQIEFIK |
| DCRS10_Mu | R---DKTVMIIVAISPKYKQDVE----GAESQLDED-EHGL---HTKYIHRM-MQIEFIS |
| DCRS9_Hu | TRVAREQGTVLLLWSGADLRPVS----GPDP-RAAP-----------LLA----LLHAAP |
| DCRS8_Hu | H----ESQFIIVVCSKGMKYFVD---KKNYKHKGGGRGSGK---GELFLVAVSAIAEKLR |
| IL-17R_Ce | S----SLKKFIIVVSDCAEKILD----TEASETHQLVQARP--FADLFGPANEMIIRDAT |
| DCRS6_Hu | K----AADKVVFLLSNDVNSVCD----GTCGKSEGSPSENS---QDLFPLAFNLFCSDLR |
| DCRS6_Ce | K----IAEKIIVFHSAYYHPRCG---IYDVINNFFPCTDPR-----LAHTALT---PEAQ |
| | .:. * |
| DCRS7_Mu | QGRATGR-----YVGVYFDGLLHPDSVPSPFRVAPLFSLP-SQLPAFLDALQ--GGCSTS |
| DCRS7_Hu | QGPAPGS-----YVGACFDRLLRPDAVPALFRTVPVFTLP-SQLPDFLGALQ--QPRAPR |
| IL-17R_Hu | RPACFGT-----YVVCYFSEVSCDGDVPDLFGAAPRYPLM-DRFEEVYFRIQ--DLEMFQ |
| IL-17R_Mu | RPACFGT-----YVVCYFSGICSERDVPDLFNITSRYPLM-DRFEEVYFRIQ--DLEMFE |
| DCRS10 | QGSMNFR-----FIPVLFPNAK-KEHVPTWLQNTHVYSWP-KNKKNILLRLL-REEEYVA |
| DCRS10_Mu | QGSMNFR-----FIPVLFPNAK-KEHVPTWLQNTHVYSWP-KNKKNILLRLL-REEEYVA |
| DCRS9_Hu | RPL---------LLLAYFSRLCAKGDIPPPLRALPRYRLL-RDLPRLLRALD--ARPFAE |
| DCRS8_Ru | QAKQSSSAALSKFIAVYFDYSC-EGDVPGTLDLSTKYRLM-DNLPQLCSRLRSRDRGLQE |
| IL-17R_Ce | RNFPEAR---KKYAVVRFNYSP---RVPPNLAILNLPTFIPEQFAQLTAFLRN-VERTER |
| DCRS6_Hu | SQIRLHK-----YVVVYFREID-TKDDYNALSVCPKYHLM-KDATAFCAELL---HVKQQ |
| DCRS6_Ce | RSVPKEV----EYVLPRDQKLL--EDAFDITIADPLVIDIPIEDVAIPENVP--IHHESC |
| | . : |
| DCRS7_Mu | AGRPADRVER-----VT----QALRSALDSCTS----------- |
| DCRS7_Hu | SGRLQERAEQ-----VS----RALQPALDSYFRPP--------- |
| IL-17R_Hu | PGRMRRVGELSGDNYLRS---PGGRQLRAALDRFRDWQVRCPDW |
| IL-17R_Mu | PGRMRRVRELTGDNYLQS---PSGRQLKEAVLRFQEWQTQCPDW |
| DCRS10 | P----PRGPL-----------PTLQVVPL--------------- |
| DCRS10_Mu | P----PRGPL-----------PTLQVVPL--------------- |
| DCRS9_Hu | ATSWGRLGAR-----------QRRQSRLELCSR----------- |
| DCRS8_Hu | PGQHTRQGSR-----RNYFRSKSGRSLYVAICNMHQFIDEEPDW |
| IL-17R_Ce | ANVTQNISEA------Q------IHEWNLCASRMMSFFVRNPNW |
| DCRS6_Hu | VS----AGKR-------------SQACRDGCCSL---------- |
| DCRS6_Ce | DSIDSRNNSK-------------THSTDSGVSSLSS----NS-- |
| | : |

Table 6 shows comparison of the available sequences of primate, rodent, and various other receptors. Various conserved residues are aligned and indicated. The structually homologous cytoplasmic domains most likely signal through pathways like IL-17, e.g., through NFKB. Similar to IL-1 signalling, it is likely that these receptors are invloved in innate immunity and/or development.

As used herein, the term DCRS shall be used to describe a protein comprising amino acid sequences shown in Tables 1-5, respectively. In many cases, a substantial fragment thereof will be functionally or structurally equivalent, including, e.g., an extracellular or intracellular domain. The invention also includes a protein variation of the respective DCRS allele whose sequence is provided, e.g., a mutein or soluble extracellular construct. Typically, such agonists or antagonists will exhibit less than about 10% sequence differences, and thus will often have between 1 and 11 substitutions, e.g., 2-, 3-, 5-, 7-fold, and others. It also encompasses allelic and other variants, e.g., natural polymorphic, of the protein described. Typically, it will bind to its corresponding biological ligand, perhaps in a dimerized state with an alpha receptor subunit, with high affinity, e.g., at least about 100 nM, usually better than about 30 nM, preferably better than about 10 nM, and more preferably at better than about 3 nM. The term shall also be used herein to refer to related naturally occurring forms, e.g., alleles, polymorphic variants, and metabolic variants of the mammalian protein. Preferred forms of the receptor complexes will bind the appropriate ligand with an affinity and selectivity appropriate for a ligand-receptor interaction.

This invention also encompasses combinations of proteins or peptides having substantial amino acid sequence identity with an amino acid sequence in Tables 1-5. It will include sequence variants with relatively few residue substitutions, e.g., preferably less than about 3-5.

A substantial polypeptide “fragment”, or “segment”, is a stretch of amino acid residues of at least about 8 amino acids, generally at least 10 amino acids, more generally at least 12 amino acids, often at least 14 amino acids, more often at least 16 amino acids, typically at least 18 amino acids, more typically at least 20 amino acids, usually at least 22 amino acids, more usually at least 24 amino acids, preferably at least 26 amino acids, more preferably at least 28 amino acids, and, in particularly preferred embodiments, at least about 30 or more amino acids. This includes, e.g., 40,50,60,70,85, 100, 115, 130, 150, and other lengths. Sequences of segments of different proteins can be compared to one another over appropriate length stretches, typically between conserved motifs. In many situations, fragments may exhibit functional properties of the intact subunits, e.g., the extracellular domain of the transmembrane receptor may retain the ligand binding features, and may be used to prepare a soluble receptor-like complex.

Amino acid sequence homology, or sequence identity, is determined by optimizing residue matches. In some comparisons, gaps may be introduces, as required. See, e.g., Needleham, et al., (1970) J. Mol. Biol. 48:443-453; Sankoff, et al., (1983) chapter one in Time Warps. String Edits. and Macromolecules: The Theory and Practice of Sequence Comparison. Addison-Wesley, Reading, MA; and software packages from IntelliGenetics, Mountain View, CA; and the University of Wisconsin Genetics Computer Group (GCG), Madison, WI; each of which is incorporated herein by reference. This changes when considering conservative substitutions as matches. Conservative substitutions typically include substitutions within the following groups: glycine, alanine; valine, isoleucine, leucine; aspartic acid, glutamic acid; asparagine, glutamine; serine, threonine; lysine, arginine; and phenylalanine, tyrosine. Homologous amino acid sequences are intended to include natural allelic and interspecies variations in the cytokine sequence. Typical homologous proteins or peptides will have from 50-100% homology (if gaps can be introduced), to 60-100% homology (if conservative substitutions are included) with an amino acid sequence segment of, e.g., Table 3 or 4. Homology measures will be at least about 70%, generally at least 76%, more generally at least 81%, often at least 85%, more often at least 88%, typically at least 90%, more typically at least 92%, usually at least 94%, more usually at least 95%, preferably at least 96%, and more preferably at least 97%, and in particularly preferred embodiments, at least 98% or more. The degree of homology will vary with the length of the compared segments. Homologous proteins or peptides, such as the allelic variants, will share most biological activities with the embodiments described in Tables 1-5.

As used herein, the term “biological activity” is used to describe, without limitation, effects on inflammatory responses, innate immunity, and/or morphogenic development by cytokine-like ligands. For example, these receptors should mediate phosphatase or phosphorylase activities, which activities are easily measured by standard procedures. See, e.g., Hardie, et al. (eds. 1995) The Protein Kinase FactBook vols. 1 and II, Academic Press, San Diego, Calif.; Hanks, et al. (1991) Meth. Enzymol. 200:38-62; Hunter, et al. (1992) Cell 70:375-388; Lewin (1990) Cell 61:743-752; Pines, et al. (1991) Cold Spring Harbor Symp. Quant. Biol. 56:449-463; and Parker, et al. (1993) Nature 363:736-738. The receptors, or portions thereof, may be useful as phosphate labeling enzymes to label general or specific substrates. The subunits may also be functional immunogens to elicit recognizing antibodies, or antigens capable of binding antibodies.

The terms ligand, agonist, antagonist, and analog of, e.g., a DCRS8 or DCRS9, include molecules that modulate the characteristic cellular responses to cytokine ligand proteins, as well as molecules possessing the more standard structural binding competition features of ligand-receptor interactions, e.g., where the receptor is a natural receptor or an antibody. The cellular responses likely are typically mediated through receptor tyrosine kinase pathways.

Also, a ligand is a molecule which serves either as a natural ligand to which said receptor, or an analog thereof, binds, or a molecule which is a functional analog of the natural ligand. The functional analog may be a ligand with structural modifications, or may be a wholly unrelated molecule which has a molecular shape which interacts with the appropriate ligand binding determinants. The ligands may serve as agonists or antagonists, see, e.g., Goodman, et al. (eds. 1990) Goodman & Gilnan's: The Pharmacological Bases of Therapeutics, Pergamon Press, N.Y.

Rational drug design may also be based upon structural studies of the molecular shapes of a receptor or antibody and other effectors or ligands. See, e.g., Herz, et al. (1997) J. Recept. Signal Transduct. Res. 17:671-776; and Chaiken, et al. (1996) Trends Biotechnol. 14:369-375. Effectors may be other proteins which mediate other functions in response to ligand binding, or other proteins which normally interact with the receptor. One means for determining which sites interact with specific other proteins is a physical structure determination, e.g., x-ray crystallography or 2 dimensional NMR techniques. These will provide guidance as to which amino acid residues form molecular contact regions. For a detailed description of protein structural determination, see, e.g., Blundell and Johnson (1976) Protein Crystallography, Academic Press, N.Y., which is hereby incorporated herein by reference.

### II. Activities

The cytokine receptor-like proteins will have a number of different biological activities, e.g., modulating cell proliferation, or in phosphate metabolism, being added to or removed from specific substrates, typically proteins. Such will generally result in modulation of an inflammatory function, other innate immunity response, or a morphological effect. The subunit will probably have a specific low affinity binding to the ligand.

The DCRS8 and DCRS9 have characteristic motifs of receptors signaling through the JAK pathway. See, e.g., Ihle, et al. (1997) Stem Cells 15(suppl. 1):105-111; Silvennoinen, et al. (1997) APMIS 105:497-509; Levy (1997) Cytokine Growth Factor Review 8:81-90; Winston and Hunter (1996) Current Biol. 6:668-671; Barrett (1996) Baillieres Clin. Gastroenterol. 10:1-15; and Briscoe, et al. (1996) Philos. Trans. R. Soc. Lond. B. Biol. Sci. 351:167-171.

The biological activities of the cytokine receptor subunits will be related to addition or removal of phosphate moieties to substrates, typically in a specific manner, but occasionally in a non specific manner. Substrates may be identified, or conditions for enzymatic activity may be assayed by standard methods, e.g., as described in Hardie, et al. (eds. 1995) The Protein Kinase FactBook vols. I and II, Academic Press, San Diego, CA; Hanks, et al. (1991) Meth. Enzymol. 200:38-62; Hunter, et al. (1992) Cell 70:375- 388; Lewin (1990) Cell 61:743-752; Pines, et al. (1991) Cold Spring Harbor Svmp. Quant. Biol. 56:449-463; and Parker, et al. (1993) Nature 363:736-738.

The receptor subunits may combine to form functional complexes, e.g., which may be useful for binding ligand or preparing antibodies. These will have substantial diagnostic uses, including detection or quantitation.

### III. Nucleic Acids

This invention contemplates use of isolated nucleic acid or fragments, e.g., which encode these or closely related proteins, or fragments thereof, e.g., to encode a corresponding polypeptide, preferably one which is biologically active. In addition, this invention covers isolated or recombinant DNAs which encode combinations of such proteins or polypeptides having characteristic sequences, e.g., of the DCRSs. Typically, the nucleic acid is capable of hybridizing, under appropriate conditions, with a nucleic acid sequence segment shown in Tables 1-5, but preferably not with a corresponding segment of other receptors described in Table 6. Said biologically active protein or polypeptide can be a full length protein, or fragment, and will typically have a segment of amino acid sequence highly homologous, e.g., exhibiting significant stretches of identity, to one shown in Tables 1-5. Further, this invention covers the use of isolated or recombinant nucleic acid, or fragments thereof, which encode proteins having fragments which are equivalent to the DCRS8 or DCRS9 proteins. The isolated nucleic acids can have the respective regulatory sequences in the 5′ and 3′ flanks, e.g., promoters, enhancers, poly-A addition signals, and others from the natural gene. Combinations, as described, are also provided.

An “isolated” nucleic acid is a nucleic acid, e.g., an RNA, DNA, or a mixed polymer, which is substantially pure, e.g., separated from other components which naturally accompany a native sequence, such as ribosomes, polymerases, and flanking genomic sequences from the originating species. The term embraces a nucleic acid sequence which has been removed from its naturally occurring environment, and includes recombinant or cloned DNA isolates, which are thereby distinguishable from naturally occurring compositions, and chemically synthesized analogs or analogs biologically synthesized by heterologous systems. A substantially pure molecule includes isolated forms of the molecule, either completely or substantially pure.

An isolated nucleic acid will generally be a homogeneous composition of molecules, but will, in some embodiments, contain heterogeneity, preferably minor. This heterogeneity is typically found at the polymer ends or portions not critical to a desired biological function or activity.

A “recombinant” nucleic acid is typically defined either by its method of production or its structure. In reference to its method of production, e.g., a product made by a process, the process is use of recombinant nucleic acid techniques, e.g., involving human intervention in the nucleotide sequence. Typically this intervention involves in vitro manipulation, although under certain circumstances it may involve more classical animal breeding techniques. Alternatively, it can be a nucleic acid made by generating a sequence comprising fusion of two fragments which are not naturally contiguous to each other, but is meant to exclude products of nature, e.g., naturally occurring mutants as found in their natural state. Thus, for example, products made by transforming cells with an unnaturally occurring vector is encompassed, as are nucleic acids comprising sequence derived using any synthetic oligonucleotide process. Such a process is often done to replace a codon with a redundant codon encoding the same or a conservative amino acid, while typically introducing or removing a restriction enzyme sequence recognition site. Alternatively, the process is performed to join together nucleic acid segments of desired fuinctions to generate a single genetic entity comprising a desired combination of functions not found in the commonly available natural forms, e.g., encoding a fusion protein. Restriction enzyme recognition sites are often the target of such artificial manipulations, but other site specific targets, e.g., promoters, DNA replication sites, regulation sequences, control sequences, or other useful features may be incorporated by design. A similar concept is intended for a recombinant, e.g., fusion, polypeptide. This will include a dimeric repeat. Specifically included are synthetic nucleic acids which, by genetic code redundancy, encode equivalent polypeptides to fragments of DCRSs and fusions of sequences from various different related molecules, e.g., other cytokine receptor family members.

A “fragment” in a nucleic acid context is a contiguous segment of at least about 17 nucleotides, generally at least 21 nucleotides, more generally at least 25 nucleotides, ordinarily at least 30 nucleotides, more ordinarily at least 35 nucleotides, often at least 39 nucleotides, more often at least 45 nucleotides, typically at least 50 nucleotides, more typically at least 55 nucleotides, usually at least 60 nucleotides, more usually at least 66 nucleotides, preferably at least 72 nucleotides, more preferably at least 79 nucleotides, and in particularly preferred embodiments will be at least 85 or more nucleotides. Typically, fragments of different genetic sequences can be compared to one another over appropriate length stretches, particularly defined segments such as the domains described below.

A nucleic acid which codes for the DCRS8 or DCRS9 will be particularly useful to identify genes, mRNA, and CDNA species which code for itself or closely related proteins, as well as DNAs which code for polymorphic, allelic, or other genetic variants, e.g., from different individuals or related species. Preferred probes for such screens are those regions of the interleukin which are conserved between different polymorphic variants or which contain nucleotides which lack specificity, and will preferably be fall length or nearly so. In other situations, polymorphic variant specific sequences will be more useful.

This invention further covers recombinant nucleic acid molecules and fragments having a nucleic acid sequence identical to or highly homologous to the isolated DNA set forth herein. In particular, the sequences will often be operably linked to DNA segments which control transcription, translation, and DNA replication. These additional segments typically assist in expression of the desired nucleic acid segment.

Homologous, or highly identical, nucleic acid sequences, when compared to one another, e.g., DCRS8 sequences, exhibit significant similarity. The standards for homology in nucleic acids are either measures for homology generally used in the art by sequence comparison or based upon hybridization conditions. Comparative hybridization conditions are described in greater detail below.

Substantial identity in the nucleic acid sequence comparison context means either that the segments, or their complementary strands, when compared, are identical when optimally aligned, with appropriate nucleotide insertions or deletions, in at least about 60% of the nucleotides, generally at least 66%, ordinarily at least 71%, often at least 76%, more often at least 80%, usually at least 84%, more usually at least 88%, typically at least 91%, more typically at least about 93%, preferably at least about 95%, more preferably at least about 96 to 98% or more, and in particular embodiments, as high at about 99% or more of the nucleotides, including, e.g., segments encoding structural domains such as the segments described below. Alternatively, substantial identity will exist when the segments will hybridize under selective hybridization conditions, to a strand or its complement, typically using a sequence derived from Tables 1-5. Typically, selective hybridization will occur when there is at least about 55% homology over a stretch of at least about 14 nucleotides, more typically at least about 65%, preferably at least about 75%, and more preferably at least about 90%. See, Kanehisa (1984) Nucl. Acids Res. 12:203-213, which is incorporated herein by reference. The length of homology comparison, as described, may be over longer stretches, and in certain embodiments will be over a stretch of at least about 17 nucleotides, generally at least about 20 nucleotides, ordinarily at least about 24 nucleotides, usually at least about 28 nucleotides, typically at least about 32 nucleotides, more typically at least about 40 nucleotides, preferably at least about 50 nucleotides, and more preferably at least about 75 to 100 or more nucleotides. This includes, e.g., 125, 150, 175, 200, 225, 246, 273, and other lengths.

Stringent conditions, in referring to homology in the hybridization context, will be stringent combined conditions of salt, temperature, organic solvents, and other parameters typically controlled in hybridization reactions. Stringent temperature conditions will usually include temperatures in excess of about 30 C, more usually in excess of about 37 C, typically in excess of about 45 C, more typically in excess of about 55 C, preferably in excess of about 65 C, and more preferably in excess of about 70 C. Stringent salt conditions will ordinarily be less than about 500 mM, usually less than about 400 mM, more usually less than about 300 mM, typically less than about 200 MM, preferably less than about 100 mM, and more preferably less than about 80 mlM, even down to less than about 20 mM. However, the combination of parameters is much more important than the measure of any single parameter. See, e.g., Wetmur and Davidson (1968) J. Mol. Biol. 31:349-370, which is hereby incorporated herein by reference.

The isolated DNA can be readily modified by nucleotide substitutions, nucleotide deletions, nucleotide insertions, and inversions of nucleotide stretches. These modifications result in novel DNA sequences which encode this protein or its derivatives. These modified sequences can be used to produce mutant proteins (muteins) or to enhance the expression of variant species. Enhanced expression may involve gene amplification, increased transcription, increased translation, and other mechanisms. Such mutant DCRS8-like derivatives include predetermined or site-specific mutations of the protein or its fragments, including silent mutations using genetic code degeneracy. “Mutant DCRS8” as used herein encompasses a polypeptide otherwise falling within the homology definition of the DCRS8 as set forth above, but having an amino acid sequence which differs from that of other cytokine receptor-like proteins as found in nature, whether by way of deletion, substitution, or insertion. In particular, “site specific mutant DCRS8” encompasses a protein having substantial sequence identity with a protein of Table 3, and typically shares most of the biological activities or effects of the forms disclosed herein.

Although site specific mutation sites are predetermined, mutants need not be site specific. Mammalian DCRS8 mutagenesis can be achieved by making amino acid insertions or deletions in the gene, coupled with expression. Substitutions, deletions, insertions, or many combinations may be generated to arrive at a final construct. Insertions include amino- or carboxy- terminal fusions. Random mutagenesis can be conducted at a target codon and the expressed mammalian DCRS mutants can then be screened for the desired activity, providing some aspect of a structure-activity relationship. Methods for making substitution mutations at predetermined sites in DNA having a known sequence are well known in the art, e.g., by M13 primer mutagenesis.

See also Sambrook, et al. (1989) and Ausubel, et al. (1987 and periodic Supplements).

The mutations in the DNA normally should not place coding sequences out of reading frames and preferably will not create complementary regions that could hybridize to produce secondary MRNA structure such as loops or hairpins.

The phosphoramidite method described by Beaucage and Carruthers (1981) Tetra.

Letts. 22:1859-1862, will produce suitable synthetic DNA fragments. A double stranded fragment will often be obtained either by synthesizing the complementary strand and annealing the strand together under appropriate conditions or by adding the complementary strand using DNA polymerase with an appropriate primer sequence.

Polymerase chain reaction (PCR) techniques can often be applied in mutagenesis.

Alternatively, mutagenesis primers are commonly used methods for generating defined mutations at predetermined sites. See, e.g., Innis, et al. (eds. 1990) PCR Protocols: A Guide to Methods and Applications Academic Press, San Diego, CA; and Dieffenbach and Dveksler (1995; eds.) PCR Primer: A Laboratorv Manual Cold Spring Harbor Press, CSH, N.Y.

Certain embodiments of the invention are directed to combination compositions comprising the receptor or ligand sequences described. In other embodiments, finctional portions of the sequences may be joined to encode fusion proteins. In other forms, variants of the described sequences may be substituted.

### IV. Proteins, Peptides

As described above, the present invention encompasses primate DCRS6-10, e.g., whose sequences are disclosed in Tables 1-5, and described above. Allelic and other variants are also contemplated, including, e.g., fusion proteins combining portions of such sequences with others, including, e.g., epitope tags and functional domains.

The present invention also provides recombinant proteins, e.g., heterologous fusion proteins using segments from these primate or rodent proteins. A heterologous fusion protein is a fusion of proteins or segments which are naturally not normally fused in the same manner. Thus, the fusion product of, e.g., a DCRS8 with another cytokine receptor is a continuous protein molecule having sequences fused in a typical peptide linkage, typically made as a single translation product and exhibiting properties, e.g., sequence or antigenicity, derived from each source peptide. A similar concept applies to beterologous nucleic acid sequences. Combinations of various designated proteins into complexes are also provided.

In addition, new constructs may be made from combining similar functional or structural domains from other related proteins, e.g., cytokine receptors or Toll-like receptors, including species variants. For example, ligand-binding or other segments may be “swapped” between different new fusion polypeptides or fragments. See, e.g., Cunningham, et al. (1989) Science 243:1330-1336; and O'Dowd, et al. (1988) J. Biol. Chem. 263:15985-15992, each of which is incorporated herein by reference. Thus, new chimeric polypeptides exhibiting new combinations of specificities will result from the finctional linkage of receptor-binding specificities. For example, the ligand binding domains from other related receptor molecules may be added or substituted for other domains of this or related proteins. The resulting protein will often have hybrid function and properties. For example, a fusion protein may include a targeting domain which may serve to provide sequestering of the fusion protein to a particular subcellular organelle.

Candidate fusion partners and sequences can be selected from various sequence data bases, e.g., GenBank, c/o IntelliGenetics, Mountain View, CA; and BCG, University of Wisconsin Biotechnology Computing Group, Madison, Wis., which are each incorporated herein by reference. In particular, combinations of polypeptide sequences provided in Tables 1-5 are particularly preferred. Variant forms of the proteins may be substituted in the described combinations.

The present invention particularly provides muteins which bind cytokine-like ligands, and/or which are affected in signal transduction. Structural alignment of human DCRSs with other members of the cytokine receptor family show conserved features/residues. See Table 6. Alignment of the human DCRS8 sequence with other members of the cytokine receptor family indicates various structural and functionally shared features. See also, Bazan, et al. (1996) Nature 379:591; Lodi, et al. (1994) Science 263:1762-1766; Sayle and Milner-White (1995) TIBS 20:374-376; and Gronenberg, et al. (1991) Protein Engineering 4:263-269.

Substitutions with either mouse sequences or human sequences are particularly preferred. Conversely, conservative substitutions away from the ligand binding interaction regions will probably preserve most signaling activities; and conservative substitutions away from the intracellular domains will probably preserve most ligand binding properties.

“Derivatives” of the primate DCRS8 include amino acid sequence mutants, glycosylation variants, metabolic derivatives and covalent or aggregative conjugates with other chemical moieties. Covalent derivatives can be prepared by linkage of functionalities to groups which are found in the DCRS8 amino acid side chains or at the N- or C- termin, e.g., by means which are well known in the art. These derivatives can include, without limitation, aliphatic esters or amides of the carboxyl terminus, or of residues containing carboxyl side chains, 0-acyl derivatives of hydroxyl group-containing residues, and N-acyl derivatives of the amino terminal amino acid or amino-group containing residues, e.g., lysine or arginine. Acyl groups are selected from the group of alkyl-moieties, including C3 to C18 normal alkyl, thereby forming alkanoyl aroyl species.

In particular, glycosylation alterations are included, e.g., made by modifying the glycosylation patterns of a polypeptide during its synthesis and processing, or in further processing steps. Particularly preferred means for accomplishing this are by exposing the polypeptide to glycosylating enzymes derived from cells which normally provide such processing, e.g., mammalian glycosylation enzymes. Deglycosylation enzymes are also contemplated. Also embraced are versions of the same primary amino acid sequence which have other minor modifications, including phosphorylated amino acid residues, e.g., phosphotyrosine, phosphoserine, or phosphothreonine.

A major group of derivatives are covalent conjugates of the receptors or fragments thereof with other proteins of polypeptides. These derivatives can be synthesized in recombinant culture such as N- or C-terminal fusions or by the use of agents known in the art for their usefulness in cross-linking proteins through reactive side groups. Preferred derivatization sites with cross-linking agents are at free amino groups, carbohydrate moieties, and cysteine residues.

Fusion polypeptides between the receptors and other homologous or heterologous proteins are also provided. Homologous polypeptides may be fusions between different receptors, resulting in, for instance, a hybrid protein exhibiting binding specificity for multiple different cytokine ligands, or a receptor which may have broadened or weakened specificity of substrate effect. Likewise, heterologous fusions may be constructed which would exhibit a combination of properties or activities of the derivative proteins. Typical examples are fusions of a reporter polypeptide, e.g., luciferase, with a segment or domain of a receptor, e.g., a ligand-binding segment, so that the presence or location of a desired ligand may be easily determined. See, e.g., Dull, et al., U.S. Patent No. 4,859,609, which is hereby incorporated herein by reference. Other gene fusion partners include glutathione-S-transferase (GST), bacterial B-galactosidase, trpE, Protein A, B-lactamase, alpha amylase, alcohol dehydrogenase, and yeast alpha mating factor. See, e.g., Godowski, et al. (1988) Science 241:812-816. Labeled proteins will often be substituted in the described combinations of proteins.

The phosphoramidite method described by Beaucage and Carruthers (1981) Tetra. Letts. 22:1859-1862, will produce suitable synthetic DNA fragments. A double stranded fragment will often be obtained either by synthesizing the complementary strand and annealing the strand together under appropriate conditions or by adding the complementary strand using DNA polymerase with an appropriate primer sequence.

Such polypeptides may also have amino acid residues which have been chemically modified by phosphorylation, sulfonation, biotinylation, or the addition or removal of other moieties, particularly those which have molecular shapes similar to phosphate groups. In some embodiments, the modifications will be useful labeling reagents, or serve as purification targets, e.g., affinity ligands.

Fusion proteins will typically be made by either recombinant nucleic acid methods or by synthetic polypeptide methods. Techniques for nucleic acid manipulation and expression are described generally, for example, in Sambrook, et al. (1989) Molecular Cloning: A Laboratory Manual (2d ed.), Vols. 1-3, Cold Spring Harbor Laboratory, and Ausubel, et al. (eds. 1987 and periodic supplements) Current Protocols in Molecular Biology, Greene/Wiley, N.Y., which are each incorporated herein by reference. Techniques for synthesis of polypeptides are described, for example, in Merrifield (1963) J. Amer. Chem. Soc. 85:2149-2156; Merrifield (1986) Science 232: 341-347; and Atherton, et al. (1989) Solid Phase Peptide Synthesis: A Practical Approach, IRL Press, Oxford; each of which is incorporated herein by reference. See also Dawson, et al. (1994) Science 266:776-779 for methods to make larger polypeptides.

This invention also contemplates the use of derivatives of a DCRS8 other than variations in amino acid sequence or glycosylation. Such derivatives may involve covalent or aggregative association with chemical moieties. These derivatives generally fall into three classes: (1) salts, (2) side chain and terminal residue covalent modifications, and (3) adsorption complexes, for example with cell membranes. Such covalent or aggregative derivatives are useful as immunogens, as reagents in immunoassays, or in purification methods such as for affinity purification of a receptor or other binding molecule, e.g., an antibody. For example, a cytokine ligand can be immobilized by covalent bonding to a solid support such as cyanogen bromide-activated Sepharose, by methods which are well known in the art, or adsorbed onto polyolefin surfaces, with or without glutaraldehyde cross-linking, for use in the assay or purification of a cytokine receptor, antibodies, or other similar molecules. The ligand can also be labeled with a detectable group, for example radioiodinated by the chloramine T procedure, covalently bound to rare earth chelates, or conjugated to another fluorescent moiety for use in diagnostic assays.

A combination, e.g., including a DCRS8, of this invention can be used as an immunogen for the production of antisera or antibodies specific, e.g., capable of distinguishing between other cytokine receptor family members, for the combinations described. The complexes can be used to screen monoclonal antibodies or antigen- binding fragments prepared by immunization with various forms of impure preparations containing the protein. In particular, the term “antibodies” also encompasses antigen binding fragments of natural antibodies, e.g., Fab, Fab2, Fv, etc. The purified DCRS8 can also be used as a reagent to detect antibodies generated in response to the presence of elevated levels of expression, or immunological disorders which lead to antibody production to the endogenous receptor. Additionally, DCRS8 fragments may also serve as immunogens to produce the antibodies of the present invention, as described immediately below. For example, this invention contemplates antibodies having binding affinity to or being raised against the amino acid sequences shown in Tables 1-5, fragments thereof, or various homologous peptides. In particular, this invention contemplates antibodies having binding affinity to, or having been raised against, specific fragments which are predicted to be, or actually are, exposed at the exterior protein surface of the native DCRS8 or DCRS9. Complexes of combinations of proteins will also be useful, and antibody preparations thereto can be made.

The blocking of physiological response to the receptor ligands may result from the inhibition of binding of the ligand to the receptor, likely through competitive inhibition. Thus, in vitro assays of the present invention will often use antibodies or antigen binding segments of these antibodies, or fragments attached to solid phase substrates. These assays will also allow for the diagnostic determination of the effects of either ligand binding region mutations and modifications, or other mutations and modifications, e.g., which affect signaling or enzymatic function.

This invention also contemplates the use of competitive drug screening assays, e.g., where neutralizing antibodies to the receptor complexes or fragments compete with a test compound for binding to a ligand or other antibody. In this manner, the neutralizing antibodies or fragments can be used to detect the presence of a polypeptide which shares one or more binding sites to a receptor and can also be used to occupy binding sites on a receptor that might otherwise bind a ligand. V. Making Nucleic Acids and Protein DNA which encodes the protein or fragments thereof can be obtained by chemical synthesis, screening cDNA libraries, or by screening genomic libraries prepared from a wide variety of cell lines or tissue samples. Natural sequences can be isolated using standard methods and the sequences provided herein, e.g., in Tables 1-5. Other species counterparts can be identified by hybridization techniques, or by various PCR techniques, combined with or by searching in sequence databases, e.g., GenBank.

This DNA can be expressed in a wide variety of host cells for the synthesis of a full-length receptor or fragments which can in turn, for example, be used to generate polyclonal or monoclonal antibodies; for binding studies; for construction and expression of modified ligand binding or kinase/phosphatase domains; and for structure/function studies. Variants or fragments can be expressed in host cells that are transformed or transfected with appropriate expression vectors. These molecules can be substantially free of protein or cellular contaminants, other than those derived from the recombinant host, and therefore are particularly useful in pharmaceutical compositions when combined with a pharmaceutically acceptable carrier and/or diluent. The protein, or portions thereof, may be expressed as fusions with other proteins. Combinations of the described proteins, or nucleic acids encoding them, are particularly interesting.

Expression vectors are typically self-replicating DNA or RNA constructs containing the desired receptor gene or its fragments, usually operably linked to suitable genetic control elements that are recognized in a suitable host cell. These control elements are capable of effecting expression within a suitable host. The multiple genes may be coordinately expressed, and may be on a polycistronic message. The specific type of control elements necessary to effect expression will depend upon the eventual host cell used. Generally, the genetic control elements can include a prokaryotic promoter system or a eukaryotic promoter expression control system, and typically include a transcriptional promoter, an optional operator to control the onset of transcription, transcription enhancers to elevate the level of mRNA expression, a sequence that encodes a suitable ribosome binding site, and sequences that terminate transcription and translation. Expression vectors also usually contain an origin of replication that allows the vector to replicate independently of the host cell.

The vectors of this invention include those which contain DNA which encodes a combination of proteins, as described, or a biologically active equivalent polypeptide. The DNA can be under the control of a viral promoter and can encode a selection marker. This invention further contemplates use of such expression vectors which are capable of expressing eukaryotic cDNAs coding for such proteins in a prokaryotic or eukaryotic host, where the vector is compatible with the host and where the eukaryotic cDNAs are inserted into the vector such that growth of the host containing the vector expresses the cDNAs in question. Usually, expression vectors are designed for stable replication in their host cells or for amplification to greatly increase the total number of copies of the desirable gene per cell. It is not always necessary to require that an expression vector replicate in a host cell, e.g., it is possible to effect transient expression of the protein or its fragments in various hosts using vectors that do not contain a replication origin that is recognized by the host cell. It is also possible to use vectors that cause integration of the protein encoding portions into the host DNA by recombination.

Vectors, as used herein, comprise plasmids, viruses, bacteriophage, integratable DNA fragments, and other vehicles which enable the integration of DNA fragments into the genome of the host. Expression vectors are specialized vectors which contain genetic control elements that effect expression of operably linked genes. Plasmids are the most commonly used form of vector but all other forms of vectors which serve an equivalent function and which are, or become, known in the art are suitable for use herein. See, e.g., Pouwels, et al. (1985 and Supplements) Cloning Vectors: A Laboratorv Manual, Elsevier, N.Y., and Rodriguez, et al. (eds. 1988) Vectors: A Survey of Molecular Cloning Vectors and Their Uses, Buttersworth, Boston, which are incorporated herein by reference.

Transformed cells are cells, preferably mammalian, that have been transformed or transfected with vectors constructed using recombinant DNA techniques. Transformed host cells usually express the desired proteins, but for purposes of cloning, amplifying, and manipulating its DNA, do not need to express the subject proteins. This invention further contemplates culturing transformed cells in a nutrient medium, thus permitting the proteins to accumulate. The proteins can be recovered, either from the culture or, in certain instances, from the culture medium.

For purposes of this invention, nucleic sequences are operably linked when they are functionally related to each other. For example, DNA for a presequence or secretory leader is operably linked to a polypeptide if it is expressed as a preprotein or participates in directing the polypeptide to the cell membrane or in secretion of the polypeptide. A promoter is operably linked to a coding sequence if it controls the transcription of the polypeptide; a ribosome binding site is operably linked to a coding sequence if it is positioned to permit translation. Usually, operably linked means contiguous and in reading frame, however, certain genetic elements such as repressor genes are not contiguously linked but still bind to operator sequences that in turn control expression.

Suitable host cells include prokaryotes, lower eukaryotes, and higher eukaryotes. Prokaryotes include both gram negative and gram positive organisms, e.g., E. coli and B. subtilis. Lower eukaryotes include yeasts, e.g., S. cerevisiae and Pichia, and species of the genus Dictyostelium. Higher eukaryotes include established tissue culture cell lines from animal cells, both of non-mammalian origin, e.g., insect cells, and birds, and of mammalian origin, e.g., human, primates, and rodents.

Prokaryotic host-vector systems include a wide variety of vectors for many different species. As used herein, E. coli and its vectors will be used generically to include equivalent vectors used in other prokaryotes. A representative vector for amplifying DNA is pBR322 or many of its derivatives. Vectors that can be used to express the receptor or its fragments include, but are not limited to, such vectors as those containing the lac promoter (pUC-series); trp promoter (pBR322-trp); Ipp promoter (the pIN-series); lambda-pP or pR promoters (pOTS); or hybrid promoters such as ptac (pDR540). See Brosius, et al. (1988) “Expression Vectors Employing Lambda-, trp-, lac-, and Ipp-derived Promoters”, in Vectors: A Survey of Molecular Cloning Vectors and Their Uses, (eds. Rodriguez and Denhardt), Buttersworth, Boston, Chapter 10, pp. 205-236, which is incorporated herein by reference.

Lower eukaryotes, e.g., yeasts and Dictyostelium, may be transformed with DCRS8 sequence containing vectors. For purposes of this invention, the most common lower eukaryotic host is the baker's yeast, Saccharomyces cerevisiae. It will be used to generically represent lower eukaryotes although a number of other strains and species are also available. Yeast vectors typically consist of a replication origin (unless of the integrating type), a selection gene, a promoter, DNA encoding the receptor or its fragments, and sequences for translation termination, polyadenylation, and transcription termination. Suitable expression vectors for yeast include such constitutive promoters as 3-phosphoglycerate kinase and various other glycolytic enzyme gene promoters or such inducible promoters as the alcohol dehydrogenase 2 promoter or metallothionine promoter. Suitable vectors include derivatives of the following types: self-replicating low copy number (such as the YRp-series), self-replicating high copy number (such as the YEp-series); integrating types (such as the Yip-series), or mini-chromosomes (such as the YCp-series).

Higher eukaryotic tissue culture cells are normally the preferred host cells for expression of the functionally active interleukin or receptor proteins. In principle, many higher eukaryotic tissue culture cell lines are workable, e.g., insect baculovirus expression systems, whether from an invertebrate or vertebrate source. However, mammalian cells are preferred. Transformation or transfection and propagation of such cells has become a routine procedure. Examples of useful cell lines include HeLa cells, Chinese hamster ovary (CHO) cell lines, baby rat kidney (BRK) cell lines, insect cell lines, bird cell lines, and monkey (COS) cell lines. Expression vectors for such cell lines usually include an origin of replication, a promoter, a translation initiation site, RNA splice sites (if genomic DNA is used), a polyadenylation site, and a transcription termination site. These vectors also usually contain a selection gene or amplification gene. Suitable expression vectors may be plasmids, viruses, or retroviruses carrying promoters derived, e.g., from such sources as from adenovirus, SV40, parvoviruses, vaccinia virus, or cytomegalovirus. Representative examples of suitable expression vectors include pCDNAI; pCD, see Okayama, et al. (1985) Mol. Cell Biol. 5:1136-1142; pMClneo PolyA, see Thomas, et al. (1987) Cell 51:503-512; and a baculovirus vector such as pAC 373 or pAC 610.

For secreted proteins and some membrane proteins, an open reading frame usually encodes a polypeptide that consists of a mature or secreted product covalently linked at its N-terminus to a signal peptide. The signal peptide is cleaved prior to secretion of the mature, or active, polypeptide. The cleavage site can be predicted with a high degree of accuracy from empirical rules, e.g., von-Heijne (1986) Nucleic Acids Research 14:4683- 4690; and Nielsen, et al. (1997) Protein Eng. 10: 1-12, and the precise amino acid composition of the signal peptide often does not appear to be critical to its function, e.g., Randall, et al. (1989) Science 243:1156-1159; and Kaiser, et al. (1987) Science 235:312-317. The mature proteins of the invention can be readily determined using standard methods.

It will often be desired to express these polypeptides in a system which provides a specific or defined glycosylation pattern. In this case, the usual pattern will be that provided naturally by the expression system. However, the pattern will be modifiable by exposing the polypeptide, e.g., an unglycosylated form, to appropriate glycosylating proteins introduced into a heterologous expression system. For example, the receptor gene may be co-transformed with one or more genes encoding mammalian or other glycosylating enzymes. Using this approach, certain mammalian glycosylation patterns will be achievable in prokaryote or other cells. Expression in prokaryote cells will typically lead to unglycosylated forms of protein.

The source of DCRS8 can be a eukaryotic or prokaryotic host expressing recombinant DCRS8, such as is described above. The source can also be a cell line, but other mammalian cell lines are also contemplated by this invention, with the preferred cell line being from the human species.

Now that the sequences are known, the primate DCRS8 or DCRS9, fragments, or derivatives thereof can be prepared by conventional processes for synthesizing peptides. These include processes such as are described in Stewart and Young (1984) Solid Phase Peptide Synthesis, Pierce Chemical Co., Rockford, IL; Bodanszky and Bodanszky (1984) The Practice of Peptide Synthesis, Springer-Verlag, New York; and Bodanszky (1984) The Principles of Peptide Synthesis, Springer-Verlag, New York; all of each which are incorporated herein by reference. For example, an azide process, an acid chloride process, an acid anhydride process, a mixed anhydride process, an active ester process (for example, p-nitrophenyl ester, N-hydroxysuccinimide ester, or cyanomethyl ester), a carbodiimidazole process, an oxidative-reductive process, or a dicyclohexylcarbodiimide (DCCD)/additive process can be used. Solid phase and solution phase syntheses are both applicable to the foregoing processes. Similar techniques can be used with partial DCRS8 or DCRS9 sequences.

The DCRS8 proteins, fragments, or derivatives are suitably prepared in accordance with the above processes as typically employed in peptide synthesis, generally either by a so-called stepwise process which comprises condensing an amino acid to the terminal amino acid, one by one in sequence, or by coupling peptide fragments to the terminal amino acid. Amino groups that are not being used in the coupling reaction typically must be protected to prevent coupling at an incorrect location.

If a solid phase synthesis is adopted, the C-terminal amino acid is bound to an insoluble carrier or support through its carboxyl group. The insoluble carrier is not particularly limited as long as it has a binding capability to a reactive carboxyl group. Examples of such insoluble carriers include halomethyl resins, such as chloromethyl resin or bromomethyl resin, hydroxymethyl resins, phenol resins, tert-alkyloxycarbonylhydrazidated resins, and the like.

An amino group-protected amino acid is bound in sequence through condensation of its activated carboxyl group and the reactive amino group of the previously formed peptide or chain, to synthesize the peptide step by step. After synthesizing the complete sequence, the peptide is split off from the insoluble carrier to produce the peptide. This solid-phase approach is generally described by Merrifield, et al. (1963) in J. Am. Chem. Soc. 85:2149-2156, which is incorporated herein by reference.

The prepared protein and fragments thereof can be isolated and purified from the reaction mixture by means of peptide separation, e.g., by extraction, precipitation, electrophoresis, various forms of chromatography, and the like. The receptors of this invention can be obtained in varying degrees of purity depending upon desired uses. Purification can be accomplished by use of the protein purification techniques disclosed herein, see below, or by the use of the antibodies herein described in methods of immunoabsorbant affinity chromatography. This immunoabsorbant affinity chromatography is carried out by first linking the antibodies to a solid support and then contacting the linked antibodies with solubilized lysates of appropriate cells, lysates of other cells expressing the receptor, or lysates or supernatants of cells producing the protein as a result of DNA techniques, see below.

Generally, the purified protein will be at least about 40% pure, ordinarily at least about 50% pure, usually at least about 60% pure, typically at least about 70% pure, more typically at least about 80% pure, preferable at least about 90% pure and more preferably at least about 95% pure, and in particular embodiments, 97%-99% or more. Purity will usually be on a weight basis, but can also be on a molar basis. Different assays will be applied as appropriate. Individual proteins may be purified and thereafter combined.

### VI. Antibodies

Antibodies can be raised to the various mammalian, e.g., primate DCRS8 or DCRS9 proteins and fragments thereof, both in naturally occurring native forms and in their recombinant forms, the difference being that antibodies to the active receptor are more likely to recognize epitopes which are only present in the native conformations. Denatured antigen detection can also be useful in, e.g., Western analysis. Anti-idiotypic antibodies are also contemplated, which would be useful as agonists or antagonists of a natural receptor or an antibody.

Antibodies, including binding fragments and single chain versions, against predetermined fragments of the protein can be raised by immunization of animals with conjugates of the fragments with immunogenic proteins. Monoclonal antibodies are prepared from cells secreting the desired antibody. These antibodies can be screened for binding to normal or defective protein, or screened for agonistic or antagonistic activity. These monoclonal antibodies will usually bind with at least a KD of about 1 mM, more usually at least about 300 μM, typically at least about 100μM, more typically at least about 30 gM, preferably at least about 10 μM, and more preferably at least about 3 JM or better.

The antibodies, including antigen binding fragments, of this invention can have significant diagnostic or therapeutic value. They can be potent antagonists that bind to the receptor and inhibit binding to ligand or inhibit the ability of the receptor to elicit a biological response, e.g., act on its substrate. They also can be useful as non-neutralizing antibodies and can be coupled to toxins or radionuclides to bind producing cells, or cells localized to the source of the interleukin. Further, these antibodies can be conjugated to drugs or other therapeutic agents, either directly or indirectly by means of a linker.

The antibodies of this invention can also be useful in diagnostic applications. As capture or non-neutralizing antibodies, they might bind to the receptor without inhibiting ligand or substrate binding. As neutralizing antibodies, they can be useful in competitive binding assays. They will also be useful in detecting or quantifying ligand. They may be used as reagents for Western blot analysis, or for immunoprecipitation or immunopurification of the respective protein. Likewise, nucleic acids and proteins may be immobilized to solid substrates for affinity purification or detection methods. The substrates may be, e.g., solid resin beads or sheets of plastic.

Protein fragments may be joined to other materials, particularly polypeptides, as fused or covalently joined polypeptides to be used as immunogens. Mammalian cytokine receptors and fragments may be fused or covalently linked to a variety of immunogens, such as keyhole limpet hemocyanin, bovine serum albumin, tetanus toxoid, etc. See (1969) Microbiology, Hoeber Medical Division, Harper and Row; Landsteiner (1962) Specificity of Serological Reactions, Dover Publications, N.Y.; and Williams, et al. (1967) Methods in lmnunology and Immunochemistry, Vol. 1, Academic Press, N.Y.; each of which is incorporated herein by reference, for descriptions of methods of preparing polyclonal antisera. A typical method involves hyperimmunization of an animal with an antigen. The blood of the animal is then collected shortly after the repeated immunizations and the gamma globulin is isolated.

In some instances, it is desirable to prepare monoclonal antibodies from various mammalian hosts, such as mice, rodents, primates, humans, etc. Description of techniques for preparing such monoclonal antibodies may be found in, e.g., Stites, et al. (eds.) Basic and Clinical Immunology (4th ed.), Lange Medical Publications, Los Altos, CA, and references cited therein; Harlow and Lane (1988) Antibodies: A Laboratorv Manual, CSH Press; Goding (1986) Monoclonal Antibodies: Principles and Practice (2d ed.) Academic Press, N.Y.; and particularly in Kohler and Milstein (1975) Nature 256:495-497, which discusses one method of generating monoclonal antibodies. Each of these references is incorporated herein by reference. Summarized briefly, this method involves injecting an animal with an immunogen. The animal is then sacrificed and cells taken from its spleen, which are then fused with myeloma cells. The result is a hybrid cell or “hybridoma” that is capable of reproducing in vitro. The population of hybridomas is then screened to isolate individual clones, each of which secrete a single antibody species to the immunogen. In this manner, the individual antibody species obtained are the products of immortalized and cloned single B cells from the immune animal generated in response to a specific site recognized on the immunogenic substance.

Other suitable techniques involve in vitro exposure of lymphocytes to the antigenic polypeptides or alternatively to selection of libraries of antibodies in phage or similar vectors. See, Huse, et al. (1989) “Generation of a Large Combinatorial Library of the Immunoglobulin Repertoire in Phage Lambda,” Science 246:1275-1281; and Ward, et al. (1989) Nature 341:544-546, each of which is incorporated herein by reference. The polypeptides and antibodies of the present invention may be used with or without modification, including chimeric or humanized antibodies. Frequently, the polypeptides and antibodies will be labeled by joining, either covalently or non-covalently, a substance which provides for a detectable signal. A wide variety of labels and conjugation techniques are known and are reported extensively in both the scientific and patent literature. Suitable labels include radionuclides, enzymes, substrates, cofactors, inhibitors, fluorescent moieties, chemiluminescent moieties, magnetic particles, and the like. Patents, teaching the use of such labels include U.S. Patent Nos. 3,817,837; 3,850,752; 3,939,350; 3,996,345; 4,277,437; 4,275,149; and 4,366,241. Also, recombinant or chimeric immunoglobulins may be produced, see Cabilly, U.S. Patent No. 4,816,567; or made in transgenic mice, see Mendez, et al. (1997) Nature Genetics 15:146- 156; Abgenix; and Medarex. These references are incorporated herein by reference.

The antibodies of this invention can also be used for affinity chromatography in isolating the DCRS8 proteins or peptides. Columns can be prepared where the antibodies are linked to a solid support, e.g., particles, such as agarose, Sephadex, or the like, where a cell lysate may be passed through the column, the column washed, followed by increasing concentrations of a mild denaturant, whereby the purified protein will be released. Alternatively, the protein may be used to purify antibody. Appropriate cross absorptions or depletions may be applied.

The antibodies may also be used to screen expression libraries for particular expression products. Usually the antibodies used in such a procedure will be labeled with a moiety allowing easy detection of presence of antigen by antibody binding.

Antibodies raised against a cytokine receptor will also be used to raise anti- idiotypic antibodies. These will be useful in detecting or diagnosing various immunological conditions related to expression of the protein or cells which express the protein. They also will be useful as agonists or antagonists of the ligand, which may be competitive inhibitors or substitutes for naturally occurring ligands.

A cytokine receptor protein that specifically binds to or that is specifically immunoreactive with an antibody generated against a defined immunogen, such as an immunogen consisting of the amino acid sequence of SEQ ID NO: 14, is typically determined in an immunoassay. The immunoassay typically uses a polyclonal antiserum which was raised, e.g., to a protein of SEQ ID NO: 14. This antiserum is selected to have low crossreactivity against other cytokine receptor family members, preferably from the same species, and any such crossreactivity is removed by immunoabsorption prior to use in the immunoassay.

In order to produce antisera for use in an immunoassay, the protein, e.g., of SEQ ID NO: 14, is isolated as described herein. For example, recombinant protein may be produced in a mammalian cell line. An appropriate host, e.g., an inbred strain of mice such as Balb/c, is immunized with the selected protein, typically using a standard adjuvant, such as Freund's adjuvant, and a standard mouse immunization protocol (see Harlow and Lane, supra). Alternatively, a synthetic peptide derived from the sequences disclosed herein and conjugated to a carrier protein can be used an immunogen. Polyclonal sera are collected and titered against the immunogen protein in an immunoassay, e.g., a solid phase immunoassay with the immunogen immobilized on a solid support. Polyclonal antisera with a titer of 104 or greater are selected and tested for their cross reactivity against other cytokine receptor family members using a competitive binding immunoassay such as the one described in Harlow and Lane, supra, at pages 570- 573. Preferably at least two cytokine receptor family members are used in this determination. These cytokine receptor family members can be produced as recombinant proteins and isolated using standard molecular biology and protein chemistry techniques as described herein.

Immunoassays in the competitive binding format can be used for the crossreactivity determinations. For example, the protein of SEQ ID NO: 14 can be immobilized to a solid support. Proteins added to the assay compete with the binding of the antisera to the immobilized antigen. The ability of the above proteins to compete with the binding of the antisera to the immobilized protein is compared to the other proteins. The percent crossreactivity for the above proteins is calculated, using standard calculations. Those antisera with less than 10% crossreactivity with each of the proteins listed above are selected and pooled. The cross-reacting antibodies are then removed from the pooled antisera by immunoabsorption with the above-listed proteins.

The immunoabsorbed and pooled antisera are then used in a competitive binding immunoassay as described above to compare a second protein to the immunogen protein (e.g., the DCRS8 like protein of SEQ ID NO: 14). In order to make this comparison, the two proteins are each assayed at a wide range of concentrations and the amount of each protein required to inhibit 50% of the binding of the antisera to the immobilized protein is determined. If the amount of the second protein required is less than twice the amount of the protein of the selected protein or proteins that is required, then the second protein is said to specifically bind to an antibody generated to the immunogen.

It is understood that these cytokine receptor proteins are members of a family of homologous proteins that comprise at least 9 so far identified members, 6 mammalian and 3 worm embodiments. For a particular gene product, such as the DCRS8, the term refers not only to the amino acid sequences disclosed herein, but also to other proteins that are allelic, non-allelic, or species variants. It is also understood that the terms include nonnatural mutations introduced by deliberate mutation using conventional recombinant technology such as single site mutation, or by excising short sections of DNA encoding the respective proteins, or by substituting new amino acids, or adding new amino acids. Such minor alterations typically will substantially maintain the immunoidentity of the original molecule and/or its biological activity. Thus, these alterations include proteins that are specifically immunoreactive with a designated naturally occurring DCRS8 protein. The biological properties of the altered proteins can be determined by expressing the protein in an appropriate cell line and measuring the appropriate effect, e.g., upon transfected lymphocytes. Particular protein modifications considered minor would include conservative substitution of amino acids with similar chemical properties, as described above for the cytokine receptor family as a whole. By aligning a protein optimally with the protein of the cytokine receptors and by using the conventional immunoassays described herein to determine immunoidentity, one can determine the protein compositions of the invention.

### VII. Kits and quantitation

Both naturally occurring and recombinant forms of the cytokine receptor like molecules of this invention are particularly useful in kits and assay methods. For example, these methods would also be applied to screening for binding activity, e.g., ligands for these proteins. Several methods of automating assays have been developed in recent years so as to permit screening of tens of thousands of compounds per year. See, e.g., a BIOMEK automated workstation, Beckman Instruments, Palo Alto, California, and Fodor, et al. (1991) Science 251:767-773, which is incorporated herein by reference. The latter describes means for testing binding by a plurality of defined polymers synthesized on a solid substrate. The development of suitable assays to screen for a ligand or agonist/antagonist homologous proteins can be greatly facilitated by the availability of large amounts of purified, soluble cytokine receptors in an active state such as is provided by this invention.

Purified protein can be coated directly onto plates for use in the aforementioned ligand screening techniques. However, non-neutralizing antibodies to these proteins can be used as capture antibodies to immobilize the respective receptor on the solid phase, useful, e.g., in diagnostic uses.

This invention also contemplates use of receptor subunit, fragments thereof, peptides, and their fusion products in a variety of diagnostic kits and methods for detecting the presence of the protein or its ligand. Alternatively, or additionally, antibodies against the molecules may be incorporated into the kits and methods. Typically the kit will have a compartment containing, e.g., a DCRS8 peptide or gene segment or a reagent which recognizes one or the other. Typically, recognition reagents, in the case of peptide, would be a receptor or antibody, or in the case of a gene segment, would usually be a hybridization probe.

A preferred kit for determining the concentration of DCRS8 in a sample would typically comprise a labeled compound, e.g., ligand or antibody, having known binding affinity for DCRS8, a source of DCRS8 (naturally occurring or recombinant) as a positive control, and a means for separating the bound from free labeled compound, e.g., a solid phase for immobilizing the DCRS8 in the test sample. Compartments containing reagents, and instructions, will normally be provided. Appropriate nucleic acid or protein containing kits are also provided.

Antibodies, including antigen binding fragments, specific for mammalian DCRS8 or a peptide fragment, or receptor fragments are useful in diagnostic applications to detect the presence of elevated levels of ligand and/or its fragments. Diagnostic assays may be homogeneous (without a separation step between free reagent and antibody-antigen complex) or heterogeneous (with a separation step). Various commercial assays exist, such as radioimmunoassay (RIA), enzyme-linked immunosorbent assay (ELISA), enzyme immunoassay (EIA), enzyme-multiplied immunoassay technique (EMIT), substrate-labeled fluorescent immunoassay (SLFIA) and the like. For example, unlabeled antibodies can be employed by using a second antibody which is labeled and which recognizes the antibody to a cytokine receptor or to a particular fragment thereof. These assays have also been extensively discussed in the literature. See, e.g., Harlow and Lane (1988) Antibodies: A Laboratory Manual, CSH, and Coligan (ed. 1991 and periodic supplements) Current Protocols In Immunology Greene/Wiley, N.Y.

Anti-idiotypic antibodies may have similar use to serve as agonists or antagonists of cytokine receptors. These should be useful as therapeutic reagents under appropriate circumstances.

Frequently, the reagents for diagnostic assays are supplied in kits, so as to optimize the sensitivity of the assay. For the subject invention, depending upon the nature of the assay, the protocol, and the label, either labeled or unlabeled antibody, or labeled ligand is provided. This is usually in conjunction with other additives, such as buffers, stabilizers, materials necessary for signal production such as substrates for enzymes, and the like. Preferably, the kit will also contain instructions for proper use and disposal of the contents after use. Typically the kit has compartments for each useful reagent, and will contain instructions for proper use and disposal of reagents. Desirably, the reagents are provided as a dry lyophilized powder, where the reagents may be reconstituted in an aqueous medium having appropriate concentrations for performing the assay.

The aforementioned constituents of the diagnostic assays may be used without modification or may be modified in a variety of ways. For example, labeling may be achieved by covalently or non-covalently joining a moiety which directly or indirectly provides a detectable signal. In many of these assays, a test compound, cytokine receptor, or antibodies thereto can be labeled either directly or indirectly. Possibilities for direct labeling include label groups: radiolabels such as 1251, enzymes (U.S. Pat. No. 3,645,090) such as peroxidase and alkaline phosphatase, and fluorescent labels (U.S. Pat. No. 3,940,475) capable of monitoring the change in fluorescence intensity, wavelength shift, or fluorescence polarization. Both of the patents are incorporated herein by reference. Possibilities for indirect labeling include biotinylation of one constituent followed by binding to avidin coupled to one of the above label groups.

There are also numerous methods of separating the bound from the free ligand, or alternatively the bound from the free test compound. The cytokine receptor can be immobilized on various matrixes followed by washing. Suitable matrices include plastic such as an ELISA plate, filters, and beads. Methods of immobilizing the receptor to a matrix include, without limitation, direct adhesion to plastic, use of a capture antibody, chemical coupling, and biotin-avidin. The last step in this approach involves the precipitation of antibody/antigen complex by any of several methods including those utilizing, e.g., an organic solvent such as polyethylene glycol or a salt such as ammonium sulfate. Other suitable separation techniques include, without limitation, the fluorescein antibody magnetizable particle method described in Rattle, et al. (1984) Clin. Chem. 30(9):1457-1461, and the double antibody magnetic particle separation as described in U.S. Pat. No. 4,659,678, each of which is incorporated herein by reference.

The methods for linking protein or fragments to various labels have been extensively reported in the literature and do not require detailed discussion here. Many of the techniques involve the use of activated carboxyl groups either through the use of carbodiimide or active esters to form peptide bonds, the formation of thioethers by reaction of a mercapto group with an activated halogen such as chloroacetyl, or an activated olefin such as maleimide, for linkage, or the like. Fusion proteins will also find use in these applications.

Another diagnostic aspect of this invention involves use of oligonucleotide or polynucleotide sequences taken from the sequence of an cytokine receptor. These sequences can be used as probes for detecting levels of the respective cytokine receptor in patients suspected of having an immunological disorder. The preparation of both RNA and DNA nucleotide sequences, the labeling of the sequences, and the preferred size of the sequences has received ample description and discussion in the literature. Normally an oligonucleotide probe should have at least about 14 nucleotides, usually at least about 18 nucleotides, and the polynucleotide probes may be up to several kilobases. Various labels may be employed, most commonly radionuclides, particularly 32p However, other techniques may also be employed, such as using biotin modified nucleotides for introduction into a polynucleotide. The biotin then serves as the site for binding to avidin or antibodies, which may be labeled with a wide variety of labels, such as radionuclides, fluorescers, enzymes, or the like. Alternatively, antibodies may be employed which can recognize specific duplexes, including DNA duplexes, RNA duplexes, DNA-RNA hybrid duplexes, or DNA-protein duplexes. The antibodies in turn may be labeled and the assay carried out where the duplex is bound to a surface, so that upon the formation of duplex on the surface, the presence of antibody bound to the duplex can be detected. The use of probes to the novel RNA may be carried out in conventional techniques such as nucleic acid hybridization, plus and minus screening, recombinational probing, hybrid released translation (HRT), and hybrid arrested translation (HART). Antisense nucleic acids, which may be used to block protein expression, are also provided. See, e.g., Isis Pharmaceuticals, Sequitur, Inc., or Hybridon. This also includes amplification techniques such as polymerase chain reaction (PCR).

Diagnostic kits which also test for the qualitative or quantitative presence of other markers are also contemplated. Diagnosis or prognosis may depend on the combination of multiple indications used as markers. Thus, kits may test for combinations of markers. See, e.g., Viallet, et al. (1989) Progress in Growth Factor Res. 1:89-97.

### VIII. Therapeutic Utility This invention provides reagents with significant therapeutic value. See, e.g., Levitzki (1996) Curr. Opin. Cell Biol. 8:239-244. The cytokine receptors (naturally occurring or recombinant), fragments thereof, mutein receptors, and antibodies, along with compounds identified as having binding affinity to the receptors or antibodies, should be useful in the treatment of conditions exhibiting abnormal expression of the receptors of their ligands. Such abnormality will typically be manifested by immunological disorders, e.g., innate immunity, or developmentally. Additionally, this invention should provide therapeutic value in various diseases or disorders associated with abnormal expression or abnormal triggering of response to the ligand. For example, the IL-1 ligands have been suggested to be involved in morphologic development, e.g., dorso-ventral polarity determination, and immune responses, particularly the primitive innate responses. See, e.g., Sun, et al. (1991) Eur. J. Biochem. 196:247-254; and Hultruark (1994) Nature 367:116-117.

Recombinant cytokine receptors, muteins, agonist or antagonist antibodies thereto, or antibodies can be purified and then administered to a patient. These reagents can be combined for therapeutic use with additional active ingredients, e.g., in conventional pharmaceutically acceptable carriers or diluents, along with physiologically innocuous stabilizers and excipients. These combinations can be sterile, e.g., filtered, and placed into dosage forms as by lyophilization in dosage vials or storage in stabilized aqueous preparations. This invention also contemplates use of antibodies or binding fragments thereof which are not complement binding.

Ligand screening using cytokine receptor or fragments thereof can be performed to identify molecules having binding affinity to the receptors. Subsequent biological assays can then be utilized to determine if a putative ligand can provide competitive binding, which can block intrinsic stimulating activity. Receptor fragments can be used as a blocker or antagonist in that it blocks the activity of ligand. Likewise, a compound having intrinsic stimulating activity can activate the receptor and is thus an agonist in that it simulates the activity of ligand, e.g., inducing signaling. This invention further contemplates the therapeutic use of antibodies to cytokine receptors as antagonists.

The quantities of reagents necessary for effective therapy will depend upon many different factors, including means of administration, target site, reagent physiological life, pharmacological life, physiological state of the patient, and other medicants administered. Thus, treatment dosages should be titrated to optimize safety and efficacy. Typically, dosages used in vitro may provide useful guidance in the amounts useful for in situ administration of these reagents. Animal testing of effective doses for treatment of particular disorders will provide further predictive indication of human dosage. Various considerations are described, e.g., in Gilman, et al. (eds. 1990) Goodman and Gihnan's: The Pharmacological Bases of Therapeutics, 8th Ed., Pergamon Press; and Remington's Pharmaceutical Sciences, 17th ed. (1990), Mack Publishing Co., Easton, Penn.; each of which is hereby incorporated herein by reference. Methods for administration are discussed therein and below, e.g., for oral, intravenous, intraperitoneal, or intramuscular administration, transdermal diffusion, and others. Pharmaceutically acceptable carriers will include water, saline, buffers, and other compounds described, e.g., in the Merck Index, Merck & Co., Rahway, New Jersey. Because of the likely high affinity binding, or turnover numbers, between a putative ligand and its receptors, low dosages of these reagents would be initially expected to be effective. And the signaling pathway suggests extremely low amounts of ligand may have effect. Thus, dosage ranges would ordinarily be expected to be in amounts lower than 1 mM concentrations, typically less than about 10 [LM concentrations, usually less than about 100 nM, preferably less than about 10 pM (picomolar), and most preferably less than about 1 fIM (femtomolar), with an appropriate carrier. Slow release formulations, or slow release apparatus will often be utilized for continuous administration.

Cytokine receptors, fragments thereof, and antibodies or its fragments, antagonists, and agonists, may be administered directly to the host to be treated or, depending on the size of the compounds, it may be desirable to conjugate them to carrier proteins such as ovalbumin or serum albumin prior to their administration. Therapeutic formulations may be administered in many conventional dosage formulations. While it is possible for the active ingredient to be administered alone, it is preferable to present it as a pharmaceutical formulation. Formulations comprise at least one active ingredient, as defined above, together with one or more acceptable carriers thereof. Each carrier must be both pharmaceutically and physiologically acceptable in the sense of being compatible with the other ingredients and not injurious to the patient. Formulations include those suitable for oral, rectal, nasal, or parenteral (including subcutaneous, intramuscular, intravenous and intradermal) administration. The formulations may conveniently be presented in unit dosage form and may be prepared by methods well known in the art of pharmacy. See, e.g., Gilman, et al. (eds. 1990) Goodman and Gilman's: The Pharmacological Bases of Therapeutics, 8th Ed., Pergamon Press; and Remington's Pharmaceutical Sciences, 17th ed. (1990), Mack Publishing Co., Easton, Penn.; Avis, et al. (eds. 1993) Pharmaceutical Dosage Forms: Parenteral Medications Dekker, N.Y.; Lieberman, et al. (eds. 1990) Pharmaceutical Dosage Forms: Tablets Dekker, N.Y.; and Lieberman, et al. (eds. 1990) Pharmaceutical Dosage Forms: Disperse Systems Dekker, N.Y. The therapy of this invention may be combined with or used in association with other therapeutic agents, particularly agonists or antagonists of other cytokine receptor family members.

### IX. Screening

Drug screening using DCRS8 or fragments thereof can be performed to identify compounds having binding affinity to the receptor subunit, including isolation of associated components. Subsequent biological assays can then be utilized to determine if the compound has intrinsic stimulating activity and is therefore a blocker or antagonist in that it blocks the activity of the ligand. Likewise, a compound having intrinsic stimulating activity can activate the receptor and is thus an agonist in that it simulates the activity of a cytokine ligand. This invention further contemplates the therapeutic use of antibodies to the receptor as cytokine agonists or antagonists.

Similarly, complexes comprising multiple proteins may be used to screen for ligands or reagents capable of recognizing the complex. Most cytokine receptors comprise at least two subunits, which may be the same, or distinct. Alternatively, the transmembrane receptor may bind to a complex comprising a cytokine-like ligand associated with another soluble protein serving, e.g., as a second receptor subunit.

One method of drug screening utilizes eukaryotic or prokaryotic host cells which are stably transformed with recombinant DNA molecules expressing the DCRS8 in combination with another cytokine receptor subunit. Cells may be isolated which express a receptor in isolation from other finctional receptors. Such cells, either in viable or fixed form, can be used for standard antibody/antigen or ligand/receptor binding assays. See also, Parce, et al. (1989) Science 246:243-247; and Owicki, et al. (1990) Proc. Nat'l Acad. Sci. USA 87:4007-4011, which describe sensitive methods to detect cellular responses. Competitive assays are particularly useful, where the cells (source of putative ligand) are contacted and incubated with a labeled receptor or antibody having known binding affinity to the ligand, such as ¹²⁵I-antibody, and a test sample whose binding affinity to the binding composition is being measured. The bound and free labeled binding compositions are then separated to assess the degree of ligand binding. The amount of test compound bound is inversely proportional to the amount of labeled receptor binding to the known source. Many techniques can be used to separate bound from free ligand to assess the degree of ligand binding. This separation step could typically involve a procedure such as adhesion to filters followed by washing, adhesion to plastic followed by washing, or centrifugation of the cell membranes. Viable cells could also be used to screen for the effects of drugs on cytokine mediated functions, e.g., second messenger levels, e.g., Ca++; cell proliferation; inositol phosphate pool changes; and others. Some detection methods allow for elimination of a separation step, e.g., a proximity sensitive detection system. Calcium sensitive dyes will be useful for detecting Ca++levels, with a fluorimeter or a fluorescence cell sorting apparatus.

### X. Ligands

The descriptions of the DCRS8 herein provides means to identify ligands, as described above. Such ligand should bind specifically to the respective receptor with reasonably high affinity. Various constructs are made available which allow either labeling of the receptor to detect its ligand. For example, directly labeling cytokine receptor, fuising onto it markers for secondary labeling, e.g., FLAG or other epitope tags, etc., will allow detection of receptor. This can be histological, as an affinity method for biochemical purification, or labeling or selection in an expression cloning approach. A two-hybrid selection system may also be applied making appropriate constructs with the available cytokine receptor sequences. See, e.g., Fields and Song (1989) Nature 340:245- 246.

Most likely candidates will be structually related to members of the IL-1 7 family. See, e.g., USSN 09/480,287.

The broad scope of this invention is best understood with reference to the following examples, which are not intended to limit the inventions to the specific embodiments.

### EXAMPLES

### I. General Methods

Some of the standard methods are described or referenced, e.g., in Maniatis, et al. (1982) Molecular Cloning. A Laboratory Manual, Cold Spring Harbor Laboratory, Cold Spring Harbor Press; Sambrook, et al. (1989) Molecular Cloning: A Laboratory Manual, (2d ed.), vols. 1-3, CSH Press, N.Y.; or Ausubel, et al. (1987 and Supplements) Current Protocols in Molecular Biology, Greene/Wiley, N.Y. Methods for protein purification include such methods as ammonium sulfate precipitation, column chromatography, electrophoresis, centrifugation, crystallization, and others. See, e.g., Ausubel, et al. (1987 and periodic supplements); Coligan, et al. (ed. 1996) and periodic supplements, Current Protocols In Protein Science Greene/Wiley, N.Y.; Deutscher (1990) “Guide to Protein Purification” in Methods in Enzymology, vol. 182, and other volumes in this series; and manufacturer's literature on use of protein purification products, e.g., Pharmacia, Piscataway, N.J., or Bio-Rad, Richmond, CA. Combination with recombinant techniques allow fusion to appropriate segments, e.g., to a FLAG sequence or an equivalent which can be fused via a protease-removable sequence. See, e.g., Hochuli (1990) “Purification of Recombinant Proteins with Metal Chelate Absorbent” in Setlow (ed.) Genetic Engineering. Principle and Methods 12:87-98, Plenum Press, N.Y.; and Crowe, et al. (1992) QIAexpress: The High Level Expression & Protein Purification Svstem QUIAGEN, Inc., Chatsworth, Calif.

Computer sequence analysis is performed, e.g., using available software programs, including those from the GCG (U. Wisconsin) and GenBank sources. Public sequence databases were also used, e.g., from GenBank and others.

Many techniques applicable to IL-10 receptors may be applied to the DCRSs, as described, e.g., in USSN 08/110,683 (IL-10 receptor), which is incorporated herein by reference.

### II . Computational Analysis

Human sequences related to cytokine receptors were identified from genomic sequence database using, e.g., the BLAST server (Altschul, et al. (1994) Nature Genet. 6:119-129). Standard analysis programs may be used to evaluate structure, e.g., PHD (Rost and Sander (1994) Proteins 19:55-72) and DSC (King and Stemberg (1996) Protein Sci. 5:2298-23 10). Standard comparison software includes, e.g., Altschul, et al. (I1990) J. Mol. Biol. 215:403-10; Waterman (1995) Introduction to Computational Biologv: Maps, Sequences, and Genomes Chapman & Hall; Lander and Watermnan (eds. 1995) Calculating the Secrets of Life: App2lications of the Mathematical Sciences in Molecular Biology National Academy Press; and Speed and Waterman (eds. 1996) Genetic Mapping and DNA Sequencing (IMA Volumes in Mathematics and Its Applications, Vol 8 1) Springer Verlag. Each reference is incorporate herein by reference.

### III. Cloning of fall-length cDNAs; Chromosomal localization

PCR primers derived from the sequences are used to probe a human cDNA library. Sequences may be derived, e.g., from Tables 1-5, preferably those adjacent the ends of sequences. Full length cDNAs for primate, rodent, or other species DCRS8 are cloned, e.g., by DNA hybridization screening of kgtlO phage. PCR reactions are conducted using T. aquaticus Taqplus DNA polymerase (Stratagene) under appropriate conditions. Extending partial length cDNA clones is typically routine.

Chromosome spreads are prepared. In situ hybridization is performed on chromosome preparations obtained from phytohemagglutinin-stimulated human lymphocytes cultured for 72 h. 5-bromodeoxynridine was added for the final seven hours of culture (60 [tg/ml of medium), to ensure a posthybridization chromosomal banding of good quality.

A PCR fragment, amplified with the help of primers, is cloned into an appropriate vector. The vector is labeled by nick-translation with ³H. The radiolabeled probe is hybridized to metaphase spreads at final concentration of 200 ng/ml of hybridization solution as described, e.g., in Mattei, et al. (1985) Hum. Genet. 69:327-331.

After coating with nuclear track emulsion (KODAK NTB2), slides are exposed. To avoid any slipping of silver grains during the banding procedure, chromosome spreads are first stained with buffered Giemsa solution and metaphase photographed. R-banding is then performed by the fluorochrome-photolysis-Giemsa (FPG) method and metaphases rephotographed before analysis.

Similar appropriate methods are used for other species. IV. Localization of MRNA Human multiple tissue (Cat# 1, 2) and cancer cell line blots (Cat# 7757-1), containing approximately 2 tg of poly(A)+RNA per lane, are purchased from Clontech (Palo Alto, CA). Probes are radiolabeled with [α-³²p] dATP, e.g., using the Amersham Rediprime random primer labeling kit (RPN1633). Prehybridization and hybridizations are performed, e.g., at 650 C in 0.5 M Na2HPO4, 7% SDS, 0.5 M EDTA (pH 8.0). High stringency washes are conducted, e.g., at 65° C with two initial washes in 2 x SSC, 0.1% SDS for 40 min followed by a subsequent wash in 0.1 x SSC, 0.1% SDS for 20 min. Membranes are then exposed at -70° C to X-Ray film (Kodak) in the presence of intensifying screens. More detailed studies by cDNA library Southerns are performed with selected appropriate human DCRS clones to examine their expression in hemopoietic or other cell subsets.

Alternatively, two appropriate primers are selected from Tables 1-5. RT-PCR is used on an appropriate mRNA sample selected for the presence of message to produce a cDNA, e.g., a sample which expresses the gene.

Full length clones may be isolated by hybridization of cDNA libraries from appropriate tissues pre-selected by PCR signal. Northern blots can be performed.

Message for genes encoding DCRS will be assayed by appropriate technology, e.g., PCR, immunoassay, hybridization, or otherwise. Tissue and organ cDNA preparations are available, e.g., from Clontech, Mountain View, CA. Identification of sources of natural expression are useful, as described. And the identification of functional receptor subunit pairings will allow for prediction of what cells express the combination of receptor subunits which will result in a physiological responsiveness to each of the cytokine ligands.

For mouse counterpart distribution, e.g., Southern Analysis can be performed: DNA (5 pg) from a primary amplified cDNA library was digested with appropriate restriction enzymes to release the inserts, run on a 1% agarose gel and transferred to a nylon membrane (Schleicher and Schuell, Keene, NH).

Samples for mouse MRNA isolation may include: resting mouse fibroblastic L cell line (C200); Braf:ER (Braf fusion to estrogen receptor) transfected cells, control (C201); T cells, TH1 polarized (Mell4 bright, CD4+cells from spleen, polarized for 7 days with IFN-y and anti IL-4; T200); T cells, TH2 polarized (Mell4 bright, CD4+cells from spleen, polarized for 7 days with IL-4 and anti-IFN-y; T201); T cells, highly THI polarized (see Openshaw, et al. (1995) J. Exp. Med. 182:1357-1367; activated with anti-CD3 for 2, 6, 16 h pooled; T202); T cells, highly TH2 polarized (see Openshaw, et al. (1995) J. Exp. Med. 182:1357-1367; activated with anti-CD3 for 2, 6, 16 h pooled; T203); CD44- CD25+pre T cells, sorted from thymus (T204); TH1 T cell clone Dl.1, resting for 3 weeks after last stimulation with antigen (T205); TH1 T cell clone Dl. 1, 10 ,ug/ml ConA stimulated 15 h (T206); TH2 T cell clone CDC35, resting for 3 weeks after last stimulation with antigen (T207); TH2 T cell clone CDC35, 10 tg/ml ConA stimulated 15 h (T208); Mell4+naive T cells from spleen, resting (T209); Mell4+T cells, polarized to Thl with IFN-y/IL-12/anti-IL-4 for 6, 12, 24 h pooled (T210); Mell4+T cells, polarized to Th2 with IL-4/anti-IFN-y for 6, 13, 24 h pooled (T21 1); unstimulated mature B cell leukemia cell line A20 (B200); unstimulated B cell line CH12 (B201); unstimulated large B cells from spleen (B202); B cells from total spleen, LPS activated (B203); metrizamide enriched dendritic cells from spleen, resting (D200); dendritic cells from bone marrow, resting (D201); monocyte cell line RAW 264.7 activated with LPS 4 h (M200); bone-marrow macrophages derived with GM and M-CSF (M201); macrophage cell line J774, resting (M202); macrophage cell line J774 +LPS +anti-IL-10 at 0.5, 1, 3, 6, 12 h pooled (M203); macrophage cell line J774 +LPS +IL-10 at 0.5, 1, 3, 5, 12 h pooled(M204); aerosol challenged mouse lung tissue, Th2 primers, aerosol OVA challenge 7, 14, 23 h pooled (see Garlisi, et al. (1995) Clinical Immunology and immunopathologv 75:75-83; X206); Nippostrongulus-infected lung tissue (see Coffmnan, et al. (1989) Science 245:308-310; X200); total adult lung, normal (O200); total lung, rag-i (see Schwarz, et al. (1993) Immunodeficiency 4:249-252; 0205); IL-10 K.O. spleen (see Kuhn, et al. (1991) Cell 75:263-274; X201); total adult spleen, normal (0201); total spleen, rag-i (0207); IL-10 K.O. Peyer's patches (0202); total Peyer's patches, normal (0210); IL-10 K.O. mesenteric lymph nodes (X203); total mesenteric lymph nodes, normal (0211); IL-10 K.O. colon (X203); total colon, normal (0212); NOD mouse pancreas (see Makino, et al. (1980) Jikken Dobutsu 29:1-13; X205); total thymus, rag-i (0208); total kidney, rag-l (0209); total heart, rag-i (0202); total brain, rag-i (0203); total testes, rag-i (O204); total liver, rag-i (0206); rat normal joint tissue (0300); and rat arthritic joint tissue (X300).

Samples for human mRNA isolation may include, e.g.: peripheral blood mononuclear cells (monocytes, T cells, NK cells, granulocytes, B cells), resting (Ti 00); peripheral blood mononuclear cells, activated with anti-CD3 for 2, 6, 12 h pooled (T101); T cell, THO clone Mot 72, resting (T1 02); T cell, THO clone Mot 72, activated with anti- CD28 and anti-CD3 for 3, 6, 12 h pooled (T1 03); T cell, THO clone Mot 72, anergic treated with specific peptide for 2, 7, 12 h pooled (T104); T cell, THi clone HY06, resting (T107); T cell, TH1 clone HY06, activated with anti-CD28 and anti-CD3 for 3, 6, 12 h pooled (T108); T cell, THi clone HY06, anergic treated with specific peptide for 2, 6, 12 h pooled (Ti09); T cell, TH2 clone HY935, resting (Ti 10); T cell, TH2 clone HY935, activated with anti-CD28 and anti-CD3 for 2, 7, 12 h pooled (TIl l ); T cells CD4+CD45RO- T cells polarized 27 days in anti-CD28, IL-4, and anti IFN-y, TH2 polarized, activated with anti-CD3 and anti-CD28 4 h (Ti 16); T cell tumor lines Jurkat and Hut78, resting (Ti 17); T cell clones, pooled AD130.2, Tc783.12, Tc783.13, Tc783.58, Tc782.69, resting (Ti 18); T cell random y6 T cell clones, resting (Ti 19); Splenocytes, resting (B 100); Splenocytes, activated with anti-CD40 and IL-4 (B 101); B cell EBV lines pooled WT49, RSB, JY, CVIR, 721.221, RM3, HSY, resting (B 102); B cell line JY, activated with PMA and ionomycin for 1, 6 h pooled (B103); NK 20 clones pooled, resting (Ki 00); NK 20 clones pooled, activated with PMA and ionomycin for 6 h (K101); NKL clone, derived from peripheral blood of LGL leukemia patient, IL-2 treated ((K106); NK cytotoxic clone 640-A30-1, resting (K107); hematopoietic precursor line TF1, activated with PMA and ionomycin for 1, 6 h pooled (C 100); U937 premonocytic line, resting (M100); U937 premonocytic line, activated with PMA and ionomycin for 1, 6 h pooled (MI 01); elutriated monocytes, activated with LPS, WNy, anti-IL-1O for 1, 2, 6, 12, 24 h pooled (M1 02); elutriated monocytes, activated with LPS, IFNy, IL-10 for 1, 2, 6, 12, 24 h pooled (M1 03); elutriated monocytes, activated with LPS, IFNy, anti-IL-10 for 4, 16 h pooled (M1 06); elutriated monocytes, activated with LPS, IFNy, IL-10 for 4, 16 h pooled (Ml 07); elutriated monocytes, activated LPS for 1 h (MI 08); elutriated monocytes, activated LPS for 6 h (M1 09); DC 70% CDla+, from CD34+GM-CSF, TNFoa 12 days, resting (DI01); DC 70% CDla+, from CD34+GM-CSF, TNFa 12 days, activated with PMA and ionomycin for 1 hr (D1102); DC 70% CD1a+, from CD34+GM- CSF, TNFa: 12 days, activated with PMA and ionomycin for 6 hr (D103); DC 95% CDla+, from CD34+GM-CSF, TNFa 12 days FACS sorted, activated with PMA and ionomycin for 1, 6 h pooled (D104); DC 95% CD 14+, ex CD34+GM-CSF, TNFa 12 days FACS sorted, activated with PMA and ionomycin 1, 6 hr pooled (D105); DC CDla+CD86+, from CD34+GM-CSF, TNFa 12 days FACS sorted, activated with PMA and ionomycin for 1, 6 h pooled (D 106); DC from monocytes GM-CSF, IL-4 5 days, resting (D107); DC from monocytes GM-CSF, IL-4 5 days, resting (D108); DC from monocytes GM-CSF, IL-4 5 days, activated LPS 4, 16 h pooled (DI 09); DC from monocytes GM- CSF, IL-4 5 days, activated TNFu, monocyte supe for 4, 16 h pooled (DI 10); leiomyoma LI i benign tumor (X101); normal myometrium M5 (0115); malignant leiomyosarcoma GSI (XI 03); lung fibroblast sarcoma line MRC5, activated with PMA and ionomycin for 1, 6 h pooled (CIOI); kidney epithelial carcinoma cell line CHA, activated with PMA and ionomycin for 1, 6 h pooled (C1 02); kidney fetal 28 wk male (O100); lung fetal 28 wk male (0101); liver fetal 28 wk male (0102); heart fetal 28 wk male (0103); brain fetal 28 wk male (0104); gallbladder fetal 28 wk male (0106); small intestine fetal 28 wk male (0107); adipose tissue fetal 28 wk male (0108); ovary fetal 25 wk female (O109); uterus fetal 25 wk female (O 110); testes fetal 28 wk male (Ol 11); spleen fetal 28 wk male (0112); adult placenta 28 wk (0113); and tonsil inflamed, from 12 year old (XI 00).

TaqMan quantitative PCR techniques have shown the DCRS6, in both mouse and human, to be expressed on T cells, including thymocytes and CD4+naive and differentiated (hDCRS6 is also expressed on dendritic cells), in gastrointestinal tissue, including stomach, intestine, colon and associated lymphoid tissue, e.g., Peyer's patches and mesenteric lymph nodes, and upregulated in inflammatory models of bowel disease, e.g., IL-10 KO mice. The hDCRS7 was detected in both resting and activated dendritic cells, epithelial cells, and mucosal tissues, including GI and reproductive tracts. These data suggest that family members are expressed in mucosal tissues and immune system cell types, and/or in gastrointestinal, airway, and reproductive tract development.

As such, therapeutic indications include, e.g., short bowel syndrome, post chemo/radio-therapy or alcoholic recovery, combinations with ulcer treatments or arthritis medication, Th2 pregnancy skewing, stomach lining/tissue regeneration, loss of adsorptive surface conditions, etc. See, e.g., Yamada, et al. (eds. 1999) Textbook of Gastroenterology; Yamada, et al. (eds. 1999) Textbook and Atlas of Gastroenterology; Gore and Levine (2000) Textbook of Gastrointestinal Radiology; and (1987) Textbook of Pediatric Gastroenterology.

Similar samples may isolated in other species for evaluation.

Primers specific for IL-1 7RA were designed and used in Taqman quantative PCR against various human libraries. IL- 17RA is highly expressed in innate immune myeloid cells including dendritic cells and monocytes. Expression is also detected in T-cell libraries. These data demonstrate the receptor is expressed in immune cell types and may be regulated by activation conditions.

| Table for IL-17RA | CT for IL- |
|---|---|
| library description | 17RA_H |
| DC ex monocytes GM-CSF, IL-4, resting | 16.97 |
| U937 premonocytic line, activated | 17.14 |
| DC ex monocytes GM-CSF, IL-4, resting | 17.53 |
| DC 70% CD1a +, ex CD34 + GM-CSF, TNFa, | 18.17 |
| resting | |
| monocytes, LPS, gIFN, anti-IL-10 | 18.27 |
| DC ex monocytes GM-CSF, IL-4, LPS | 18.51 |
| activated 4 + 16 hr | |
| DC ex monocytes GM-CSF, IL-4, monokine | 18.68 |
| activated 4 + 16 hr | |
| kidney epithelial carcinoma cell line CHA, | 18.69 |
| activated | |
| monocytes, LPS, 1 hr | 18.72 |
| monocytes, LPS, 6 hr | 18.72 |
| DC 70% CD1a +, ex CD34 + GM-CSF, TNFa, | 18.91 |
| activated 1 hr | |
| DC 70% CD1a +, ex CD34 + GM-CSF, TNFa, | 18.94 |
| activated 6 hr | |
| T cell, TH1 clone HY06, activated | 18.99 |
| lung fetal | 19.15 |
| T cell, TH1 clone HY06, resting | 19.18 |
| T cell, TH1 clone HY06, anergic | 19.23 |
| monocytes, LPS, gIFN, IL-10, 4 + 16 hr | 19.3 |
| spleen fetal | 19.51 |
| testes fetal | 19.7 |
| T cell, THO clone Mot 72, resting | 19.71 |
| T cell, THO clone Mot 72, resting | 19.84 |
| DC CD1a + CD86 +, ex CD34 + GM-CSF, TNFa, | 19.94 |
| activated 1 + 6 hr | |
| peripheral blood mononuclear cells, | 20.01 |
| activated | |
| hematopoietic precursor line TF1, activated | 20.07 |
| lung fibroblast sarcoma line MRC5, | 20.18 |
| activated | |
| Splenocytes, activated | 20.21 |
| T cell gd clones, resting | 20.27 |
| ovary fetal | 20.45 |
| T cells CD4 +, TH2 polarized, activated | 20.57 |
| Splenocytes, resting | 20.6 |
| uterus fetal | 20.62 |
| DC 95% CD1a +, ex CD34 + GM-CSF, TNFa, | 20.94 |
| activated 1 + 6 hr | |
| epithelial cells, unstimulated | 20.96 |
| peripheral blood mononuclear cells, resting | 20.97 |
| adipose tissue fetal | 21.13 |
| B cell line JY, activated | 21.28 |
| monocytes, LPS, gIFN, IL-10 | 21.37 |
| placenta 28 wk | 21.38 |
| NK 20 clones pooled, activated | 21.55 |
| pool of two normal human lung samples | 21.63 |
| normal human thyroid | 21.65 |
| epithelial cells, IL-1b activated | 21.72 |
| normal human skin | 21.84 |
| T cell, THO clone Mot 72, anergic | 21.87 |
| small intestine fetal | 22.01 |
| CD28- T cell clone in pME | 22.08 |
| T cell, TH2 clone HY935, activated | 22.09 |
| T cell clones, pooled, resting | 22.29 |
| Hashimoto's thyroiditis thyroid sample | 22.3 |
| NK 20 clones pooled, resting | 22.4 |
| B cell EBV lines, resting | 22.45 |
| T cell, TH2 clone HY935, resting | 22.86 |
| T cell, THO clone Mot 72, activated | 23.3 |
| monocytes, LPS, gIFN, anti-IL-10, 4 + 16 hr | 23.39 |
| T cell lines Jurkat and Hut78, resting | 23.4 |
| T cell, THO clone Mot 72, activated | 23.56 |
| Pneumocystic carnii pneumonia lung sample | 24.05 |
| 13937 premonocytic line, resting | 25.01 |
| pool of rheumatoid arthritis samples, human | 25.85 |
| pool of three heavy smoker human lung | 26.1 |
| samples | |
| DC 95% CD14 +, ex CD34 + GM-CSF, TNFa, | 32.69 |
| activated 1 + 6 hr | |
| kidney fetal | 33.7 |
| liver fetal | 34.4 |
| NK cytotoxic clone, resting | 34.49 |
| tonsil inflammed | 35.02 |
| normal w.t. monkey lung | 35.45 |
| gallbladder fetal | 35.84 |
| TR1 T cell clone | 35.86 |
| allergic lung sample | 36.39 |
| Psoriasis patient skin sample | 36.44 |
| normal human colon | 37.34 |
| brain fetal | 37.35 |
| Ascaris-challenged monkey lung, 4 hr. | 37.75 |
| Ascaris-challenged monkey lung, 24 hr. | 40 |
| heart fetal | 40 |
| normal w.t. monkey colon | 40 |
| ulcerative colitis human colon sample | 40 |

Primers specific for DCRS6_H were designed and used in Taqman quantative PCR against various human libraries. DCRS6_H is expressed in innate immune myeloid cells including dendritic cells and monocytes. Expression is also detected in T-cell libraries. These data demonstrate the receptor is expressed in immune cell types and may be regulated by activation conditions.

| Table for DCRS6_H | |
|---|---|
| library description | CT for DCRS6_H |
| T cell, THO clone Mot 72, resting | 15.54 |
| T cell, THO clone Mot 72, resting | 15.7 |
| DC ex monocytes GM-CSF, IL-4, resting | 17.84 |
| DC ex monocytes GM-CSF, IL-4, resting | 18.19 |
| DC ex monocytes GM-CSF, IL-4, LPS | 18.3 |
| activated 4 + 16 hr | |
| DC ex monocytes GM-CSF, IL-4, monokine | 18.3 |
| activated 4 + 16 hr | |
| T cell, TH1 clone HY06, resting | 18.43 |
| NK cytotoxic clone, resting | 18.53 |
| T cell clones, pooled, resting | 18.8 |
| T cell, TH1 clone HY06, activated | 19.03 |
| T cell, TH2 clone HY935, activated | 19.1 |
| TR1 T cell clone | 19.12 |
| T cells CD4 +, TH2 polarized, activated | 20.06 |
| B cell EBV lines, resting | 20.3 |
| T cell, TH2 clone HY935, resting | 20.48 |
| kidney epithelial carcinoma cell line CHA, | 21.07 |
| activated | |
| T cell, TH1 clone HY06, anergic | 21.14 |
| normal human colon | 21.29 |
| NK 20 clones pooled, resting | 21.49 |
| T cell gd clones, resting | 21.58 |
| gallbladder fetal | 22.21 |
| kidney fetal | 22.79 |
| liver fetal | 22.8 |
| Pneumocystic carnii pneumonia lung sample | 23.06 |
| CD28- T cell clone in pME | 23.18 |
| T cell, THO clone Mot 72, anergic | 23.2 |
| ovary fetal | 23.51 |
| normal human thyroid | 24.03 |
| small intestine fetal | 24.13 |
| testes fetal | 24.82 |
| epithelial cells, IL-1b activated | 26.08 |
| pool of three heavy smoker human lung | 26.49 |
| samples | |
| placenta 28 wk | 26.56 |
| normal w.t. monkey lung | 28.65 |
| peripheral blood mononuclear cells, | 33.39 |
| activated | |
| Ascaris-challenged monkey lung, 4 hr. | 36.59 |
| spleen fetal | 38.43 |
| peripheral blood mononuclear cells, resting | 40 |
| T cell, THO clone Mot 72, activated | 40 |
| T cell lines Jurkat and Hut78, resting | 40 |
| Splenocytes, resting | 40 |
| Splenocytes, activated | 40 |
| B cell line JY, activated | 40 |
| NK 20 clones pooled, activated | 40 |
| hematopoietic precursor line TF1, activated | 40 |
| U937 premonocytic line, resting | 40 |
| U937 premonocytic line, activated | 40 |
| monocytes, LPS, gIFN, anti-IL-10 | 40 |
| monocytes, LPS, gIFN, IL-10 | 40 |
| monocytes, LPS, gIFN, anti-IL-10, 4 + 16 hr | 40 |
| monocytes, LPS, gIFN, IL-10, 4 + 16 hr | 40 |
| monocytes, LPS, 1 hr | 40 |
| monocytes, LPS, 6 hr | 40 |
| DC 70% CD1 +, ex CD34 + GM-CSF, TNFa, | 40 |
| resting | |
| DC 70% CD1a +, ex CD34 + GM-CSF, TNFa, | 40 |
| activated 1 hr | |
| DC 70% CD1a +, ex CD34 + GM-CSF, TNFa, | 40 |
| activated 6 hr | |
| DC 95% CD1a +, ex CD34 + GM-CSF, TNFa, | 40 |
| activated 1 + 6 hr | |
| DC 95% CDb4 +, ex CD34 + GM-CSF, TNFa, | 40 |
| activated 1 + 6 hr | |
| DC CD1a + CD86 +, ex CD34 + GM-CSF, TNFa, | 40 |
| activated 1 + 6 hr | |
| epithelial cells, unstimulated | 40 |
| lung fibroblast sarcoma line MRC5, | 40 |
| activated | |
| Ascaris-challenged monkey lung, 24 hr. | 40 |
| pool of two normal human lung samples | 40 |
| allergic lung sample | 40 |
| normal w.t. monkey colon | 40 |
| ulcerative colitis human colon sample | 40 |
| Hashimoto's thyroiditis thyroid sample | 40 |
| pool of rheumatoid arthritis samples, human | 40 |
| normal human skin | 40 |
| Psoriasis patient skin sample | 40 |
| tonsil inflammed | 40 |
| lung fetal | 40 |
| heart fetal | 40 |
| brain fetal | 40 |
| adipose tissue fetal | 40 |
| uterus fetal | 40 |
| T cell, THO clone Mot 72, activated | 40 |

Primers specific for DCRS7_H were designed and used in Taqman quantative PCR against various human libraries. DCRS7_H is expressed in innate immune myeloid cells including dendritic cells and monocytes. Expression is also detected in fetal libraries. These data demonstrate the receptor is expressed in immune cell types and may be regulated by activation conditions.

| Table for DCRS7_H | CT for |
|---|---|
| library description | DCRS7_H |
| fetal uterus | 19.05 |
| DC mix | 19.34 |
| fetal small intestine | 19.46 |
| fetal ovary | 19.68 |
| fetal testes | 19.75 |
| fetal lung | 20.04 |
| CHA | 20.24 |
| normal thyroid | 20.32 |
| DC/GM/IL-4 | 20.52 |
| fetal spleen | 20.86 |
| normal lung | 20.94 |
| TF1 | 21 |
| allergic lung #19 | 21.02 |
| Psoriasis skin | 21.07 |
| fetal liver | 21.15 |
| MRC5 | 21.15 |
| 24 hr. Ascaris lung | 21.17 |
| hi dose IL-4 lung | 21.23 |
| CD1a + 95% | 21.32 |
| Hashimotos thyroiditis | 21.35 |
| Crohns colon 4003197A | 21.35 |
| normal lung pool | 21.36 |
| 70% DC resting | 21.42 |
| fetal kidney | 21.58 |
| adult placenta | 21.68 |
| lung 121897-1 | 21.8 |
| Pneumocystis carnii lung | 21.81 |
| #20 | |
| A549 unstim. | 21.89 |
| normal colon #22 | 21.94 |
| 18 hr. Ascaris lung | 22.09 |
| normal skin | 22.1 |
| Crohns colon 9609C144 | 22.13 |
| fetal adipose tissue | 22.35 |
| D6 | 22.39 |
| DC resting CD34-derived | 22.45 |
| DC TNF/TGFb act CD34-der. | 22.54 |
| fetal brain | 22.9 |
| DC CD40L activ. mono- | 22.91 |
| deriv. | |
| Crohns colon 403242A | 22.91 |
| ulcerative colitis colon | 23 |
| #26 | |
| RA synovium pool | 23.06 |
| A549 activated | 23.06 |
| mono + IL-10 | 23.42 |
| DC LPS | 23.49 |
| Mot 72 activated | 23.66 |
| CD1a + CD86 + | 23.86 |
| HY06 resting | 23.87 |
| U937 activated | 23.97 |
| inflammed tonsil | 23.97 |
| D1 | 24.06 |
| M1 | 24.17 |
| CD14 + 95% | 24.21 |
| lung 080698-2 | 24.28 |
| 4 hr. Ascaris lung | 24.37 |
| Jurkat activated pSPORT | 24.42 |
| DC resting mono-derived | 24.48 |
| HY06 activated | 24.54 |
| C+ | 24.64 |
| Splenocytes resting | 24.65 |
| U937/CD004 resting | 24.96 |
| PBMC resting | 25.8 |
| Mot 72 resting | 25.91 |
| mono + anti-IL-10 | 26.14 |
| NK pool | 26.99 |
| HY06 anti-peptide | 27.34 |
| mast cell pME | 27.38 |
| Tc gamma delta | 28.14 |
| TC1080 CD28- pMET7 | 31.05 |
| PBMC activated | 31.89 |
| NK non cytotox. | 32.3 |
| RV-C30 TR1 pMET7 | 32.5 |
| Bc | 33.72 |
| C− | 33.8 |
| Splenocytes activated | 34.7 |
| JY | 35.05 |
| NK cytotox. | 36.44 |
| NKL/IL-2 | 37.59 |
| HY935 resting | 37.6 |
| NK pool activated | 38.15 |
| Mot 72 anti-peptide | 38.87 |
| fetal heart | 40.92 |
| B21 resting | 42.05 |
| Jurkat resting pSPORT | 42.8 |
| B21 activated | 43.09 |
| NKAG pSPORT | 44.85 |
| HY935 activated | 45 |
| M6 | 45 |

Primers specific for DCRS9_H were designed and used in Taqman quantative PCR against various human libraries. DCRS9_H is expressed T-cells, fetal lung, and resting monocytes. These data demonstrate the receptor is expressed in immune cell types and may be regulated by activation conditions.

| Table for DCRS9_H | CT for |
|---|---|
| library description | DCRS9_H |
| HY06 resting | 22.35 |
| fetal lung | 22.63 |
| HY06 anti-peptide | 22.72 |
| HY06 activated | 22.96 |
| U937/CD004 resting | 24.16 |
| fetal small | 24.94 |
| intestine | |
| JY | 25.04 |
| Mot 72 resting | 25.12 |
| Jurkat activated | 25.2 |
| pSPORT | |
| RV-C30 TR1 pMET7 | 26.51 |
| fetal kidney | 26.76 |
| MRC5 | 27.2 |
| Psoriasis skin | 27.3 |
| Tc gamma delta | 27.37 |
| Crohns colon | 27.44 |
| 4003197A | |
| fetal spleen | 27.72 |
| normal lung | 27.83 |
| Hashimotos | 28.03 |
| thyroiditis | |
| B21 resting | 28.32 |
| TF1 | 28.39 |
| NK cytotox. | 28.44 |
| TC1080 CD28- pMET7 | 28.61 |
| Pneumocystis carnii | 29.05 |
| lung #20 | |
| U937 activated | 29.06 |
| HY935 resting | 29.09 |
| CD1a + 95% | 29.13 |
| B21 activated | 29.2 |
| Mot 72 activated | 29.21 |
| fetal testes | 29.27 |
| lung 080698-2 | 29.32 |
| Jurkat resting | 29.38 |
| pSPORT | |
| CD14 + 95% | 29.38 |
| normal thyroid | 29.53 |
| Mot 72 anti- | 29.65 |
| peptide | |
| Splenocytes | 29.85 |
| resting | |
| Crohns colon | 30.28 |
| 9609C144 | |
| lung 121897-1 | 30.37 |
| 24 hr. Ascaris lung | 30.59 |
| hi dose IL-4 lung | 30.8 |
| CD1a + CD86 + | 31.42 |
| normal skin | 31.73 |
| fetal uterus | 31.79 |
| PBMC activated | 31.82 |
| inflammed tonsil | 31.98 |
| fetal brain | 32.21 |
| PA synovium pool | 32.77 |
| allergic lung #19 | 33.18 |
| 18 hr. Ascaris lung | 33.42 |
| adult placenta | 33.43 |
| normal lung pool | 33.45 |
| Crohns colon | 33.52 |
| 403242A | |
| NK pool | 33.72 |
| HY935 activated | 33.75 |
| DC/GM/IL-4 | 34.28 |
| DC resting mono- | 34.57 |
| derived | |
| fetal ovary | 35.06 |
| fetal adipose | 35.07 |
| tissue | |
| CHA | 35.2 |
| PBMC resting | 35.95 |
| Bc | 36.19 |
| A549 unstim. | 36.4 |
| fetal heart | 36.87 |
| ulcerative colitis | 37.83 |
| colon #26 | |
| C− | 38.32 |
| 4 hr. Ascaris lung | 40.2 |
| D6 | 40.62 |
| C+ | 44.38 |
| A549 activated | 44.58 |
| Splenocytes | 45 |
| activated | |
| NK pool activated | 45 |
| NKAG pSPORT | 45 |
| NKL/IL-2 | 45 |
| NK non cytotox. | 45 |
| mono + anti-IL-10 | 45 |
| mono + IL-10 | 45 |
| M1 | 45 |
| M6 | 45 |
| 70% DC resting | 45 |
| D1 | 45 |
| DC LPS | 45 |
| DC mix | 45 |
| fetal liver | 45 |
| mast cell pME | 45 |
| DC CD40L activ. | 45 |
| mono-deriv. | |
| DC resting CD34- | 45 |
| derived | |
| DC TNF/TGFb act | 45 |
| CD34-der. | |
| normal colon #22 | 45 |

### V. Cloning of species counterparts

Various strategies are used to obtain species counterparts of the DCRSs, preferably from other primates or rodents. One method is by cross hybridization using closely related species DNA probes. It may be useful to go into evolutionarily similar species as intermediate steps. Another method is by using specific PCR primers based on the identification of blocks of similarity or difference between genes, e.g., areas of highly conserved or nonconserved polypeptide or nucleotide sequence. Sequence database searches may identify species counterparts. VI. Production of mammalian protein An appropriate, e.g., GST, fusion construct is engineered for expression, e.g., in E. coli. For example, a mouse IGIF pGex plasmid is constructed and transformed into E. coli. Freshly transformed cells are grown, e.g., in LB medium containing 50 ttg/ml ampicillin and induced with IPTG (Sigma, St. Louis, MO). After overnight induction, the bacteria are harvested and the pellets containing the appropriate protein are isolated. The pellets are homogenized, e.g., in TE buffer (50 mM Tris-base pH 8.0, 10 mM EDTA and 2 mM pefabloc) in 2 liters. This material is passed through a microfluidizer (Microfluidics, Newton, MA) three times. The fluidized supernatant is spun down on a Sorvall GS-3 rotor for 1 h at 13,000 rpm. The resulting supernatant containing the cytokine receptor protein is filtered and passed over a glutathione-SEPHAROSE column equilibrated in 50 mM Tris-base pH 8.0. Fractions containing the DCRS8-GST fusion protein are pooled and cleaved, e.g., with thrombin (Enzyme Research Laboratories, Inc., South Bend, IN). The cleaved pool is then passed over a Q-SEPHAROSE column equilibrated in 50 mM Tris-base. Fractions containing DCRS8 are pooled and diluted in cold distilled H20, to lower the conductivity, and passed back over a fresh Q-Sepharose column, alone or in succession with an immunoaffinity antibody column. Fractions containing the DCRS8 protein are pooled, aliquoted, and stored in the -70° C. freezer.

Comparison of the CD spectrum with cytokine receptor protein may suggest that the protein is correctly folded. See Hazuda, et al. (1969) J. Biol. Chem. 264:1689-1693.

### VII. Preparation of specific antibodies

Inbred Balb/c mice are immunized intraperitoneally with recombinant forms of the protein, e.g., purified DCRS8 or stable transfected NIH-3T3 cells. Animals are boosted at appropriate time points with protein, with or without additional adjuvant, to further stimulate antibody production. Serum is collected, or hybridomas produced with harvested spleens.

Alternatively, Balb/c mice are immunized with cells transformed with the gene or fragments thereof, either endogenous or exogenous cells, or with isolated membranes enriched for expression of the antigen. Serum is collected at the appropriate time, typically after numerous further administrations. Various gene therapy techniques may be useful, e.g., in producing protein in situ, for generating an immune response. Serum or antibody preparations may be cross-absorbed or immunoselected to prepare substantially purified antibodies of defined specificity and high affinity.

Monoclonal antibodies may be made. For example, splenocytes are fused with an appropriate fusion partner and hybridomas are selected in growth medium by standard procedures. Hybridoma supernatants are screened for the presence of antibodies which bind to the DCRS8, e.g., by ELISA or other assay. Antibodies which specifically recognize specific DCRS8 embodiments may also be selected or prepared.

In another method, synthetic peptides or purified protein are presented to an immune system to generate monoclonal or polyclonal antibodies. See, e.g., Coligan (ed. 1991) Current Protocols in Immunology Wiley/Greene; and Harlow and Lane (1989) Antibodies: A Laboratory Manual Cold Spring Harbor Press. In appropriate situations, the binding reagent is either labeled as described above, e.g., fluorescence or otherwise, or immobilized to a substrate for panning methods. Nucleic acids may also be introduced into cells in an animal to produce the antigen, which serves to elicit an immune response. See, e.g., Wang, et al. (1993) Proc. Nat'l. Acad. Sci. 90:4156-4160; Barry, et al. (1994) BioTechniques 16:616-619; and Xiang, et al. (1995) Immunity 2: 129-135.

### VIII. Production of fusion proteins

Various fusion constructs are made with DCRS8 or DCRS9. A portion of the appropriate gene is fused to an epitope tag, e.g., a FLAG tag, or to a two hybrid system construct. See, e.g., Fields and Song (1989) Nature 340:245-246.

The epitope tag may be used in an expression cloning procedure with detection with anti-FLAG antibodies to detect a binding partner, e.g., ligand for the respective cytokine receptor. The two hybrid system may also be used to isolate proteins which specifically bind to the receptor subunit.

### IX. Structure activity relationship

Information on the criticality of particular residues is determined using standard procedures and analysis. Standard mutagenesis analysis is performed, e.g., by generating many different variants at determined positions, e.g., at the positions identified above, and evaluating biological activities of the variants. This may be performed to the extent of determining positions which modify activity, or to focus on specific positions to determine the residues which can be substituted to either retain, block, or modulate biological activity.

Alternatively, analysis of natural variants can indicate what positions tolerate natural mutations. This may result from populational analysis of variation among individuals, or across strains or species. Samples from selected individuals are analyzed, e.g., by PCR analysis and sequencing. This allows evaluation of population polymorphisms.

### X. Isolation of a ligand

A cytokine receptor can be used as a specific binding reagent to identify its binding partner, by taking advantage of its specificity of binding, much like an antibody would be used. The binding receptor may be a heterodimer of receptor subunits; or may involve, e.g., a complex of the DCRS8 with another cytokine receptor subunit. A binding reagent is either labeled as described above, e.g., fluorescence or otherwise, or immobilized to a substrate for panning methods.

The binding composition is used to screen an expression library made from a cell line which expresses a binding partner, i.e., ligand, preferably membrane associated. Standard staining techniques are used to detect or sort surface expressed ligand, or surface expressing transformed cells are screened by panning. Screening of intracellular expression is performed by various staining or immunofluorescence procedures. See also McMahan, et al. (1991) EMBO J. 10:2821-2832.

For example, on day 0, precoat 2-chamber permanox slides with 1 ml per chamber of fibronectin, 10 ng/ml in PBS, for 30 min at room temperature. Rinse once with PBS. Then plate COS cells at 2-3 x 105 cells per chamber in 1.5 ml of growth media. Incubate overnight at 37 C.

On day 1 for each sample, prepare 0.5 ml of a solution of 66 [tg/ml DEAE-dextran, 66 tM chloroquine, and 4 pg DNA in serum free DME. For each set, a positive control is prepared, e.g., of DCRS8-FLAG cDNA at 1 and 1/200 dilution, and a negative mock. Rinse cells with serum free DME. Add the DNA solution and incubate 5 hr at 37 C. Remove the medium and add 0.5 ml 10% DMSO in DME for 2.5 min. Remove and wash once with DME. Add 1.5 ml growth medium and incubate overnight.

On day 2, change the medium. On days 3 or 4, the cells are fixed and stained. Rinse the cells twice with Hank's Buffered Saline Solution (HBSS) and fix in 4% paraformaldehyde (PFA)/glucose for 5 min. Wash 3X with HBSS. The slides may be stored at -80 C after all liquid is removed. For each chamber, 0.5 ml incubations are performed as follows. Add HBSS/saponin (0.1%) with 32 1μl/ml of 1 M NaN₃ for 20 min. Cells are then washed with HBSS/saponin 1X. Add appropriate DCRS8 or DCRS8/antibody complex to cells and incubate for 30 min. Wash cells twice with HBSS/saponin. If appropriate, add first antibody for 30 min. Add second antibody, e.g., Vector anti-mouse antibody, at 1/200 dilution, and incubate for 30 min. Prepare ELISA solution, e.g., Vector Elite ABC horseradish peroxidase solution, and preincubate for 30 min. Use, e.g., 1 drop of solution A (avidin) and 1 drop solution B (biotin) per 2.5 ml HBSS/saponin. Wash cells twice with HBSS/saponin. Add ABC HRP solution and incubate for 30 min. Wash cells twice with HBSS, second wash for 2 min, which closes cells. Then add Vector diaminobenzoic acid (DAB) for 5 to 10 min. Use 2 drops of buffer plus 4 drops DAB plus 2 drops of H₂0₂ per 5 ml of glass distilled water. Carefully remove chamber and rinse slide in water. Air dry for a few minutes, then add 1 drop of Crystal Mount and a cover slip. Bake for 5 min at 85-90 C.

Evaluate positive staining of pools and progressively subelone to isolation of single genes responsible for the binding.

Alternatively, receptor reagents are used to affinity purify or sort out cells expressing a putative ligand. See, e.g., Sambrook, et al. or Ausubel, et al.

Another strategy is to screen for a membrane bound receptor by panning. The receptor cDNA is constructed as described above. Immobilization may be achieved by use of appropriate antibodies which recognize, e.g., a FLAG sequence of a DCRS8 fusion construct, or by use of antibodies raised against the first antibodies. Recursive cycles of selection and amplification lead to enrichment of appropriate clones and eventual isolation of receptor expressing clones.

Phage expression libraries can be screened by mammalian DCRS8. Appropriate label techniques, e.g., anti-FLAG antibodies, will allow specific labeling of appropriate clones.

We tested the ability of DCRS receptors to specifically bind IL-17 family cytokines. Recombinant FLAG-hIL- 17 family cytokines were used in binding experiments on BaU3 DCRS receptor transfected expressing recombinant IL-17R_H, DCRS6_H, DCRS7_H, DCRS8_H and DCRS9_H and analyzed by FACS. We can demonstrate specific binding of IL-17 family member IL-74 to DCRS6 expressing Baf/3 cells. In additional experiments we have shown IL-17 specific binding to IL-17R_H, DCRS7_H, DCRS8_H. Further experiments show IL-71 binding to DCRS8_Hu transfectants. These experiments demonstrate the sequence homology among IL-17 related cytokine receptors confers finctional binding to IL-1 7 cytokines.

All citations herein are incorporated herein by reference to the same extent as if each individual publication or patent application was specifically and individually indicated to be incorporated by reference.

Many modifications and variations of this invention can be made without departing from its spirit and scope, as will be apparent to those skilled in the art. The specific embodiments described herein are offered by way of example only, and the invention is to be limited by the terms of the appended claims, along with the full scope of equivalents to which such claims are entitled; and the invention is not to be limited by the specific embodiments that have been presented herein by way of example.

What is claimed is:

1. A composition of matter selected from:
a) a substantially pure or recombinant polypeptide comprising at least three distinct nonoverlapping segments of at least four amino acids identical to segments of SEQ ID NO: 14;
b) a substantially pure or recombinant polypeptide comprising at least two distinct nonoverlapping segments of at least five amino acids identical to segments of SEQ ID NO: 14;
c) a natural sequence DCRS8 comprising mature SEQ ID NO: 14;
d) a fusion polypeptide comprising DCRS8 sequence;
e) a substantially pure or recombinant polypeptide comprising at least three distinct nonoverlapping segments of at least four amino acids identical to segments of SEQ ID NO: 17 or 20;
f) a substantially pure or recombinant polypeptide comprising at least two distinct nonoverlapping segments of at least five amino acids identical to segments of SEQ ID NO: 17 or 20;
g) a natural sequence DCRS9 comprising mature SEQ ID NO: 17 or 20; or
h) a fusion polypeptide comprising DCRS9 sequence.

2. The substantially pure or isolated antigenic polypeptide of claim 1, wherein said distinct nonoverlapping segments of identity include:
a) one of at least eight amino acids;
b) one of at least four amino acids and a second of at least five amino acids;
c) at least three segments of at least four, five, and six amino acids, or
d) one of at least twelve amino acids.

3. The composition of matter of claim 1, wherein said:
a) polypeptide:
i) comprises a mature sequence of Table 3 or 4;
ii) is an unglycosylated form of DCRS8 or DCRS9;
iii) is from a primate, such as a human;
iv) comprises at least seventeen amino acids of SEQ ID NO: 14 or 17;
v) exhibits at least four nonoverlapping segments of at least seven amino acids of SEQ ID NO: 14 or 17;
vi) is a natural allelic variant of DCRS8 or DCRS9;
vii) has a length at least about 30 amino acids;
viii) exhibits at least two non-overlapping epitopes which are specific for a primate DCRS8 or DCRS9;
ix) is glycosylated;
x) has a molecular weight of at least 30 kD with natural glycosylation;
xi) is a synthetic polypeptide;
xii) is attached to a solid substrate;
xiii) is conjugated to another chemical moiety;
xiv) is a 5-fold or less substitution from natural sequence; or
xv) is a deletion or insertion variant from a natural sequence.

4. A composition comprising:
a) a substantially pure DCRS8 or DCRS9 and another cytokine receptor family member;
′ z′ 2′ -m′ b) a sterile DCRS8 or DCRS9 polypeptide of claim 1;
c) said DCRS8 or DCRS9 polypeptide of claim 1 and a carrier, wherein said carrier is:
i) an aqueous compound, including water, saline, and/or buffer; and/or
ii) formulated for oral, rectal, nasal, topical, or parenteral administration.

5. The fusion polypeptide of claim 1, comprising:
a) mature protein sequence of Table 3 or 4;
b) a detection or purification tag, including a FLAG, His6, or 1 g sequence; or
c) sequence of another cytokine receptor protein.

6. A kit comprising a polypeptide of claim 1, and:
a) a compartment comprising said protein or polypeptide; or
b) instructions for use or disposal of reagents in said kit.

7. A binding compound comprising an antigen binding site from an antibody, which specifically binds to a natural DCRS8 or DCRS9 polypeptide of claim 1, wherein:
a) said binding compound is in a container;
b) said DCRS8 or DCRS9 polypeptide is from a human;
c) said binding compound is an Fv, Fab, or Fab2 fragment;
d) said binding compound is conjugated to another chemical moiety; or
e) said antibody:
i) is raised against a peptide sequence of a mature polypeptide of Table 3 or4;
ii) is raised against a mature DCRS8 or DCRS9;
iii) is raised to a purified human DCRS8 or DCRS9;
iv) is immunoselected;
v) is a polyclonal antibody;
vi) binds to a denatured DCRS8 or DCRS9;
vii) exhibits a Kd to antigen of at least 30 HM;
viii) is attached to a solid substrate, including a bead or plastic membrane;
ix) is in a sterile composition; or
x) is detectably labeled, including a radioactive or fluorescent label.

8. A kit comprising said binding compound of claim 7, and:
a) a compartment comprising said binding compound; or
b) instructions for use or disposal of reagents in said kit.

9. A method of producing an antigen:antibody complex, comprising contacting under appropriate conditions a primate DCRS8 or DCRS9 polypeptide with an antibody of claim 7, thereby allowing said complex to form.

10. The method of claim 9, wherein:
a) said complex is purified from other cytokine receptors;
b) said complex is purified from other antibody;
c) said contacting is with a sample comprising an interferon;
d) said contacting allows quantitative detection of said antigen;
e) said contacting is with a sample comprising said antibody; or
f) said contacting allows quantitative detection of said antibody.

11. A composition comprising:
a) a sterile binding compound of claim 7, or
b) said binding compound of claim 7 and a carrier, wherein said carrier is:
i) an aqueous compound, including water, saline, and/or buffer; and/or
ii) formulated for oral, rectal, nasal, topical, or parenteral administration.

12. An isolated or recombinant nucleic acid encoding said polypeptide of claim 1, wherein said:
a) DCRS8 or DCRS9 is from a human; or
b) said nucleic acid:
i) encodes an antigenic peptide sequence of Table 3 or 4;
ii) encodes a plurality of antigenic peptide sequences of Table 3 or 4;
iii) exhibits identity over at least thirteen nucleotides to a natural cDNA encoding said segment;
iv) is an expression vector;
v) further comprises an origin of replication;
vi) is from a natural source;
vii) comprises a detectable label;
viii) comprises synthetic nucleotide sequence;
ix) is less than 6 kb, preferably less than 3 kb;
x) is from a primate;
xi) comprises a natural full length coding sequence;
xii) is a hybridization probe for a gene encoding said DCRS8 or DCRS9;
or
xiii) is a PCR primer, PCR product, or mutagenesis primer.

13. A cell or tissue comprising said recombinant nucleic acid of claim 12.

14. The cell of claim 13, wherein said cell is:
a) a prokaryotic cell;
b) a eukaryotic cell;
c) a bacterial cell;
d) a yeast cell;
e) an insect cell;
f) a mammalian cell;
g) a mouse cell;
h) a primate cell; or
i) ahumancell.

15. A kit comprising said nucleic acid of claim 12, and:
a) a compartment comprising said nucleic acid;
b) a compartment further comprising a primate DCRS8 or DCRS9 polypeptide;
or
c) instructions for use or disposal of reagents in said kit.

16. A nucleic acid which:
a) hybridizes under wash conditions of 30 minutes at 30° C and less than 2M salt to the coding portion of SEQ ID NO: 13 or 16; or
b) exhibits identity over a stretch of at least about 30 nucleotides to a primate DCRS8 or DCRS9.

17. The nucleic acid of claim 16, wherein:
a) said wash conditions are at 450 C and/or 500 mM salt; or
b) said stretch is at least 55 nucleotides.

18. The nucleic acid of claim 16, wherein:
a) said wash conditions are at 55° C. and/or 150 mM salt; or
b) said stretch is at least 75 nucleotides.

19. A method of modulating physiology or development of a cell or tissue culture cells comprising contacting said cell with an agonist or antagonist of a mammalian DCRS8 or DCRS9.

20. The method of claim 19, wherein said cell is transformed with a nucleic acid encoding said DCRS8 or DCRS9 and another cytokine receptor subunit.
